(19) United States
(12) Patent Application Publication
(10) Pub. No.: US 2017/0312148 A1
(43) Pub. Date: Nov. 2, 2017

(54) FLUID-ABSORBENT ARTICLE

(71) Applicant: BASF SE, Ludwigshafen (DE)

(72) Inventors: Katarzyna Dobrosielska-Oura, Ludwigshafen (DE); Christophe Bauduin, Plankstadt (DE); Markus Tönnessen, Ludwigshafen (DE); Mark Elliott, Gruenstadt (DE); Markus Vogt, Ludwigshafen (DE); Thomas Daniel, Waldsee (DE); Norbert Herfert, Altenstadt (DE); Marc Haefner, Worms (DE); Alexandra Faubel, Laudenbach (DE)

(73) Assignee: BASF SE, Ludwigshafen Am Rhein (DE)

(21) Appl. No.: 15/520,092

(22) PCT Filed: Oct. 14, 2015
(86) PCT No.: PCT/EP2015/073754

§ 371 Date: Apr. 19, 2017

(30) Foreign Application Priority Data
Oct. 24, 2014 (EP) 14190292.4
Feb. 5, 2015 (EP) 15153956.6

(51) Int. Cl. A61F 13/537; A61L 15/60; A61L 15/22; A61F 13/539; A61F 13/15; A61F 13/534; A61F 13/53

(52) U.S. Cl. CPC A61F 13/53717; A61F 13/539; A61L 15/60; A61L 15/225; A61F 13/15203; A61F 2013/15406; A61F 2013/530591; A61F 2013/5395; A61F 2013/5349

(57) ABSTRACT

The present invention relates to a fluid-absorbent article, comprising a fluid-absorbent core, comprising from 0 to 90% by weight fibrous material and from 10 to 100% by weight water-absorbent polymer particles having a mean sphericity (SPHT) from 0.8 to 0.95, based on the sum of water-absorbent polymer particles and fibrous material;

and an acquisition-distribution layer between having a three dimensional apertured structure having a femal side (5) and a male side (6), wherein the femal side presents a multiplicity of openings extending in the form of through holes in the direction of a lower surface of the same structure being the male side, wherein the quantity of the water absorbent polymer particles within the fluid absorbent core is at least 3 g.

The present invention relates to a fluid-absorbent article, comprising a fluid-absorbent core, comprising from 0 to 90% by weight fibrous material and from 10 to 100% by weight water-absorbent polymer particles having a mean sphericity (SPHT) from 0.8 to 0.95, based on the sum of water-absorbent polymer particles and fibrous material;

and an acquisition-distribution layer between having a three dimensional apertured structure having a femal side (5) and a male side (6), wherein the femal side presents a multiplicity of openings extending in the form of through holes in the direction of a lower surface of the same structure being the male side, wherein the quantity of the water absorbent polymer particles within the fluid absorbent core is at least 3 g.

The preparation of water-absorbing polymer particles is likewise described in the monograph “Modern Superabsorbent Polymer Technology”, F. L. Buchholz and A. T. Graham, Wiley-VCH, 1998, pages 71 to 103. The water-absorbing polymer particles are also referred to as “fluid-absorbing polymer particles”, “superabsorbent polymers” or “superabsorbents”.

The preparation of water-absorbent polymer particles by polymerizing droplets of a monomer solution is described, for example, in EP 0 348 180 A1, WO 96/40427 A1, U.S. Pat. No. 5,269,980, WO 2008/009580 A1, WO 2008/052971 A1, WO2011/026876 A1, WO 2011/117263 A1 and WO 2014/079694.

Polymerization of monomer solution droplets in a gas phase surrounding the droplets (“dropletization polymerization”) affords round water-absorbent polymer particles of high mean sphericity (mSPHT). The mean sphericity is a measure of the roundness of the polymer particles and can be determined, for example, with the Camsizer® image analysis system (Retsch Technology GmbH; Haan; Germany).

The production of fluid-absorbent articles is described in the monograph “Modern Superabsorbent Polymer Technology”, F. L. Buchholz and A. T. Graham, Wiley-VCH, 1998, pages 252 to 258.

Fluid-absorbent articles consist typically of an upper liquid-pervious top-sheet, a lower liquid-impervious layer, an acquisition-distribution layer and fluid-absorbing composite between the top-sheet and the liquid-impervious layer. The composite consists of water-absorbing polymers and fibers. Further layers are, for example tissue layers.

Usually the several layers of fluid-absorbent articles fulfill definite functions such as dryness for the upper liquid-pervious layer, vapor permeability without wetting through for the lower liquid-impervious layer, a flexible, vapor permeable and thin fluid-absorbent core, showing fast absorption rates and being able to retain highest quantities of body fluids, and an acquisition-distribution layer between the upper layer and the core, acting as transport and distribution layer of the discharged body fluids.

An acquisition-distribution layer, referred to in short as ADL, is arranged underneath the top sheet. The acquisition-distribution layer usually comprises masses of fibers, i.e. chemically stiffened, twisted, curled cellulosic fibers, non-woven fibrous webs or three dimensional formed webs with apertures or the acquisition distribution layer may also be made of a three dimensional formed film with apertures (e. g. U.S. Pat. No. 7,378,568).

The layer should ensure that the body liquids pass rapidly inside the structure of the absorbent article and are distributed uniformly throughout the thickness of the underlying storage layer or core instead of being absorbed in a localized manner only in the zones located underneath the points where the liquid arrives, or mainly in these zones. But often a distribution throughout the total core is not ensured.

Furthermore the fluid-acquisition time of these usual non-woven acquisition-distribution layers is relatively long with a high risk of leakage especially in gush situations.

To prevent leakage and wet feeling it is preferred to have thicker acquisition-distribution layer's so that the time to absorb the body fluid is preferably short, in case of usual non-woven acquisition-distribution layers formed by a random or preferably oriented distribution of fibres, the standard weight in diapers are 30 to 180 g/m². But this contravenes the trend to thinner absorbent articles, as the thickness is also a great issue in respect to absorbent articles especially in respect to noticeability for adult articles and also hindrance, especially for baby diapers and pants.

It is therefore an object of the present invention to provide ultrathin fluid-absorbent articles with improved liquid acquisition and retention behavior.

It is also an object of the present invention to provide ultrathin fluid-absorbent articles which ensure a fluid distribution throughout the total absorbent core.

It is furthermore an object of the present invention to provide ultrathin fluid-absorbent articles with improved rewet performance.

The object is achieved by a fluid-absorbent article, comprising
(A) an upper liquid-pervious layer,
(B) a lower liquid-impervious layer,
(C) a fluid-absorbent core between the layer (A) and the layer (B), comprising from 0 to 90% by weight fibrous material and from 10 to 100% by weight water-absorbent polymer particles having a mean sphericity (SPHT) from 0.8 to 0.95, based on the sum of water-absorbent polymer particles and fibrous material;
(D) an acquisition-distribution layer between (A) and (C) having a three dimensional apertured structure having a femal side (5) and a male side (6), wherein the femal side presents a multiplicity of openings extending in the form of through holes in the direction of a lower surface of the same structure being the male side,
(E) an optional tissue layer disposed immediately above and/or below (C); and
(F) other optional components,
wherein the quantity of the water absorbent polymer particles within the fluid absorbent core is at least 3 g.

The object is also achieved by a fluid-absorbent article, comprising
(A) an upper liquid-pervious layer,
(B) a lower liquid-impervious layer,
(C) a fluid-absorbent core between the layer (A) and the layer (B), comprising from 0 to 50% by weight fibrous material and from 50 to 100% by weight water-absorbent polymer particles having a mean sphericity (SPHT) from 0.8 to 0.95, based on the sum of water-absorbent polymer particles and fibrous material;
(D) an acquisition-distribution layer between (A) and (C) having a three dimensional apertured structure having a femal side (5) and a male side (6), wherein the femal side presents a multiplicity of openings extending in the form of through holes in the direction of a lower surface of the same structure being the male side,
(E) an optional tissue layer disposed immediately above and/or below (C); and
(F) other optional components,
wherein the quantity of the water absorbent polymer particles within the fluid absorbent core is at least 3 g.

Preferably the acquisition-distribution layer (D) provides mutually adjacent through holes of the acquisition-distribution layer (D) being separated by segments (10) having a profile with symmetrical sides converging towards the female side.

Wherein according to one embodiment of the present invention the base (8) with a length D1 of the segment is at the male side and the apex (9) is at the female side.

According to the invention the through holes have an area at the female side (D2) which is greater than the area of the through holes at the male side (D3), wherein preferably the sum of the areas D2 of the acquisition-distribution layer (D) is at least 50% of the total area of the acquisition-distribution layer (D)

According to another embodiment of the invention the acquisition-distribution layer (D) may be placed so that the female side faces the upper layer (A) and the male side faces the absorbent core (C).

But it is also preferred that the acquisition-distribution layer (D) may be placed so that male side of the acquisition-distribution layer (D) faces the upper layer (A) and the female side faces the absorbent core (C).

According to one embodiment of the present invention the acquisition-distribution layer is a three dimensional formed film. Preferably the acquisition-distribution layer is a perforated plastic film as e.g. disclosed in U.S. Pat. No. 7,378,568 or WO 00/62729.

According to another embodiment of the present invention the acquisition-distribution layer is a three dimensional apertured web, wherein the apertures are defined by one or more sidewalls in the web which extend from a first side of the web and protrude from a second side of the web.

The openings or apertures in the web or film preferably imparted with a pattern. The pattern may be hexagonal, circular, oval, elliptical, polygonal or any other suitable patterns or combinations thereof.

Apertured three dimensional films usually having a basis weight between 20 and 30 g/m².

The water-absorbing particles used in the inventive fluid-absorbent article provide a mean sphericity of at least 0.8. The particles itself feel soft and the coarse feeling even in high loaded water-absorbent articles is reduced. The fluid-absorbent articles therefore provide also improved haptic properties.

It is preferred that the amount of the basis weight of the acquisition-distribution layer (D) in gsm is less than the amount of water-absorbent polymer particles contained in the fluid absorbent core (C) in % by weight, based on the sum of water-absorbent polymer particles and fibrous material.

It is further preferred that the water-absorbent polymer particles have a centrifuge retention capacity of at least 10 g/g and an absorbency under high load of at least 7 g/g.

The fluid-absorbent polymer particles according to one embodiment of the present invention have a centrifuge retention capacity (CRC) of typically at least 10 g/g,

preferably at least 15 g/g, preferentially at least 20 g/g, more preferably at least 25 g/g,

The water-absorbent polymer particles therefore possess a high centrifuge retention capacity which impart good liquid distribution when used in hygiene articles.

Furthermore fluid-absorbent articles according to one embodiment of the present invention maintaining excellent dryness, independent of the absolute amounts of water-absorbent particles. Even small amounts such as e.g. 10 g, 9 g, 8 g or 6 g for a maxi diaper (size L), or 5 g for a midi diaper or less are sufficient.

The fluid-absorbent articles according to one embodiment of the present invention, comprising water-absorbent polymer particles and less than 15% by weight fibrous material and/or adhesives in the absorbent core.

Suitable water-absorbent polymers are produced by a process, comprising the steps forming water-absorbent polymer particles by polymerizing a monomer solution, wherein the content of residual monomers in the water-absorbent polymer particles is in the range from 0.03 to 15% by weight.

Suitable water-absorbent polymers are produced by a process, comprising the steps forming water-absorbent polymer particles by polymerizing a monomer solution, coating of water-absorbent polymer particles with at least one surface-postcrosslinker and thermal surface-postcrosslinking of the coated water-absorbent polymer particles, wherein the content of residual monomers in the water-absorbent polymer particles prior to the coating with the surface-postcrosslinker is in the range from 0.03 to 15% by weight, and the temperature during the thermal surface-postcrosslinking is in the range from 100 to 180° C.

Suitable water-absorbent polymers can be also produced by a process, comprising the steps forming water-absorbent polymer particles by polymerizing a monomer solution, coating of water-absorbent polymer particles with at least one surface-postcrosslinker and thermal surface-postcrosslinking of the coated water-absorbent polymer particles, wherein the content of residual monomers in the water-absorbent polymer particles prior to the coating with the surface-postcrosslinker is in the range from 0.1 to 10% by weight, the surface-postcrosslinker is an alkylene carbonate, and the temperature during the thermal surface-postcrosslinking is in the range from 100 to 180° C.

The level of residual monomers in the water-absorbent polymer particles prior to the thermal surface-postcrosslinking, the temperature of the thermal surface-postcrosslinking, and the surface-postcrosslinker itself have an important impact on the properties of the formed surface-postcrosslinked water-absorbent polymer particles.

The combination of having a high centrifuge retention capacity (CRC) and a high absorption under a load of 49.2 g/cm² (AUHL) results in water-absorbent polymer particles having a high total liquid uptake in the wicking absorption test.

The water-absorbent polymer particles further having a reduced pressure dependency of the characteristic swelling time in the VAUL test at high centrifuge retention capacities (CRC).

The water-absorbent polymer particles further having a level of extractable constituents of less than 10% by weight

Furthermore it is preferred that the surface-postcrosslinked water-absorbent polymer particles having a centrifuge retention capacity (CRC) from 35 to 75 g/g, an absorption under high load (AUHL) from 20 to 50 g/g, a level of extractable constituents of less than 10% by weight, and a porosity from 20 to 40%. The water-absorbent polymer particles having a high centrifuge retention capacity (CRC) and a high absorption under a load of 49.2 g/cm² (AUHL).

It is preferred that the water-absorbent polymer particles having a total liquid uptake of

Y>−500×ln(X)+1880

wherein Y [g] is the total liquid uptake and X [g/g] is the centrifuge retention capacity, wherein the centrifuge retention capacity is at least 25 g/g and the liquid uptake is at least 30 g.

Further suitable water-absorbent polymer particles having a change of characteristic swelling time of less than 0.6 and a centrifuge retention capacity of at least 35 g/g, wherein the change of characteristic swelling time is

Z<(τ_{0.5}−τ_{0.1})/τ_{0.5}

wherein Z is the change of characteristic swelling time, τ_{0.1} is the characteristic swelling time under a pressure of 0.1 psi (6.9 g/cm²) and τ_{0.5} is the characteristic swelling time under a pressure of 0.5 psi (35.0 g/cm²).

### DETAILED DESCRIPTION OF THE INVENTION

### A. Definitions

As used herein, the term “fluid-absorbent article” refers to any three-dimensional solid material being able to acquire and store fluids discharged from the body. Preferred fluid-absorbent articles are disposable fluid-absorbent articles that are designed to be worn in contact with the body of a user such as disposable fluid-absorbent pantyliners, incontinence inserts/pads, diapers, training pant diapers, breast pads, interlabial inserts/pads or other articles useful for absorbing body fluids.

As used herein, the term “fluid-absorbent composition” refers to a component of the fluid-absorbent article which is primarily responsible for the fluid handling of the fluid-absorbent article including acquisition, transport, distribution and storage of body fluids.

As used herein, the term “fluid-absorbent core” refers to a fluid-absorbent composition comprising water-absorbent polymer particles and a fibrous material. The fluid-absorbent core is primarily responsible for the fluid handling of the fluid-absorbent article including acquisition, transport, distribution and storage of body fluids.

As used herein, the term “layer” refers to a fluid-absorbent composition whose primary dimension is along its length and width. It should be known that the term “layer” is not necessarily limited to single layers or sheets of the fluid-absorbent composition. Thus a layer can comprise laminates, composites, combinations of several sheets or webs of different materials.

As used herein the term “x-dimension” refers to the length, and the term “y-dimension” refers to the width of the fluid-absorbent composition, layer, core or article. Generally, the term “x-y-dimension” refers to the plane, orthogonal to the height or thickness of the fluid-absorbent composition, layer, core or article.

As used herein the term “z-dimension” refers to the dimension orthogonal to the length and width of the fluid absorbent composition, layer, core or article. Generally, the term “z-dimension” refers to the height of the fluid-absorbent composition, layer, core or article.

As used herein, the term “basis weight” indicates the weight of the fluid-absorbent core per square meter or of the acquisition-distribution layer per square meter respectively. The basis weight is determined at discrete regions of the fluid-absorbent core or acquisition distribution layer respectively

Further, it should be understood, that the term “upper” refers to fluid-absorbent composition which are nearer to the wearer of the fluid-absorbent article. Generally, the topsheet is the nearest composition to the wearer of the fluid-absorbent article, hereinafter described as “upper liquid-pervious layer”. Contrarily, the term “lower” refers to fluid-absorbent compositions which are away from the wearer of the fluid-absorbent article. Generally, the backsheet is the component which is furthermost away from the wearer of the fluid-absorbent article, hereinafter described as “lower liquid-impervious layer”.

As used herein, the term “liquid-pervious” refers to a substrate, layer or a laminate thus permitting liquids, i.e. body fluids such as urine, menses and/or vaginal fluids to readily penetrate through its thickness.

As used herein, the term “liquid-impervious” refers to a substrate, layer or a laminate that does not allow body fluids to pass through in a direction generally perpendicular to the plane of the layer at the point of liquid contact under ordinary use conditions.

As used herein, the term “chassis” refers to fluid-absorbent material comprising the upper liquid-pervious layer and the lower liquid-impervious layer, elastication and closure systems for the absorbent article.

As used herein, the term “hydrophilic” refers to the wettability of fibers by water deposited on these fibers. The term “hydrophilic” is defined by the contact angle and surface tension of the body fluids. According to the definition of Robert F. Gould in the 1964 American Chemical Society publication “Contact angle, wettability and adhesion”, a fiber is referred to as hydrophilic, when the contact angle between the liquid and the fiber, especially the fiber surface, is less than 90° or when the liquid tends to spread spontaneously on the same surface.

Contrarily, term “hydrophobic” refers to fibers showing a contact angle of greater than 90° or no spontaneously spreading of the liquid across the surface of the fiber.

As used herein, the term “body fluids” refers to any fluid produced and discharged by human or animal body, such as urine, menstrual fluids, faeces, vaginal secretions and the like.

As used herein, the term “breathable” refers to a substrate, layer, film or a laminate that allows vapour to escape from the fluid-absorbent article, while still preventing fluids from leakage. Breathable substrates, layers, films or laminates may be porous polymeric films, nonwoven laminates from spunbond and melt-blown layers, laminates from porous polymeric films and nonwovens.

As used herein, the term “longitudinal” refers to a direction running perpendicular from a waist edge to an opposing waist edge of the fluid-absorbent article.

### B. Water-Absorbent Polymer Particles

The water-absorbent polymer particles are prepared by a process, comprising the steps forming water-absorbent polymer particles by polymerizing a monomer solution, comprising
a) at least one ethylenically unsaturated monomer which bears acid groups and may be at least partly neutralized,
b) optionally one or more cross-linker,
c) at least one initiator,
d) optionally one or more ethylenically unsaturated monomers copolymerizable with the monomers mentioned under a),
e) optionally one or more water-soluble polymers,
f) water, and
   optionally coating of water-absorbent polymer particles with at least one surface-postcrosslinker and thermal surface-postcrosslinking of the coated water-absorbent polymer particles, wherein the content of residual monomers in the water-absorbent polymer particles prior to the coating with the surface-postcrosslinker is in the range from 0.03 to 15% by weight, the surface-postcrosslinker is an alkylene carbonate, and the temperature during the thermal surface-postcrosslinking is in the range from 100 to 180° C.

The water-absorbent polymer particles are typically insoluble but swellable in water.

The monomers a) are preferably water-soluble, i.e. the solubility in water at 23° C. is typically at least 1 g/100 g of water, preferably at least 5 g/100 g of water, more preferably at least 25 g/100 g of water, most preferably at least 35 g/100 g of water.

Suitable monomers a) are, for example, ethylenically unsaturated carboxylic acids such as acrylic acid, methacrylic acid, maleic acid, and itaconic acid. Particularly preferred monomers are acrylic acid and methacrylic acid. Very particular preference is given to acrylic acid.

Further suitable monomers a) are, for example, ethylenically unsaturated sulfonic acids such as vinylsulfonic acid, styrenesulfonic acid and 2-acrylamido-2-methylpropanesulfonic acid (AMPS).

Impurities may have a strong impact on the polymerization. Preference is given to especially purified monomers a). Useful purification methods are disclosed in WO 2002/055469 A1, WO 2003/078378 A1 and WO 2004/035514 A1. A suitable monomer a) is according to WO 2004/035514 A1 purified acrylic acid having 99.8460% by weight of acrylic acid, 0.0950% by weight of acetic acid, 0.0332% by weight of water, 0.0203 by weight of propionic acid, 0.0001% by weight of furfurals, 0.0001% by weight of maleic anhydride, 0.0003% by weight of diacrylic acid and 0.0050% by weight of hydroquinone monomethyl ether.

Polymerized diacrylic acid is a source for residual monomers due to thermal decomposition. If the temperatures during the process are low, the concentration of diacrylic acid is no more critical and acrylic acids having higher concentrations of diacrylic acid, i.e. 500 to 10,000 ppm, can be used for the inventive process.

The content of acrylic acid and/or salts thereof in the total amount of monomers a) is preferably at least 50 mol %, more preferably at least 90 mol %, most preferably at least 95 mol %.

The acid groups of the monomers a) are typically partly neutralized in the range of 0 to 100 mol %, preferably to an extent of from 25 to 85 mol %, preferentially to an extent of from 50 to 80 mol %, more preferably from 60 to 75 mol %, for which the customary neutralizing agents can be used, preferably alkali metal hydroxides, alkali metal oxides, alkali metal carbonates or alkali metal hydrogen carbonates, and mixtures thereof. Instead of alkali metal salts, it is also possible to use ammonia or organic amines, for example, triethanolamine. It is also possible to use oxides, carbonates, hydrogencarbonates and hydroxides of magnesium, calcium, strontium, zinc or aluminum as powders, slurries or solutions and mixtures of any of the above neutralization agents. Example for a mixture is a solution of sodiumaluminate. Sodium and potassium are particularly preferred as alkali metals, but very particular preference is given to sodium hydroxide, sodium carbonate or sodium hydrogen carbonate, and mixtures thereof. Typically, the neutralization is achieved by mixing in the neutralizing agent as an aqueous solution, as a melt or preferably also as a solid. For example, sodium hydroxide with water content significantly below 50% by weight may be present as a waxy material having a melting point above 23° C. In this case, metered addition as piece material or melt at elevated temperature is possible.

Optionally, it is possible to add to the monomer solution, or to starting materials thereof, one or more chelating agents for masking metal ions, for example iron, for the purpose of stabilization. Suitable chelating agents are, for example, alkali metal citrates, citric acid, alkali metal tartrates, alkali metal lactates and glycolates, pentasodium triphosphate, ethylenediamine tetraacetate, nitrilotriacetic acid, and all chelating agents known under the Trilon® name, for example Trilon® C (pentasodium diethylenetriaminepentaacetate), Trilon® D (trisodium (hydroxyethyl)-ethylenediaminetriacetate), and Trilon® M (methylglycinediacetic acid).

The monomers a) comprise typically polymerization inhibitors, preferably hydroquinone monoethers, as inhibitor for storage.

The monomer solution comprises preferably up to 250 ppm by weight, more preferably not more than 130 ppm by weight, most preferably not more than 70 ppm by weight, preferably not less than 10 ppm by weight, more preferably not less than 30 ppm by weight and especially about 50 ppm by weight of hydroquinone monoether, based in each case on acrylic acid, with acrylic acid salts being counted as acrylic acid. For example, the monomer solution can be prepared using acrylic acid having appropriate hydroquinone monoether content. The hydroquinone monoethers may, however, also be removed from the monomer solution by absorption, for example on activated carbon.

Preferred hydroquinone monoethers are hydroquinone monomethyl ether (MEHQ) and/or alpha-tocopherol (vitamin E).

Suitable crosslinkers b) are compounds having at least two groups suitable for crosslinking. Such groups are, for example, ethylenically unsaturated groups which can be polymerized by a free-radical mechanism into the polymer chain and functional groups which can form covalent bonds with the acid groups of monomer a). In addition, polyvalent metal ions which can form coordinate bond with at least two acid groups of monomer a) are also suitable crosslinkers b).

The crosslinkers b) are preferably compounds having at least two free-radically polymerizable groups which can be polymerized by a free-radical mechanism into the polymer network. Suitable crosslinkers b) are, for example, ethylene glycol dimethacrylate, diethylene glycol diacrylate, polyethylene glycol diacrylate, allyl methacrylate, trimethylolpropane triacrylate, triallylamine, tetraallylammonium chloride, tetraallyloxyethane, as described in EP 0 530 438 A1, di- and triacrylates, as described in EP 0 547 847 A1, EP 0 559 476 A1, EP 0 632 068 A1, WO 93/21237 A1, WO 2003/104299 A1, WO 2003/104300 A1, WO 2003/104301 A1 and in DE 103 31 450 A1, mixed acrylates which, as well as acrylate groups, comprise further ethylenically unsaturated groups, as described in DE 103 314 56 A1 and DE 103 55 401 A1, or crosslinker mixtures, as described, for example, in DE 195 43 368 A1, DE 196 46 484 A1, WO 90/15830 A1 and WO 2002/32962 A2.

Suitable crosslinkers b) are in particular pentaerythritol triallyl ether, tetraallyloxyethane, polyethyleneglycole diallylethers (based on polyethylene glycole having a molecular weight between 400 and 20000 g/mol), N,N′-methylenebisacrylamide, 15-tuply ethoxylated trimethylolpropane, polyethylene glycol diacrylate, trimethylolpropane triacrylate and triallylamine.

Very particularly preferred crosslinkers b) are the polyethoxylated and/or -propoxylated glycerols which have been esterified with acrylic acid or methacrylic acid to give di- or triacrylates, as described, for example in WO 2003/104301 A1. Di- and/or triacrylates of 3- to 18-tuply ethoxylated glycerol are particularly advantageous. Very particular preference is given to di- or triacrylates of 1- to 5-tuply ethoxylated and/or propoxylated glycerol. Most preferred are the triacrylates of 3- to 5-tuply ethoxylated and/or propoxylated glycerol and especially the triacrylate of 3-tuply ethoxylated glycerol.

The amount of crosslinker b) is preferably from 0.0001 to 0.6% by weight, more preferably from 0.001 to 0.2% by weight, most preferably from 0.01 to 0.06% by weight, based in each case on monomer a). On increasing the amount of crosslinker b) the centrifuge retention capacity (CRC) decreases and the absorption under a pressure of 21.0 g/cm² (AUL) passes through a maximum.

The surface-postcrosslinked polymer particles of the present invention surprisingly require very little or even no cross-linker during the polymerization step. So, in one particularly preferred embodiment of the present invention no crosslinker b) is used.

The initiators c) used may be all compounds which disintegrate into free radicals under the polymerization conditions, for example peroxides, hydroperoxides, hydrogen peroxide, per-sulfates, azo compounds and redox initiators. Preference is given to the use of water-soluble initiators. In some cases, it is advantageous to use mixtures of various initiators, for example mixtures of hydrogen peroxide and sodium or potassium peroxodisulfate. Mixtures of hydrogen peroxide and sodium peroxodisulfate can be used in any proportion.

Particularly preferred initiators c) are azo initiators such as 2,2′-azobis[2-(2-imidazolin-2-yl)propane] dihydrochloride and 2,2′-azobis[2-(5-methyl-2-imidazolin-2-yl)propane] dihydrochloride, 2,2′-azobis(2-amidinopropane) dihydrochloride, 4,4′-azobis(4-cyanopentanoic acid), 4,4′-azobis(4-cyanopentanoic acid) sodium salt, 2,2′-azobis[2-methyl-N-(2-hydroxyethyl)propionamide], and photoinitiators such as 2-hydroxy-2-methylpropiophenone and 1-[4-(2-hydroxyethoxy)phenyl]-2-hydroxy-2-methyl-1-propan-1-one, redox initiators such as sodium persulfate/hydroxymethylsulfinic acid, ammonium peroxodisulfate/hydroxymethylsulfinic acid, hydrogen peroxide/hydroxymethylsulfinic acid, sodium per-sulfate/ascorbic acid, ammonium peroxodisulfate/ascorbic acid and hydrogen peroxide/ascorbic acid, photoinitiators such as 1-[4-(2-hydroxyethoxy)phenyl]-2-hydroxy-2-methyl-1-propan-1-one, and mixtures thereof. The reducing component used is, however, preferably a mixture of the sodium salt of 2-hydroxy-2-sulfinatoacetic acid, the disodium salt of 2-hydroxy-2-sulfonatoacetic acid and sodium bisulfite. Such mixtures are obtainable as Brüggolite® FF6 and Brüggolite® FF7 (Brüggemann Chemicals; Heilbronn; Germany). Of course it is also possible within the scope of the present invention to use the purified salts or acids of 2-hydroxy-2-sulfinatoacetic acid and 2-hydroxy-2-sulfonatoacetic acid—the latter being available as sodium salt under the trade name Blancolen® (Brüggemann Chemicals; Heilbronn; Germany).

The initiators are used in customary amounts, for example in amounts of from 0.001 to 5% by weight, preferably from 0.01 to 2% by weight, most preferably from 0.05 to 0.5% by weight, based on the monomers a).

Examples of ethylenically unsaturated monomers d) which are copolymerizable with the monomers a) are acrylamide, methacrylamide, hydroxyethyl acrylate, hydroxyethyl methacrylate, dimethylaminoethyl acrylate, dimethylaminoethyl methacrylate, dimethylaminopropyl acrylate and diethylaminopropyl methacrylate.

Useful water-soluble polymers e) include polyvinyl alcohol, modified polyvinyl alcohol comprising acidic side groups for example Poval® K (Kuraray Europe GmbH; Frankfurt; Germany), polyvinylpyrrolidone, starch, starch derivatives, modified cellulose such as methyl-cellulose, carboxymethylcellulose or hydroxyethylcellulose, gelatin, polyglycols or polyacrylic acids, polyesters and polyamides, polylactic acid, polyglycolic acid, co-polylactic-polyglycolic acid, polyvinylamine, polyallylamine, water soluble copolymers of acrylic acid and maleic acid available as Sokalan® (BASF SE; Ludwigshafen; Germany), preferably starch, starch derivatives and modified cellulose.

For optimal action, the preferred polymerization inhibitors require dissolved oxygen. Therefore, the monomer solution can be freed of dissolved oxygen before the polymerization by inertization, i.e. flowing through with an inert gas, preferably nitrogen. It is also possible to reduce the concentration of dissolved oxygen by adding a reducing agent. The oxygen content of the monomer solution is preferably lowered before the polymerization to less than 1 ppm by weight, more preferably to less than 0.5 ppm by weight.

The water content of the monomer solution is preferably less than 65% by weight, preferentially less than 62% by weight, more preferably less than 60% by weight, most preferably less than 58% by weight.

The monomer solution has, at 20° C., a dynamic viscosity of preferably from 0.002 to 0.02 Pa·s, more preferably from 0.004 to 0.015 Pa·s, most preferably from 0.005 to 0.01 Pa·s. The mean droplet diameter in the droplet generation rises with rising dynamic viscosity.

The monomer solution has, at 20° C., a density of preferably from 1 to 1.3 g/cm³, more preferably from 1.05 to 1.25 g/cm³, most preferably from 1.1 to 1.2 g/cm³.

The monomer solution has, at 20° C., a surface tension of from 0.02 to 0.06 N/m, more preferably from 0.03 to 0.05 N/m, most preferably from 0.035 to 0.045 N/m. The mean droplet diameter in the droplet generation rises with rising surface tension.

Polymerization

The monomer solution is polymerized. Suitable reactors are, for example, kneading reactors or belt reactors. In the kneader, the polymer gel formed in the polymerization of an aqueous monomer solution or suspension is comminuted continuously by, for example, contrarotatory stirrer shafts, as described in WO 2001/038402 A1. Polymerization on the belt is described, for example, in DE 38 25 366 A1 and U.S. Pat. No. 6,241,928. Polymerization in a belt reactor forms a polymer gel which has to be comminuted in a further process step, for example in an extruder or kneader.

To improve the drying properties, the comminuted polymer gel obtained by means of a kneader can additionally be extruded.

It is preferred to produce the water-absorbent polymer particles polymerizing droplets of the monomer in a surrounding heated gas phase, for example using a system described in WO 2008/040715 A2, WO 2008/052971 A1, WO 2008/069639 A1 and WO 2008/086976 A1.

The droplets are preferably generated by means of a droplet plate. A droplet plate is a plate having a multitude of bores, the liquid entering the bores from the top. The droplet plate or the liquid can be oscillated, which generates a chain of ideally monodisperse droplets at each bore on the underside of the droplet plate. In a preferred embodiment, the droplet plate is not agitated.

It is also possible to use two or more droplet plates with different bore diameters so that a range of desired particle sizes can be produced. It is preferable that each droplet plate carries only one bore diameter, however mixed bore diameters in one plate are also possible.

The number and size of the bores are selected according to the desired capacity and droplet size. The droplet diameter is typically 1.9 times the diameter of the bore. What is important here is that the liquid to be dropletized does not pass through the bore too rapidly and the pressure drop over the bore is not too great. Otherwise, the liquid is not dropletized, but rather the liquid jet is broken up (sprayed) owing to the high kinetic energy. The Reynolds number based on the throughput per bore and the bore diameter is preferably less than 2000, preferentially less than 1600, more preferably less than 1400 and most preferably less than 1200.

The underside of the droplet plate has at least in part a contact angle preferably of at least 60°, more preferably at least 75° and most preferably at least 90° with regard to water.

The contact angle is a measure of the wetting behavior of a liquid, in particular water, with regard to a surface, and can be determined using conventional methods, for example in accordance with ASTM D 5725. A low contact angle denotes good wetting, and a high contact angle denotes poor wetting.

It is also possible for the droplet plate to consist of a material having a lower contact angle with regard to water, for example a steel having the German construction material code number of 1.4571, and be coated with a material having a larger contact angle with regard to water.

Useful coatings include for example fluorous polymers, such as perfluoroalkoxyethylene, polytetrafluoroethylene, ethylene-chlorotrifluoroethylene copolymers, ethylene-tetrafluoroethylene copolymers and fluorinated polyethylene.

The coatings can be applied to the substrate as a dispersion, in which case the solvent is subsequently evaporated off and the coating is heat treated. For polytetrafluoroethylene this is described for example in U.S. Pat. No. 3,243,321.

Further coating processes are to be found under the headword “Thin Films” in the electronic version of “Ullmann's Encyclopedia of Industrial Chemistry” (Updated Sixth Edition, 2000 Electronic Release).

The coatings can further be incorporated in a nickel layer in the course of a chemical nickelization.

It is the poor wettability of the droplet plate that leads to the production of monodisperse droplets of narrow droplet size distribution.

The droplet plate has preferably at least 5, more preferably at least 25, most preferably at least 50 and preferably up to 750, more preferably up to 500 bores, most preferably up to 250. The number of bores is determined mainly by geometrical and manufacturing constraints and can be adjusted to practical use conditions even outside the above given range. The diameter of the bores is adjusted to the desired droplet size.

The separation of the bores is usually from 5 to 50 mm, preferably from 6 to 40 mm, more preferably from 7 to 35 mm, most preferably from 8 to 30 mm. Smaller separations of the bores may cause agglomeration of the polymerizing droplets.

The diameter of the bores is preferably from 50 to 500 μm, more preferably from 100 to 300 μm, most preferably from 150 to 250 μm.

For optimizing the average particle diameter, droplet plates with different bore diameters can be used. The variation can be done by different bores on one plate or by using different plates, where each plate has a different bore diameter. The average particle size distribution can be monomodal, bimodal or multimodal. Most preferably it is monomodal or bimodal.

The temperature of the monomer solution as it passes through the bore is preferably from 5 to 80° C., more preferably from 10 to 70° C., most preferably from 30 to 60° C.

A gas flows through the reaction chamber. The carrier gas is conducted through the reaction chamber in cocurrent to the free-falling droplets of the monomer solution, i.e. from the top downward. After one pass, the gas is preferably recycled at least partly, preferably to an extent of at least 50%, more preferably to an extent of at least 75%, into the reaction chamber as cycle gas. Typically, a portion of the carrier gas is discharged after each pass, preferably up to 10%, more preferably up to 3% and most preferably up to 1%.

The carrier gas may be composed of air. The oxygen content of the carrier gas is preferably from 0.1 to 15% by volume, more preferably from 1 to 10% by volume, most preferably from 2 to 7% by weight. In the scope of the present invention it is also possible to use a carrier gas which is free of oxygen.

As well as oxygen, the carrier gas preferably comprises nitrogen. The nitrogen content of the gas is preferably at least 80% by volume, more preferably at least 90% by volume, most preferably at least 95% by volume. Other possible carrier gases may be selected from carbon dioxide, argon, xenon, krypton, neon, helium, sulfurhexafluoride. Any mixture of carrier gases may be used. The carrier gas may also become loaded with water and/or acrylic acid vapors.

The gas velocity is preferably adjusted such that the flow in the reaction zone is directed, for example no convection currents opposed to the general flow direction are present, and is preferably from 0.1 to 2.5 m/s, more preferably from 0.3 to 1.5 m/s, even more preferably from 0.5 to 1.2 m/s, most preferably from 0.7 to 0.9 m/s.

The gas entrance temperature, i.e. the temperature with which the gas enters the reaction zone, is preferably from 160 to 200° C., more preferably from 165 to 195° C., even more preferably from 170 to 190° C., most preferably from 175 to 185° C.

The steam content of the gas that enters the reaction zone is preferably from 0.01 to 0.15 kg per kg dry gas, more preferably from 0.02 to 0.12 kg per kg dry gas, most preferably from 0.03 to 0.10 kg per kg dry gas.

The gas entrance temperature is controlled in such a way that the gas exit temperature, i.e. the temperature with which the gas leaves the reaction zone, is less than 150° C., preferably from 90 to 140° C., more preferably from 100 to 130° C., even more preferably from 105 to 125° C., most preferably from 110 to 120° C.

The steam content of the gas that leaves the reaction zone is preferably from 0.02 to 0.30 kg per kg dry gas, more from 0.04 to 0.28 kg per kg dry gas, most from 0.05 to 0.25 kg per kg dry gas.

The water-absorbent polymer particles can be divided into three categories: water-absorbent polymer particles of Type 1 are particles with one cavity, water-absorbent polymer particles of Type 2 are particles with more than one cavity, and water-absorbent polymer particles of Type 3 are solid particles with no visible cavity. Type 1 particles are represented by hollow-spheres, Type 2 particles are represented by spherical closed cell sponges, and Type 3 particles are represented by solid spheres. Type 2 or Type 3 particles or mixtures thereof with little or no Type 1 particles are preferred.

The morphology of the water-absorbent polymer particles can be controlled by the reaction conditions during polymerization. Water-absorbent polymer particles having a high amount of particles with one cavity (Type 1) can be prepared by using low gas velocities and high gas exit temperatures. Water-absorbent polymer particles having a high amount of particles with more than one cavity (Type 2) can be prepared by using high gas velocities and low gas exit temperatures.

Water-absorbent polymer particles having no cavity (Type 3) and water-absorbent polymer particles having more than one cavity (Type 2) show an improved mechanical stability compared with water-absorbent polymer particles having only one cavity (Type 1).

As a particular advantage round shaped particles have no edges that can easily be broken by processing stress in diaper production and during swelling in aqueous liquid there are no breakpoints on the surface that could lead to loss of mechanical strength.

The reaction can be carried out under elevated pressure or under reduced pressure, preferably from 1 to 100 mbar below ambient pressure, more preferably from 1.5 to 50 mbar below ambient pressure, most preferably from 2 to 10 mbar below ambient pressure.

The reaction off-gas, i.e. the gas leaving the reaction chamber, may be cooled in a heat exchanger. This condenses water and unconverted monomer a). The reaction off-gas can then be reheated at least partly and recycled into the reaction chamber as cycle gas. A portion of the reaction off-gas can be discharged and replaced by fresh gas, in which case water and unconverted monomers a) present in the reaction off-gas can be removed and recycled.

Particular preference is given to a thermally integrated system, i.e. a portion of the waste heat in the cooling of the off-gas is used to heat the cycle gas.

The reactors can be trace-heated. In this case, the trace heating is adjusted such that the wall temperature is at least 5° C. above the internal reactor temperature and condensation on the reactor walls is reliably prevented.

Thermal Posttreatment

The water-absorbent polymer particles obtained by dropletization may be thermal posttreated for adjusting the content of residual monomers to the desired value.

The residual monomers can be removed better at relatively high temperatures and relatively long residence times. What is important here is that the water-absorbent polymer particles are not too dry. In the case of excessively dry particles, the residual monomers decrease only insignificantly. Too high a water content increases the caking tendency of the water-absorbent polymer particles.

The thermal posttreatment can be done in a fluidized bed. In a preferred embodiment of the present invention an internal fluidized bed is used. An internal fluidized bed means that the product of the dropletization polymerization is accumulated in a fluidized bed below the reaction zone.

In the fluidized state, the kinetic energy of the polymer particles is greater than the cohesion or adhesion potential between the polymer particles.

The fluidized state can be achieved by a fluidized bed. In this bed, there is upward flow toward the water-absorbing polymer particles, so that the particles form a fluidized bed. The height of the fluidized bed is adjusted by gas rate and gas velocity, i.e. via the pressure drop of the fluidized bed (kinetic energy of the gas).

The velocity of the gas stream in the fluidized bed is preferably from 0.3 to 2.5 m/s, more preferably from 0.4 to 2.0 m/s, most preferably from 0.5 to 1.5 m/s.

The pressure drop over the bottom of the internal fluidized bed is preferably from 1 to 100 mbar, more preferably from 3 to 50 mbar, most preferably from 5 to 25 mbar.

The moisture content of the water-absorbent polymer particles at the end of the thermal posttreatment is preferably from 1 to 20% by weight, more preferably from 2 to 15% by weight, even more preferably from 3 to 12% by weight, most preferably 5 to 8% by weight.

The temperature of the water-absorbent polymer particles during the thermal posttreatment is from 20 to 120° C., preferably from 40 to 100° C., more preferably from 50 to 95° C., even more preferably from 55 to 90° C., most preferably from 60 to 80° C.

The average residence time in the internal fluidized bed is from 10 to 300 minutes, preferably from 60 to 270 minutes, more preferably from 40 to 250 minutes, most preferably from 120 to 240 minutes.

The condition of the fluidized bed can be adjusted for reducing the amount of residual monomers of the water-absorbent polymers leaving the fluidized bed. The amount of residual monomers can be reduced to levels below 0.1% by weight by a thermal posttreatment using additional steam.

The steam content of the gas is preferably from 0.005 to 0.25 kg per kg of dry gas, more preferably from 0.01 to 0.2 kg per kg of dry gas, most preferably from 0.02 to 0.15 kg per kg of dry gas.

By using additional steam the condition of the fluidized bed can be adjusted that the amount of residual monomers of the water-absorbent polymers leaving the fluidized bed is from 0.03 to 15% by weight, preferably from 0.05 to 12% by weight, more preferably from 0.1 to 10% by weight, even more preferably from 0.15 to 7.5% by weight most preferably from 0.2 to 5% by weight, even most preferably from 0.25 to 2.5% by weight.

The level of residual monomers in the water-absorbent polymer has an important impact on the properties of the later formed surface-postcrosslinked water-absorbent polymer particles. That means that very low levels of residual monomers must be avoided.

It is preferred that the thermal posttreatment is completely or at least partially done in an external fluidized bed. The operating conditions of the external fluidized bed are within the scope for the internal fluidized bed as described above.

It is alternatively preferred that the thermal posttreatment is done in an external mixer with moving mixing tools, preferably horizontal mixers, such as screw mixers, disk mixers, screw belt mixers and paddle mixers. Suitable mixers are, for example, Becker shovel mixers (Gebr. Lödige Maschinenbau GmbH; Paderborn; Germany), Nara paddle mixers (NARA Machinery Europe; Frechen; Germany), Pflugschar® plowshare mixers (Gebr. Lödige Maschinenbau GmbH; Paderborn; Germany), Vrieco-Nauta Continuous Mixers (Hosokawa Micron BV; Doetinchem; the Netherlands), Processall Mixmill Mixers (Processall Incorporated; Cincinnati; U.S.A.) and Ruberg continuous flow mixers (Gebrüder Ruberg GmbH & Co KG, Nieheim, Germany). Ruberg continuous flow mixers, Becker shovel mixers and Pflugschar® plowshare mixers are preferred.

The thermal posttreatment can be done in a discontinuous external mixer or a continuous external mixer.

The amount of gas to be used in the discontinuous external mixer is preferably from 0.01 to 5 Nm³/h, more preferably from 0.05 to 2 Nm³/h, most preferably from 0.1 to 0.5 Nm³/h, based in each case on kg water-absorbent polymer particles.

The amount of gas to be used in the continuous external mixer is preferably from 0.01 to 5 Nm³/h, more preferably from 0.05 to 2 Nm³/h, most preferably from 0.1 to 0.5 Nm³/h, based in each case on kg/h throughput of water-absorbent polymer particles.

The other constituents of the gas are preferably nitrogen, carbon dioxide, argon, xenon, krypton, neon, helium, air or air/nitrogen mixtures, more preferably nitrogen or air/nitrogen mixtures comprising less than 10% by volume of oxygen. Oxygen may cause discoloration.

The morphology of the water-absorbent polymer particles can also be controlled by the reaction conditions during thermal posttreatment. Water-absorbent polymer particles having a high amount of particles with one cavity (Type 1) can be prepared by using high product temperatures and short residence times. Water-absorbent polymer particles having a high amount of particles with more than one cavity (Type 2) can be prepared by using low product temperatures and long residence times.

Surface-Postcrosslinking

The polymer particles optionally can be surface-postcrosslinked for further improvement of the properties.

Surface-postcrosslinkers are compounds which comprise groups which can form at least two covalent bonds with the carboxylate groups of the polymer particles. Suitable compounds are, for example, polyfunctional amines, polyfunctional amidoamines, polyfunctional epoxides, as described in EP 0 083 022 A2, EP 0 543 303 A1 and EP 0 937 736 A2, di- or polyfunctional alcohols as described in DE 33 14 019 A1, DE 35 23 617 A1 and EP 0 450 922 A2, or β-hydroxyalkylamides, as described in DE 102 04 938 A1 and U.S. Pat. No. 6,239,230. Also ethyleneoxide, aziridine, glycidol, oxetane and its derivatives may be used.

Polyvinylamine, polyamidoamines and polyvinylalcohole are examples of multifunctional polymeric surface-postcrosslinkers.

In addition, DE 40 20 780 C1 describes alkylene carbonates, DE 198 07 502 A1 describes 1,3-oxazolidin-2-one and its derivatives such as 2-hydroxyethyl-1,3-oxazolidin-2-one, DE 198 07 992 C1 describes bis- and poly-1,3-oxazolidin-2-ones, EP 0 999 238 A1 describes bis- and poly-1,3-oxazolidines, DE 198 54 573 A1 describes 2-oxotetrahydro-1,3-oxazine and its derivatives, DE 198 54 574 A1 describes N-acyl-1,3-oxazolidin-2-ones, DE 102 04 937 A1 describes cyclic ureas, DE 103 34 584 A1 describes bicyclic amide acetals, EP 1 199 327 A2 describes oxetanes and cyclic ureas, and WO 2003/31482 A1 describes morpholine-2,3-dione and its derivatives, as suitable surface-postcrosslinkers.

In addition, it is also possible to use surface-postcrosslinkers which comprise additional polymerizable ethylenically unsaturated groups, as described in DE 37 13 601 A1.

The at least one surface-postcrosslinker is selected from alkylene carbonates, 1,3-oxazolidin-2-ones, bis- and poly-1,3-oxazolidin-2-ones, bis- and poly-1,3-oxazolidines, 2-oxotetrahydro-1,3-oxazines, N-acyl-1,3-oxazolidin-2-ones, cyclic ureas, bicyclic amide acetals, oxetanes, and morpholine-2,3-diones. Suitable surface-postcrosslinkers are ethylene carbonate, 3-methyl-1,3-oxazolidin-2-one, 3-methyl-3-oxethanmethanol, 1,3-oxazolidin-2-one, 3-(2-hydroxyethyl)-1,3-oxazolidin-2-one, 1,3-dioxan-2-one or a mixture thereof.

It is also possible to use any suitable mixture of surface-postcrosslinkers. It is particularly favorable to use mixtures of 1,3-dioxolan-2-on (ethylene carbonate) and 1,3-oxazolidin-2-ones. Such mixtures are obtainable by mixing and partly reacting of 1,3-dioxolan-2-on (ethylene carbonate) with the corresponding 2-amino-alcohol (e.g. 2-aminoethanol) and may comprise ethylene glycol from the reaction.

It is preferred that at least one alkylene carbonate is used as surface-postcrosslinker. Suitable alkylene carbonates are 1,3-dioxolan-2-on (ethylene carbonate), 4-methyl-1,3-dioxolan-2-on (propylene carbonate), 4,5-dimethyl-1,3-dioxolan-2-on, 4,4-dimethyl-1,3-dioxolan-2-on, 4-ethyl-1,3-dioxolan-2-on, 4-hydroxymethyl-1,3-dioxolan-2-on (glycerine carbonate), 1,3-dioxane-2-on (trimethylene carbonate), 4-methyl-1,3-dioxane-2-on, 4,6-dimethyl-1,3-dioxane-2-on and 1,3-dioxepan-2-on, preferably 1,3-dioxolan-2-on (ethylene carbonate) and 1,3-dioxane-2-on (trimethylene carbonate), most preferably 1,3-dioxolan-2-on (ethylene carbonate).

The amount of surface-postcrosslinker is preferably from 0.1 to 10% by weight, more preferably from 0.5 to 7.5% by weight, most preferably from 1 to 5% by weight, based in each case on the polymer.

The content of residual monomers in the water-absorbent polymer particles prior to the coating with the surface-postcrosslinker is in the range from 0.03 to 15% by weight, preferably from 0.05 to 12% by weight, more preferably from 0.1 to 10% by weight, even more preferably from 0.15 to 7.5% by weight, most preferably from 0.2 to 5% by weight, even most preferably from 0.25 to 2.5% by weight.

The moisture content of the water-absorbent polymer particles prior to the thermal surface-postcrosslinking is preferably from 1 to 20% by weight, more preferably from 2 to 15% by weight, most preferably from 3 to 10% by weight.

Polyvalent cations can be applied to the particle surface in addition to the surface-postcrosslinkers before, during or after the thermal surface-postcrosslinking.

The polyvalent cations usable in the process according to the invention are, for example, divalent cations such as the cations of zinc, magnesium, calcium, iron and strontium, trivalent cations such as the cations of aluminum, iron, chromium, rare earths and manganese, tetravalent cations such as the cations of titanium and zirconium, and mixtures thereof. Possible counterions are chloride, bromide, sulfate, hydrogensulfate, methanesulfate, carbonate, hydrogencarbonate, nitrate, hydroxide, phosphate, hydrogenphosphate, dihydrogenphosphate, glycophosphate and carboxylate, such as acetate, glycolate, tartrate, formiate, propionate, 3-hydroxypropionate, lactamide and lactate, and mixtures thereof. Aluminum sulfate, aluminum acetate, and aluminum lactate are preferred. Aluminum lactate is more preferred. Using the inventive process in combination with the use of aluminum lactate, water-absorbent polymer particles having an extremely high total liquid uptake at lower centrifuge retention capacities (CRC) can be prepared.

Apart from metal salts, it is also possible to use polyamines and/or polymeric amines as polyvalent cations. A single metal salt can be used as well as any mixture of the metal salts and/or the polyamines above.

Preferred polyvalent cations and corresponding anions are disclosed in WO 2012/045705 A1 and are expressly incorporated herein by reference. Preferred polyvinylamines are disclosed in WO 2004/024816 A1 and are expressly incorporated herein by reference.

The amount of polyvalent cation used is, for example, from 0.001 to 1.5% by weight, preferably from 0.005 to 1% by weight, more preferably from 0.02 to 0.8% by weight, based in each case on the polymer.

The addition of the polyvalent metal cation can take place prior, after, or cocurrently with the surface-postcrosslinking. Depending on the formulation and operating conditions employed it is possible to obtain a homogeneous surface coating and distribution of the polyvalent cation or an inhomogenous typically spotty coating. Both types of coatings and any mixes between them are useful within the scope of the present invention.

The surface-postcrosslinking is typically performed in such a way that a solution of the surface-postcrosslinker is sprayed onto the hydrogel or the dry polymer particles. After the spraying, the polymer particles coated with the surface-postcrosslinker are dried thermally and cooled.

The spraying of a solution of the surface-postcrosslinker is preferably performed in mixers with moving mixing tools, such as screw mixers, disk mixers and paddle mixers. Suitable mixers are, for example, vertical Schugi Flexomix® mixers (Hosokawa Micron BV; Doetinchem; the Netherlands), Turbolizers® mixers (Hosokawa Micron BV; Doetinchem; the Netherlands), horizontal Pflugschar® plowshare mixers (Gebr. Lödige Maschinenbau GmbH; Paderborn; Germany), Vrieco-Nauta Continuous Mixers (Hosokawa Micron BV; Doetinchem; the Netherlands), Processall Mixmill Mixers (Processall Incorporated; Cincinnati; US) and Ruberg continuous flow mixers (Gebrüder Ruberg GmbH & Co KG, Nieheim, Germany). Ruberg continuous flow mixers and horizontal Pflugschar® plowshare mixers are preferred. The surface-postcrosslinker solution can also be sprayed into a fluidized bed.

The solution of the surface-postcrosslinker can also be sprayed on the water-absorbent polymer particles during the thermal posttreatment. In such case the surface-postcrosslinker can be added as one portion or in several portions along the axis of thermal posttreatment mixer. In one embodiment it is preferred to add the surface-postcrosslinker at the end of the thermal posttreatment step. As a particular advantage of adding the solution of the surface-postcrosslinker during the thermal posttreatment step it may be possible to eliminate or reduce the technical effort for a separate surface-postcrosslinker addition mixer.

The surface-postcrosslinkers are typically used as an aqueous solution. The addition of nonaqueous solvent can be used to improve surface wetting and to adjust the penetration depth of the surface-postcrosslinker into the polymer particles.

The thermal surface-postcrosslinking is preferably carried out in contact dryers, more preferably paddle dryers, most preferably disk dryers. Suitable driers are, for example, Hosokawa Bepex® horizontal paddle driers (Hosokawa Micron GmbH; Leingarten; Germany), Hosokawa Bepex® disk driers (Hosokawa Micron GmbH; Leingarten; Germany), Holo-Flite® dryers (Metso Minerals Industries Inc.; Danville; U.S.A.) and Nara paddle driers (NARA Machinery Europe; Frechen; Germany). Moreover, it is also possible to use fluidized bed dryers. In the latter case the reaction times may be shorter compared to other embodiments.

When a horizontal dryer is used then it is often advantageous to set the dryer up with an inclined angle of a few degrees vs. the earth surface in order to impart proper product flow through the dryer. The angle can be fixed or may be adjustable and is typically between 0 to 10 degrees, preferably 1 to 6 degrees, most preferably 2 to 4 degrees.

A contact dryer can be used that has two different heating zones in one apparatus. For example Nara paddle driers are available with just one heated zone or alternatively with two heated zones. The advantage of using a two or more heated zone dryer is that different phases of the thermal post-treatment and/or of the post-surface-crosslinking can be combined.

It is possible to use a contact dryer with a hot first heating zone which is followed by a temperature holding zone in the same dryer. This set up allows a quick rise of the product temperature and evaporation of surplus liquid in the first heating zone, whereas the rest of the dryer is just holding the product temperature stable to complete the reaction.

It is also possible to use a contact dryer with a warm first heating zone which is then followed by a hot heating zone. In the first warm zone the thermal post-treatment is affected or completed whereas the surface-postcrosslinking takes place in the subsequential hot zone.

Typically a paddle heater with just one temperature zone is employed.

A person skilled in the art will depending on the desired finished product properties and the available base polymer qualities from the polymerization step choose any one of these set ups.

The thermal surface-postcrosslinking can be effected in the mixer itself, by heating the jacket, blowing in warm air or steam. Equally suitable is a downstream dryer, for example a shelf dryer, a rotary tube oven or a heatable screw. It is particularly advantageous to mix and dry in a fluidized bed dryer.

Preferred thermal surface-postcrosslinking temperatures are in the range from 100 to 180° C., preferably from 120 to 170° C., more preferably from 130 to 165° C., most preferably from 140 to 160° C. The preferred residence time at this temperature in the reaction mixer or dryer is preferably at least 5 minutes, more preferably at least 20 minutes, most preferably at least 40 minutes, and typically at most 120 minutes.

It is preferable to cool the polymer particles after thermal surface-postcrosslinking. The cooling is preferably carried out in contact coolers, more preferably paddle coolers, most preferably disk coolers. Suitable coolers are, for example, Hosokawa Bepex® horizontal paddle coolers (Hosokawa Micron GmbH; Leingarten; Germany), Hosokawa Bepex® disk coolers (Hosokawa Micron GmbH; Leingarten; Germany), Holo-Flite® coolers (Metso Minerals Industries Inc.; Danville; U.S.A.) and Nara paddle coolers (NARA Machinery Europe; Frechen; Germany). Moreover, it is also possible to use fluidized bed coolers.

In the cooler the polymer particles are cooled to temperatures in the range from 20 to 150° C., preferably from 40 to 120° C., more preferably from 60 to 100° C., most preferably from 70 to 90° C. Cooling using warm water is preferred, especially when contact coolers are used.

Coating

To improve the properties, the water-absorbent polymer particles can be coated and/or optionally moistened. The internal fluidized bed, the external fluidized bed and/or the external mixer used for the thermal posttreatment and/or a separate coater (mixer) can be used for coating of the water-absorbent polymer particles. Further, the cooler and/or a separate coater (mixer) can be used for coating/moistening of the surface-postcrosslinked water-absorbent polymer particles. Suitable coatings for controlling the acquisition behavior and improving the permeability (SFC or GBP) are, for example, inorganic inert substances, such as water-insoluble metal salts, organic polymers, cationic polymers, anionic polymers and polyvalent metal cations. Suitable coatings for improving the color stability are, for example reducing agents, chelating agents and anti-oxidants. Suitable coatings for dust binding are, for example, polyols. Suitable coatings against the undesired caking tendency of the polymer particles are, for example, fumed silica, such as Aerosil® 200, and surfactants, such as Span® 20 and Plantacare® 818 UP. Preferred coatings are aluminium dihydroxy monoacetate, aluminium sulfate, aluminium lactate, aluminium 3-hydroxypropionate, zirconium acetate, citric acid or its water soluble salts, di- and mono-phosphoric acid or their water soluble salts, Blancolen®, Brüggolite® FF7, Cublen®, Span® 20 and Plantacare® 818 UP.

If salts of the above acids are used instead of the free acids then the preferred salts are alkali-metal, earth alkali metal, aluminum, zirconium, titanium, zinc and ammonium salts.

Under the trade name Cublen® (Zschimmer & Schwarz Mohsdorf GmbH & Co KG; Burgstadt; Germany) the following acids and/or their alkali metal salts (preferably Na and K-salts) are available and may be used within the scope of the present invention for example to impart color-stability to the finished product:

1-Hydroxyethane-1,1-diphosphonic acid, Amino-tris(methylene phosphonic acid), Ethylenediamine-tetra(methylene phosphonic acid), Diethylenetriamine-penta(methylene phosphonic acid), Hexamethylene diamine-tetra(methylenephosphonic acid), Hydroxyethyl-amino-di(methylene phosphonic acid), 2-Phosphonobutane-1,2,4-tricarboxylic acid, Bis(hexamethylenetriamine penta(methylene phosphonic acid).

Most preferably 1-Hydroxyethane-1,1-diphosphonic acid or its salts with sodium, potassium, or ammonium are employed. Any mixture of the above Cublenes® can be used.

Alternatively, any of the chelating agents described before for use in the polymerization can be coated onto the finished product.

Suitable inorganic inert substances are silicates such as montmorillonite, kaolinite and talc, zeolites, activated carbons, polysilicic acids, magnesium carbonate, calcium carbonate, calcium phosphate, aluminum phosphate, barium sulfate, aluminum oxide, titanium dioxide and iron(II) oxide. Preference is given to using polysilicic acids, which are divided between precipitated silicas and fumed silicas according to their mode of preparation. The two variants are commercially available under the names Silica FK, Sipernat®, Wessalon® (precipitated silicas) and Aerosil® (fumed silicas) respectively. The inorganic inert substances may be used as dispersion in an aqueous or water-miscible dispersant or in substance.

When the water-absorbent polymer particles are coated with inorganic inert substances, the amount of inorganic inert substances used, based on the water-absorbent polymer particles, is preferably from 0.05 to 5% by weight, more preferably from 0.1 to 1.5% by weight, most preferably from 0.3 to 1% by weight.

Suitable organic polymers are polyalkyl methacrylates or thermoplastics such as polyvinyl chloride, waxes based on polyethylene, polypropylene, polyamides or polytetrafluoroethylene. Other examples are styrene-isoprene-styrene block-copolymers or styrene-butadiene-styrene block-copolymers. Another example are silanole-group bearing polyvinylalcoholes available under the trade name Poval® R (Kuraray Europe GmbH; Frankfurt; Germany).

Suitable cationic polymers are polyalkylenepolyamines, cationic derivatives of polyacrylamides, polyethyleneimines and polyquaternary amines.

Polyquaternary amines are, for example, condensation products of hexamethylenediamine, dimethylamine and epichlorohydrin, condensation products of dimethylamine and epichlorohydrin, copolymers of hydroxyethylcellulose and diallyldimethylammonium chloride, copolymers of acrylamide and α-methacryloyloxyethyltrimethylammonium chloride, condensation products of hydroxyethylcellulose, epichlorohydrin and trimethylamine, homopolymers of diallyldimethylammonium chloride and addition products of epichlorohydrin to amidoamines. In addition, polyquaternary amines can be obtained by reacting dimethyl sulfate with polymers such as polyethyleneimines, copolymers of vinylpyrrolidone and dimethylaminoethyl methacrylate or copolymers of ethyl methacrylate and diethylaminoethyl methacrylate. The polyquaternary amines are available within a wide molecular weight range.

However, it is also possible to generate the cationic polymers on the particle surface, either through reagents which can form a network with themselves, such as addition products of epichlorohydrin to polyamidoamines, or through the application of cationic polymers which can react with an added crosslinker, such as polyamines or polyimines in combination with polyepoxides, polyfunctional esters, polyfunctional acids or polyfunctional (meth)acrylates.

It is possible to use all polyfunctional amines having primary or secondary amino groups, such as polyethyleneimine, polyallylamine and polylysine. The liquid sprayed by the process according to the invention preferably comprises at least one polyamine, for example polyvinylamine or a partially hydrolyzed polyvinylformamide.

The cationic polymers may be used as a solution in an aqueous or water-miscible solvent, as dispersion in an aqueous or water-miscible dispersant or in substance.

When the water-absorbent polymer particles are coated with a cationic polymer, the use amount of cationic polymer based on the water-absorbent polymer particles is usually not less than 0.001% by weight, typically not less than 0.01% by weight, preferably from 0.1 to 15% by weight, more preferably from 0.5 to 10% by weight, most preferably from 1 to 5% by weight.

Suitable anionic polymers are polyacrylates (in acidic form or partially neutralized as salt), copolymers of acrylic acid and maleic acid available under the trade name Sokalan® (BASF SE; Ludwigshafen; Germany), and polyvinylalcohols with built in ionic charges available under the trade name Poval® K (Kuraray Europe GmbH; Frankfurt; Germany).

Suitable polyvalent metal cations are Mg²⁺, Ca²⁺, Al³⁺, Sc³⁺, Ti⁴⁺, Mn²⁺, Fe^{2+/3+}, Co²⁺, Ni²⁺, Cu^{+/2}+, Zn²⁺, Y³⁺, Zr⁴⁺, Ag⁺, La³⁺, Ce⁴⁺, Hf⁴⁺ and Au^{+/3}+; preferred metal cations are Mg²⁺, Ca²⁺, Al³⁺, Ti⁴⁺, Zr⁴⁺ and La³⁺; particularly preferred metal cations are Al³⁺, Ti⁴⁺ and Zr⁴⁺. The metal cations may be used either alone or in a mixture with one another. Suitable metal salts of the metal cations mentioned are all of those which have a sufficient solubility in the solvent to be used. Particularly suitable metal salts have weakly complexing anions, such as chloride, hydroxide, carbonate, acetate, formiate, propionate, nitrate, sulfate and methanesulfate. The metal salts are preferably used as a solution or as a stable aqueous colloidal dispersion. The solvents used for the metal salts may be water, alcohols, ethylenecarbonate, propylenecarbonate, dimethylformamide, dimethyl sulfoxide and mixtures thereof. Particular preference is given to water and water/alcohol mixtures, such as water/methanol, water/isopropanol, water/1,3-propanediole, water/1,2-propandiole/1,4-butanediole or water/propylene glycol.

When the water-absorbent polymer particles are coated with a polyvalent metal cation, the amount of polyvalent metal cation used, based on the water-absorbent polymer particles, is preferably from 0.05 to 5% by weight, more preferably from 0.1 to 1.5% by weight, most preferably from 0.3 to 1% by weight.

Suitable reducing agents are, for example, sodium sulfite, sodium hydrogensulfite (sodium bisulfite), sodium dithionite, sulfinic acids and salts thereof, ascorbic acid, sodium hypophosphite, sodium phosphite, and phosphinic acids and salts thereof. Preference is given, however, to salts of hypophosphorous acid, for example sodium hypophosphite, salts of sulfinic acids, for example the disodium salt of 2-hydroxy-2-sulfinatoacetic acid, and addition products of aldehydes, for example the disodium salt of 2-hydroxy-2-sulfonatoacetic acid. The reducing agent used can be, however, a mixture of the sodium salt of 2-hydroxy-2-sulfinatoacetic acid, the disodium salt of 2-hydroxy-2-sulfonatoacetic acid and sodium bisulfite. Such mixtures are obtainable as Brüggolite® FF6 and Brüggolite® FF7 (Brüggemann Chemicals; Heilbronn; Germany). Also useful is the purified 2-hydroxy-2-sulfonatoacetic acid and its sodium salts, available under the trade name Blancolen® from the same company.

The reducing agents are typically used in the form of a solution in a suitable solvent, preferably water. The reducing agent may be used as a pure substance or any mixture of the above reducing agents may be used.

When the water-absorbent polymer particles are coated with a reducing agent, the amount of reducing agent used, based on the water-absorbent polymer particles, is preferably from 0.01 to 5% by weight, more preferably from 0.05 to 2% by weight, most preferably from 0.1 to 1% by weight.

Suitable polyols are polyethylene glycols having a molecular weight of from 400 to 20000 g/mol, polyglycerol, 3- to 100-tuply ethoxylated polyols, such as trimethylolpropane, glycerol, sorbitol, mannitol, inositol, pentaerythritol and neopentyl glycol. Particularly suitable polyols are 7- to 20-tuply ethoxylated glycerol or trimethylolpropane, for example Polyol TP 70® (Perstorp AB, Perstorp, Sweden). The latter have the advantage in particular that they lower the surface tension of an aqueous extract of the water-absorbent polymer particles only insignificantly. The polyols are preferably used as a solution in aqueous or water-miscible solvents.

The polyol can be added before, during, or after surface-crosslinking. Preferably it is added after surface-cross linking. Any mixture of the above listed poyols may be used.

When the water-absorbent polymer particles are coated with a polyol, the use amount of polyol, based on the water-absorbent polymer particles, is preferably from 0.005 to 2% by weight, more preferably from 0.01 to 1% by weight, most preferably from 0.05 to 0.5% by weight.

The coating is preferably performed in mixers with moving mixing tools, such as screw mixers, disk mixers, paddle mixers and drum coater. Suitable mixers are, for example, horizontal Pflugschar® plowshare mixers (Gebr. Lödige Maschinenbau GmbH; Paderborn; Germany), Vrieco-Nauta Continuous Mixers (Hosokawa Micron BV; Doetinchem; the Netherlands), Processall Mixmill Mixers (Processall Incorporated; Cincinnati; US) and Ruberg continuous flow mixers (Gebrüder Ruberg GmbH & Co KG, Nieheim, Germany). Moreover, it is also possible to use a fluidized bed for mixing.

Agglomeration

The water-absorbent polymer particles can further selectively be agglomerated. The agglomeration can take place after the polymerization, the thermal postreatment, the thermal surface-postcrosslinking or the coating.

Useful agglomeration assistants include water and water-miscible organic solvents, such as alcohols, tetrahydrofuran and acetone; water-soluble polymers can be used in addition.

For agglomeration a solution comprising the agglomeration assistant is sprayed onto the water-absorbing polymeric particles. The spraying with the solution can, for example, be carried out in mixers having moving mixing implements, such as screw mixers, paddle mixers, disk mixers, plowshare mixers and shovel mixers. Useful mixers include for example Lödige® mixers, Bepex® mixers, Nauta® mixers, Processall® mixers and Schugi® mixers.

Vertical mixers are preferred. Fluidized bed apparatuses are particularly preferred.

Combination of thermal posttreatment, surface-postcrosslinking and optionally coating

It is preferred that the steps of thermal posttreatment and thermal surface-postcrosslinking are combined in one process step. Such combination allows the use of low cost equipment and moreover the process can be run at low temperatures, that is cost-efficient and avoids discoloration and loss of performance properties of the finished product by thermal degradation.

The mixer may be selected from any of the equipment options cited in the thermal post-treatment section. Ruberg continuous flow mixers, Becker shovel mixers and Pflugschar® plowshare mixers are preferred.

It is particular preferred that the surface-postcrosslinking solution is sprayed onto the water-absorbent polymer particles under agitation.

Following the thermal posttreatment/surface-postcrosslinking the water-absorbent polymer particles are dried to the desired moisture level and for this step any dryer cited in the surface-postcrosslinking section may be selected. However, as only drying needs to be accomplished in this particular preferred embodiment it is possible to use simple and low cost heated contact dryers like a heated screw dryer, for example a Holo-Flite® dryer (Metso

Minerals Industries Inc.; Danville; U.S.A.). Alternatively a fluidized bed may be used. In cases where the product needs to be dried with a predetermined and narrow residence time it is possible to use torus disc dryers or paddle dryers, for example a Nara paddle dryer (NARA Machinery Europe; Frechen; Germany).

In a preferred embodiment of the present invention, polyvalent cations cited in the surface-postcrosslinking section are applied to the particle surface before, during or after addition of the surface-postcrosslinker by using different addition points along the axis of a horizontal mixer.

It is very particular preferred that the steps of thermal post-treatment, surface-postcrosslinking, and coating are combined in one process step. Suitable coatings are cationic polymers, surfactants, and inorganic inert substances that are cited in the coating section. The coating agent can be applied to the particle surface before, during or after addition of the surface-postcrosslinker also by using different addition points along the axis of a horizontal mixer.

The polyvalent cations and/or the cationic polymers can act as additional scavengers for residual surface-postcrosslinkers. It is preferred that the surface-postcrosslinkers are added prior to the polyvalent cations and/or the cationic polymers to allow the surface-postcrosslinker to react first.

The surfactants and/or the inorganic inert substances can be used to avoid sticking or caking during this process step under humid atmospheric conditions. Preferred surfactants are non-ionic and amphoteric surfactants. Preferred inorganic inert substances are precipitated silicas and fumed silicas in form of powder or dispersion.

The amount of total liquid used for preparing the solutions/dispersions is typically from 0.01% to 25% by weight, preferably from 0.5% to 12% by weight, more preferably from 2% to 7% by weight, most preferably from 3% to 6% by weight, in respect to the weight amount of water-absorbent polymer particles to be processed.

The surface-postcrosslinked water-absorbent polymer particles having a centrifuge retention capacity from 35 to 75 g/g, an absorption under high load from 20 to 50 g/g, a level of extractable constituents of less than 10% by weight, and a porosity from 20 to 40%.

It is particular advantageous that the surface-postcrosslinked water-absorbent polymer particles exhibit a very high centrifuge retention capacity (CRC) and a high absorption under high load (AUHL), and that the sum of these parameters (=CRC+AUHL) is at least 60 g/g, preferably at least 65 g/g, most preferably at least 70 g/g, and not more than 120 g/g, preferably less than 100 g/g, more preferably less than 90 g/g, and most preferably less than 80 g/g. The surface-postcrosslinked water-absorbent polymer particles further preferably exhibit an absorption under high load (AUHL) of at least 15 g/g, preferably at least 18 g/g, more preferably at least 21 g/g, most preferably at least 25 g/g, and not more than 50 g/g.

As the centrifuge retention capacity (CRC) is the maximum water retention capacity of the surface-postcrosslinked water-absorbent polymer particles it is of interest to maximize this parameter. However the absorption under high load (AUHL) is important to allow the fiber-matrix in a hygiene article to open up pores during swelling to allow further liquid to pass easily through the article structure to enable rapid uptake of this liquid. Hence there is a need to maximize both parameters.

The water-absorbent polymer particles have a centrifuge retention capacity (CRC) from 35 to 75 g/g, preferably from 37 to 65 g/g, more preferably from 39 to 60 g/g, most preferably from 40 to 55 g/g.

The water-absorbent polymer particles have an absorbency under a load of 49.2 g/cm² (AUHL) from 20 to 50 g/g, preferably from 22 to 45 g/g, more preferably from 24 to 40 g/g, most preferably from 25 to 35 g/g.

The water-absorbent polymer particles have a level of extractable constituents of less than 10% by weight, preferably less than 8% by weight, more preferably less than 6% by weight, most preferably less than 5% by weight.

The water-absorbent polymer particles have a porosity from 20 to 40%, preferably from 22 to 38%, more preferably from 24 to 36%, most preferably from 25 to 35%.

Preferred water-absorbent polymer particles are polymer particles having a centrifuge retention capacity (CRC) from 37 to 65 g/g, an absorption under high load (AUHL) from 22 to 45 g/g, a level of extractable constituents of less than 8% by weight and a porosity from 22 to 45%.

More preferred water-absorbent polymer particles are polymer particles having a centrifuge retention capacity (CRC) from 39 to 60 g/g, an absorption under high load (AUHL) from 24 to 40 g/g, a level of extractable constituents of less than 6% by weight and a porosity from 24 to 40%.

Most preferred water-absorbent polymer particles are polymer particles having a centrifuge retention capacity (CRC) from 40 to 55 g/g, an absorption under high load (AUHL) from 25 to 35 g/g, a level of extractable constituents of less than 5% by weight and a porosity from 25 to 35%.

Also preferred are surface-postcrosslinked water-absorbent polymer particles having a total liquid uptake of

Y>−500×ln(X)+1880,

preferably Y>−495×ln(X)+1875,

more preferably Y>−490×ln(X)+1870,

most preferably Y>−485×ln(X)+1865,

wherein Y [g] is the total liquid uptake and X [g/g] is the centrifuge retention capacity (CRC), wherein the centrifuge retention capacity (CRC) is at least 25 g/g, preferably at least 30 g/g, more preferably at least 35 g/g, most preferably at least 40 g/g, and the liquid uptake is at least 30 g, preferably at least 35 g/g, more preferably at least 40 g/g, most preferably at least 45 g/g.

Further suited are surface-postcrosslinked water-absorbent polymer particles having a change of characteristic swelling time of less than 0.6, preferably less than 0.5, more preferably less than 0.45, most preferably less than 0.4, and a centrifuge retention capacity (CRC) of at least 35 g/g, preferably at least 37 g/g, more preferably at least 38.5 g/g, most preferably at least 40 g/g, wherein the change of characteristic swelling time is

Z<(τ_{0.5}−τ_{0.1})/τ_{0.5}

wherein Z is the change of characteristic swelling time, τ_{0.1} is the characteristic swelling time under a pressure of 0.1 psi (6.9 g/cm²) and τ_{0.5} is the characteristic swelling time under a pressure of 0.5 psi (35.0 g/cm²).

The water-absorbent polymer particles suited for the present invention have a mean sphericity from 0.80 to 0.95, preferably from 0.82 to 0.93, more preferably from 0.84 to 0.91, most preferably from 0.85 to 0.90. The sphericity (SPHT) is defined as

$$SPHT = \frac{4 π A}{{U}^{2}} ,$$

where A is the cross-sectional area and U is the cross-sectional circumference of the polymer particles. The mean sphericity is the volume-average sphericity.

The mean sphericity can be determined, for example, with the Camsizer® image analysis system (Retsch Technology GmbH; Haan; Germany):

For the measurement, the product is introduced through a funnel and conveyed to the falling shaft with a metering channel. While the particles fall past a light wall, they are recorded selectively by a camera. The images recorded are evaluated by the software in accordance with the parameters selected.

To characterize the roundness, the parameters designated as sphericity in the program are employed. The parameters reported are the mean volume-weighted sphericities, the volume of the particles being determined via the equivalent diameter xcₘᵢₙ. To determine the equivalent diameter xcₘᵢₙ, the longest chord diameter for a total of 32 different spatial directions is measured in each case. The equivalent diameter xcₘᵢₙ is the shortest of these 32 chord diameters. To record the particles, the so-called CCD-zoom camera (CAM-Z) is used. To control the metering channel, a surface coverage fraction in the detection window of the camera (transmission) of 0.5% is predefined.

Water-absorbent polymer particles with relatively low sphericity are obtained by reverse suspension polymerization when the polymer beads are agglomerated during or after the polymerization.

The water-absorbent polymer particles prepared by customary solution polymerization (gel polymerization) are ground and classified after drying to obtain irregular polymer particles. The mean sphericity of these polymer particles is between approx. 0.72 and approx. 0.78.

Water-absorbent polymer particles suited for the present invention have a content of hydrophobic solvent of preferably less than 0.005% by weight, more preferably less than 0.002% by weight and most preferably less than 0.001% by weight. The content of hydrophobic solvent can be determined by gas chromatography, for example by means of the headspace technique. A hydrophobic solvent within the scope of the present invention is either immiscible in water or only sparingly miscible. Typical examples of hydrophobic solvents are pentane, hexane, cyclohexane, toluene.

Water-absorbent polymer particles which have been obtained by reverse suspension polymerization still comprise typically approx. 0.01% by weight of the hydrophobic solvent used as the reaction medium.

The water-absorbent polymer particles useful for the present invention have a dispersant content of typically less than 1% by weight, preferably less than 0.5% by weight, more preferably less than 0.1% by weight and most preferably less than 0.05% by weight.

Water-absorbent polymer particles which have been obtained by reverse suspension polymerization still comprise typically at least 1% by weight of the dispersant, i.e. ethylcellulose, used to stabilize the suspension.

Suitable water-absorbent polymer particles have a bulk density preferably from 0.6 to 1 g/cm³, more preferably from 0.65 to 0.9 g/cm³, most preferably from 0.68 to 0.8 g/cm³.

The average particle diameter (APD) of the water-absorbent particles useful for the present invention is preferably from 200 to 550 μm, more preferably from 250 to 500 μm, most preferably from 350 to 450 μm.

The particle diameter distribution (PDD) of the useful water-absorbent particles is preferably less than 0.7, more preferably less than 0.65, more preferably less than 0.6.

One kind of water-absorbent polymer particles can be mixed with other water-absorbent polymer particles prepared by other processes, i.e. solution polymerization.

### C. Fluid-Absorbent Articles

The fluid-absorbent article comprises of
(A) an upper liquid-pervious layer
(B) a lower liquid-impervious layer
(C) At least 50% by weight fluid-absorbent polymer particles and not more than 50% by weight of cellulose based fibers,
preferably at least 60% by weight fluid-absorbent polymer particles and not more than 40% by weight of cellulose based fibers,
more preferably at least 70% by weight fluid-absorbent polymer particles and not more than 30% by weight of cellulose based fibers,
most preferably at least 90% by weight fluid-absorbent polymer particles and not more than 10% by weight of cellulose based fibers, based on the sum of fluid-absorbent polymer particles and cellulose based fibres, and
(D) an acquisition-distribution layer between (A) and (C) having a three dimensional apertured structure having a femal side (5) and a male side (6), wherein the femal side presents a multiplicity of openings extending in the form of through holes in the direction of a lower surface of the same structure being the male side,
(E) an optional tissue layer disposed immediately above and/or below (C) or wrapped fully or partially around (C); and
(F) other optional components.

Fluid-absorbent articles are understood to mean, for example, incontinence pads and incontinence briefs for adults or diapers and training pants for babies. Suitable fluid-absorbent articles including fluid-absorbent compositions comprising fibrous materials and optionally water-absorbent polymer particles to form fibrous webs or matrices for the substrates, layers, sheets and/or the fluid-absorbent core.

The acquisition-distribution layer acts as transport and distribution layer of the discharged body fluids and is typically optimized to affect efficient liquid distribution with the underlying fluid-absorbent core. Hence, for quick temporary liquid retention it provides the necessary void space while its area coverage of the underlying fluid-absorbent core must affect the necessary liquid distribution and is adopted to the ability of the fluid-absorbent core to quickly dewater the acquisition-distribution layer.

Suitable fluid-absorbent articles are composed of several layers whose individual elements must show preferably definite functional parameter such as dryness for the upper liquid-pervious layer, vapor permeability without wetting through for the lower liquid-impervious layer, a flexible, vapor permeable and thin fluid-absorbent core, showing fast absorption rates and being able to retain highest quantities of body fluids, and an acquisition-distribution layer between the upper layer and the core, acting as transport and distribution layer of the discharged body fluids. These individual elements are combined such that the resultant fluid-absorbent article meets overall criteria such as flexibility, water vapour breathability, dryness, wearing comfort and protection on the user facing side, and concerning liquid retention, rewet and prevention of wet through on the garment side. The specific combination of these layers provides a fluid-absorbent article delivering both high protection levels as well as high comfort to the consumer.

Designs for fluid-absorbent articles and methods to make them are for example described in the following publications and literature cited therein and are expressly incorporated into the present invention: EP 2 301 499 A1, EP 2 314 264 A1, EP 2 387 981 A1, EP 2 486 901 A1, EP 2 524 679 A1, EP 2 524 679 A1, EP 2 524 680 A1, EP 2 565 031 A1, U.S. Pat. No. 6,972,011, US 2011/0162989, US 2011/0270204, WO 2010/004894 A1, WO 2010/004895 A1, WO 2010/076857 A1, WO 2010/082373 A1, WO 2010/118409 A1, WO 2010/133529 A2, WO 2010/143635 A1, WO 2011/084981 A1, WO 2011/086841 A1, WO 2011/086842 A1, WO 2011/086843 A1, WO 2011/086844 A1, WO 2011/117997 A1, WO 2011/136087 A1, WO 2012/048879 A1, WO 2012/052173 A1 and WO 2012/052172 A1, U.S. Pat. No. 7,378,568 B2.

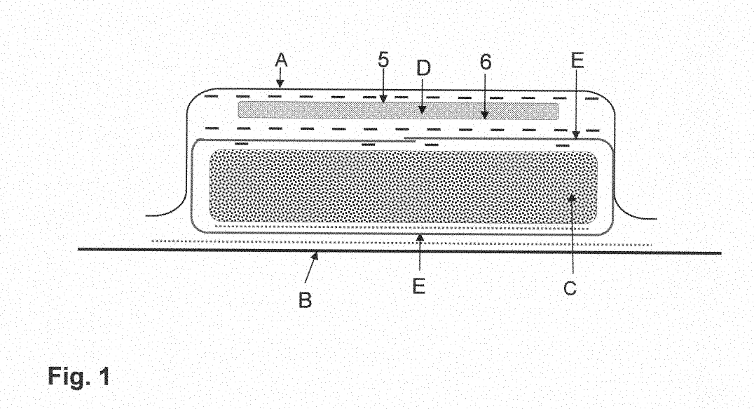

FIG. 1 shows a cross section of an example of an absorbent article according to the invention.

An absorbent article according to the invention preferably comprise as shown in FIG. 1 an upper liquid-pervious layer (A), a lower liquid-impervious layer (B) at least one fluid absorbent core (C) between (A) and (B); and an acquisition-distribution layer (D) between (A) and (C) having a three dimensional apertured structure having a femal side (5) and a male side (6), wherein the femal side presents a multiplicity of openings extending in the form of through holes in the direction of a lower surface of the same structure being the male side, an optional tissue layer disposed immediately above and/or below (C) or wrapped fully or partially around (C).

Liquid-Pervious Layer (A)

The liquid-pervious layer (A) is the layer which is in direct contact with the skin. Thus, the liquid-pervious layer is preferably compliant, soft feeling and non-irritating to the consumer's skin. Generally, the term “liquid-pervious” is understood thus permitting liquids, i.e. body fluids such as urine, menses and/or vaginal fluids to readily penetrate through its thickness. The principle function of the liquid-pervious layer is the acquisition and transport of body fluids from the wearer towards the fluid-absorbent core. Typically liquid-pervious layers are formed from any materials known in the art such as nonwoven material, films or combinations thereof. Suitable liquid-pervious layers (A) consist of customary synthetic or semisynthetic fibers or bicomponent fibers or films of polyester, polyolefins, rayon or natural fibers or any combinations thereof. In the case of nonwoven materials, the fibers should generally be bound by binders such as polyacrylates. Additionally the liquid-pervious layer may contain elastic compositions thus showing elastic characteristics allowing to be stretched in one or two directions.

Suitable synthetic fibers are made from polyvinyl chloride, polyvinyl fluoride, polytetrafluorethylene, polyvinylidene chloride, polyacrylics, polyvinyl acetate, polyethylvinyl acetate, non-soluble or soluble polyvinyl alcohol, polyolefins such as polyethylene, polypropylene, polyamides, polyesters, polyurethanes, polystyrenes and the like.

Examples for films are apertured formed thermoplastic films, apertured plastic films, hydro-formed thermoplastic films, reticulated thermoplastic films, porous foams, reticulated foams, and thermoplastic scrims.

Examples of suitable modified or unmodified natural fibers include cotton, bagasse, kemp, flax, silk, wool, wood pulp, chemically modified wood pulp, jute, rayon, ethyl cellulose, and cellulose acetate.

Suitable wood pulp fibers can be obtained by chemical processes such as the Kraft and sulfite processes, as well as from mechanical processes, such as ground wood, refiner mechanical, thermo-mechanical, chemi-mechanical and chemi-thermo-mechanical pulp processes. Further, recycled wood pulp fibers, bleached, unbleached, elementally chlorine free (ECF) or total chlorine free (TCF) wood pulp fibers can be used.

The fibrous material may comprise only natural fibers or synthetic fibers or any combination thereof. Preferred materials are polyester, rayon and blends thereof, polyethylene, and polypropylene.

The fibrous material as a component of the fluid-absorbent compositions may be hydrophilic, hydrophobic or can be a combination of both hydrophilic and hydrophobic fibers. The definition of hydrophilic is given in the section “definitions” in the chapter above. The selection of the ratio hydrophilic/hydrophobic and accordingly the amount of hydrophilic and hydrophobic fibers within fluid-absorbent composition will depend upon fluid handling properties and the amount of water-absorbent polymer particles of the resulting fluid-absorbent composition. Such, the use of hydrophobic fibers is preferred if the fluid-absorbent composition is adjacent to the wearer of the fluid-absorbent article, that is to be used to replace partially or completely the upper liquid-pervious layer, preferably formed from hydrophobic nonwoven materials. Hydrophobic fibers can also be member of the lower breathable, but fluid-impervious layer, acting there as a fluid-impervious barrier.

Examples for hydrophilic fibers are cellulosic fibers, modified cellulosic fibers, rayon, polyester fibers such as polyethylen terephthalate, hydrophilic nylon and the like. Hydrophilic fibers can also be obtained from hydrophobic fibers which are hydrophilized by e. g. surfactant-treating or silica-treating. Thus, hydrophilic thermoplastic fibers derived from polyolefins such as polypropylene, polyamides, polystyrenes or the like by surfactant-treating or silica-treating.

To increase the strength and the integrity of the upper-layer, the fibers should generally show bonding sites, which act as crosslinks between the fibers within the layer.

Technologies for consolidating fibers in a web are mechanical bonding, thermal bonding and chemical bonding. In the process of mechanical bonding the fibers are entangled mechanically, e.g., by water jets (spunlace) to give integrity to the web. Thermal bonding is carried out by means of raising the temperature in the presence of low-melting polymers. Examples for thermal bonding processes are spunbonding, through-air bonding and resin bonding.

Preferred means of increasing the integrity are thermal bonding, spunbonding, resin bonding, through-air bonding and/or spunlace.

In the case of thermal bonding, thermoplastic material is added to the fibers. Upon thermal treatment at least a portion of this thermoplastic material is melting and migrates to intersections of the fibers caused by capillary effects. These intersections solidify to bond sites after cooling and increase the integrity of the fibrous matrix. Moreover, in the case of chemically stiffened cellulosic fibers, melting and migration of the thermoplastic material has the effect of increasing the pore size of the resultant fibrous layer while maintaining its density and basis weight. Upon wetting, the structure and integrity of the layer remains stable. In summary, the addition of thermoplastic material leads to improved fluid permeability of discharged body fluids and thus to improved acquisition properties.

Suitable thermoplastic materials including polyolefins such as polyethylene and polypropylene, polyesters, copolyesters, polyvinyl acetate, polyethylvinyl acetate, polyvinyl chloride, polyvinylidene chloride, polyacrylics, polyamides, copolyamides, polystyrenes, polyurethanes and copolymers of any of the mentioned polymers.

Suitable thermoplastic fibers can be made from a single polymer that is a monocomponent fiber. Alternatively, they can be made from more than one polymer, e.g., bi-component or multicomponent fibers. The term “bicomponent fibers” refers to thermoplastic fibers that comprise a core fiber made from a different fiber material than the shell. Typically, both fiber materials have different melting points, wherein generally the sheath melts at lower temperatures. Bi-component fibers can be concentric or eccentric depending whether the sheath has a thickness that is even or uneven through the cross-sectional area of the bi-component fiber. Advantage is given for eccentric bi-component fibers showing a higher compressive strength at lower fiber thickness. Further bi-component fibers can show the feature “uncrimped” (unbent) or “crimped” (bent), further bi-component fibers can demonstrate differing aspects of surface lubricity.

Examples of bi-component fibers include the following polymer combinations: polyethylene/polypropylene, polyethylvinyl acetate/polypropylene, polyethylene/polyester, polypropylene/polyester, copolyester/polyester and the like.

Suitable thermoplastic materials have a melting point of lower temperatures that will damage the fibers of the layer; but not lower than temperatures, where usually the fluid-absorbent articles are stored. Preferably the melting point is between about 75° C. and 175° C. The typical length of thermoplastic fibers is from about 0.4 to 6 cm, preferably from about 0.5 to 1 cm. The diameter of thermoplastic fibers is defined in terms of either denier (grams per 9000 meters) or dtex (grams per 10 000 meters). Typical thermoplastic fibers have a dtex in the range from about 1.2 to 20, preferably from about 1.4 to 10.

A further mean of increasing the integrity of the fluid-absorbent composition is the spunbonding technology. The nature of the production of fibrous layers by means of spunbonding is based on the direct spinning of polymeric granulates into continuous filaments and subsequently manufacturing the fibrous layer.

Spunbond fabrics are produced by depositing extruded, spun fibers onto a moving belt in a uniform random manner followed by thermal bonding the fibers. The fibers are separated during the web laying process by air jets. Fiber bonds are generated by applying heated rolls or hot needles to partially melt the polymer and fuse the fibers together. Since molecular orientation increases the melting point, fibers that are not highly drawn can be used as thermal binding fibers. Polyethylene or random ethylene/propylene copolymers are used as low melting bonding sites.

Besides spunbonding, the technology of resin bonding also belongs to thermal bonding subjects. Using this technology to generate bonding sites, specific adhesives, based on e.g. epoxy, polyurethane and acrylic are added to the fibrous material and the resulting matrix is thermically treated. Thus the web is bonded with resin and/or thermal plastic resins dispersed within the fibrous material.

As a further thermal bonding technology through-air bonding involves the application of hot air to the surface of the fibrous fabric. The hot air is circulated just above the fibrous fabric, but does not push through the fibrous fabric. Bonding sites are generated by the addition of binders. Suitable binders used in through-air thermal bonding include crystalline binder fi-hers, bi-component binder fibers, and powders. When using crystalline binder fibers or powders, the binder melts entirely and forms molten droplets throughout the nonwoven's cross-section. Bonding occurs at these points upon cooling. In the case of sheath/core binder fibers, the sheath is the binder and the core is the carrier fiber. Products manufactured using through-air ovens tend to be bulky, open, soft, strong, extensible, breathable and absorbent. Through-air bonding followed by immediate cold calendering results in a thickness between a hot roll calendered product and one that has been though-air bonded without compression. Even after cold calendering, this product is softer, more flexible and more extensible than area-bond hot-calendered material.

Spunlacing (“hydroentanglement”) is a further method of increasing the integrity of a web. The formed web of loose fibers (usually air-laid or wet-laid) is first compacted and prewetted to eliminate air pockets. The technology of spunlacing uses multiple rows of fine high-speed jets of water to strike the web on a porous belt or moving perforated or patterned screen so that the fibers knot about one another. The water pressure generally increases from the first to the last injectors. Pressures as high as 150 bar are used to direct the water jets onto the web. This pressure is sufficient for most of the nonwoven fibers, although higher pressures are used in specialized applications.

The spunlace process is a nonwovens manufacturing system that employs jets of water to entangle fibers and thereby provide fabric integrity. Softness, drape, conformability, and relatively high strength are the major characteristics of spunlace nonwoven.

In newest researches benefits are found in some structural features of the resulting liquid-pervious layers.

Typically liquid-pervious layers (A) extend partially or wholly across the fluid-absorbent structure and can extend into and/or form part of all the preferred sideflaps, side wrapping elements, wings and ears.

Liquid-Impervious Layer (B)

The liquid-impervious layer (B) prevents the exudates absorbed and retained by the fluid-absorbent core from wetting articles which are in contact with the fluid-absorbent article, as for example bedsheets, pants, pyjamas and undergarments. The liquid-impervious layer (B) may thus comprise a woven or a nonwoven material, polymeric films such as thermoplastic film of polyethylene or polypropylene, or composite materials such as film-coated nonwoven material.

Suitable liquid-impervious layers include nonwoven, plastics and/or laminates of plastic and nonwoven. Both, the plastics and/or laminates of plastic and nonwoven may appropriately be breathable, that is, the liquid-impervious layer (B) can permit vapors to escape from the fluid-absorbent material. Thus the liquid-impervious layer has to have a definite water vapor transmission rate and at the same time the level of impermeability. To combine these features, suitable liquid-impervious layers including at least two layers, e.g. laminates from fibrous nonwoven having a specified basis weight and pore size, and a continuous three-dimensional film of e.g. polyvinylalcohol as the second layer having a specified thickness and optionally having pore structure. Such laminates acting as a barrier and showing no liquid transport or wet through. Thus, suitable liquid-impervious layers comprising at least a first breathable layer of a porous web which is a fibrous nonwoven, e.g. a composite web of a meltblown nonwoven layer or of a spunbonded nonwoven layer made from synthetic fibers and at least a second layer of a resilient three dimensional web consisting of a liquid-impervious polymeric film, e.g. plastics optionally having pores acting as capillaries, which are preferably not perpendicular to the plane of the film but are disposed at an angle of less than 90° relative to the plane of the film.

Suitable liquid-impervious layers are permeable for vapor. Preferably the liquid-impervious layer is constructed from vapor permeable material showing a water vapor transmission rate (WVTR) of at least about 100 gsm per 24 hours, preferably at least about 250 gsm per 24 hours and most preferred at least about 500 gsm per 24 hours.

Preferably the liquid-impervious layer (B) is made of nonwoven comprising hydrophobic materials, e.g. synthetic fibers or a liquid-impervious polymeric film comprising plastics e.g. polyethylene. The thickness of the liquid-impervious layer is preferably 15 to 30 μm.

Further, the liquid-impervious layer (B) is preferably made of a laminate of nonwoven and plastics comprising a nonwoven having a density of 12 to 15 gsm and a polyethylene layer having a thickness of about 10 to 20 μm.

The typically liquid-impervious layer (B) extends partially or wholly across the fluid-absorbent structure and can extend into and/or form part of all the preferred sideflaps, side wrapping elements, wings and ears.

Fluid-Absorbent Core (C)

The fluid-absorbent core (C) is disposed between the upper liquid-pervious layer (A) and the lower liquid-impervious layer (B). Suitable fluid-absorbent cores (C) may be selected from any of the fluid-absorbent core-systems known in the art provided that requirements such as vapor permeability, flexibility and thickness are met. Suitable fluid-absorbent cores refer to any fluid-absorbent composition whose primary function is to acquire, transport, distribute, absorb, store and retain discharged body fluids.

The top view area of the fluid-absorbent core (C) is preferably at least 200 cm², more preferably at least 250 cm², most preferably at least 300 cm². The top view area is the part of the core that is face-to-face to the upper liquid-pervious layer.

According to the present invention the fluid-absorbent core can include the following components:
1. an optional core cover
2. a fluid storage layer
3. an optional dusting layer

1. Optional Core Cover

In order to increase the integrity of the fluid-absorbent core, the core is provided with a cover. This cover may be at the top and/or at the bottom of the fluid-absorbent core with bonding at lateral juncture and/or bonding at the distal juncture by hot-melt, ultrasonic bonding, thermal bonding or combination of bonding techniques know to persons skilled in the art. Further, this cover may include the whole fluid-absorbent core with a unitary sheet of material and thus function as a wrap. Wrapping is possible as a full wrap, a partial wrap or as a C-Wrap.

The material of the core cover may comprise any known type of substrate, including webs, garments, textiles, films, tissues and laminates of two or more substrates or webs. The core cover material may comprise natural fibers, such as cellulose, cotton, flax, linen, hemp, wool, silk, fur, hair and naturally occurring mineral fibers. The core cover material may also comprise synthetic fibers such as rayon and lyocell (derived from cellulose), polysaccharides (starch), polyolefin fibers (polypropylene, polyethylene), polyamides, polyester, buta-diene-styrene block copolymers, polyurethane and combinations thereof. Preferably, the core cover comprises synthetic fibers or tissue.

The fibers may be mono- or multicomponent. Multicomponent fibers may comprise a homo-polymer, a copolymer or blends thereof.

2. Fluid-Storage Layer

The fluid-absorbent compositions included in the fluid-absorbent core comprise fibrous materials and water-absorbent polymer particles.

Fibers useful in the present invention include natural fibers and synthetic fibers. Examples of suitable modified or unmodified natural fibers are given in the chapter “Liquid-pervious Layer (A)” above. From those, wood pulp fibers are preferred.

Examples of suitable synthetic fibers are given in the chapter “Liquid-pervious Layer (A)” above. The fibrous material may comprise only natural fibers or synthetic fibers or any combination thereof.

The fibrous material as a component of the fluid-absorbent compositions may be hydrophilic, hydrophobic or can be a combination of both hydrophilic and hydrophobic fibers.

Generally for the use in a fluid-absorbent core, which is the embedded between the upper layer (A) and the lower layer (B), hydrophilic fibers are preferred. This is especially the case for fluid-absorbent compositions that are desired to quickly acquire, transfer and distribute discharged body fluids to other regions of the fluid-absorbent composition or fluid-absorbent core. The use of hydrophilic fibers is especially preferred for fluid-absorbent compositions comprising water-absorbent polymer particles.

Examples for hydrophilic fibers are given in the chapter “Liquid-pervious Layer (A)” above. Preferably, the fluid-absorbent core is made from viscose acetate, polyester and/or polypropylene.

The fibrous material of the fluid-absorbent core may be uniformly mixed to generate a homogenous or in-homogenous fluid-absorbent core. Alternatively the fibrous material may be concentrated or laid in separate layers optionally comprising water-absorbent polymer material. Suitable storage layers of the fluid-absorbent core comprising homogenous mixtures of fibrous materials comprising water-absorbent polymer material. Suitable storage layers of the fluid-absorbent core including a layered core-system comprise homogenous mixtures of fibrous materials and comprise water-absorbent polymer material, whereby each of the layers may be built from any fibrous material by means known in the art. The sequence of the layers may be directed such that a desired fluid acquisition, distribution and transfer results, depending on the amount and distribution of the inserted fluid-absorbent material, e.g. water-absorbent polymer particles. Preferably there are discrete zones of highest absorption rate or retention within the storage layer of the fluid-absorbent core, formed of layers or in-homogenous mixtures of the fibrous material, acting as a matrix for the incorporation of wa-ter-absorbent polymer particles. The zones may extend over the full area or may form only parts of the fluid-absorbent core.

Suitable fluid-absorbent cores comprise fibrous material and fluid-absorbent material. Suitable is any fluid-absorbent material that is capable of absorbing and retaining body fluids or body exudates such as cellulose wadding, modified and unmodified cellulose, crosslinked cellulose, laminates, composites, fluid-absorbent foams, materials described as in the chapter “Liquid-pervious Layer (A)” above, water-absorbent polymer particles and combinations thereof.

Typically the fluid-absorbent cores may contain a single type of water-absorbent polymer particles or may contain water-absorbent polymer particles derived from different kinds of water-absorbent polymer material. Thus, it is possible to add water-absorbent polymer particles from a single kind of polymer material or a mixture of water-absorbent polymer particles from different kinds of polymer materials, e.g. a mixture of regular water-absorbent polymer particles, derived from gel polymerization with water-absorbent polymer particles, derived from dropletization polymerization. Alternatively it is possible to add water-absorbent polymer particles derived from inverse suspension polymerization.

Alternatively it is possible to mix water-absorbent polymer particles showing different feature profiles. Thus, the fluid-absorbent core may contain water-absorbent polymer particles with uniform pH value, or it may contain water-absorbent polymer particles with different pH values, e.g. two- or more component mixtures from water-absorbent polymer particles with a pH in the range from about 4.0 to about 7.0. Preferably, applied mixtures deriving from mixtures of water-absorbent polymer particles got from gel polymerization or inverse suspension polymerization with a pH in the range from about 4.0 to about 7.0 and water-absorbent polymer particles got from drop polymerization.

Suitable fluid-absorbent cores are also manufactured from loose fibrous materials by adding water-absorbent particles and/or water-absorbent polymer fibers or mixtures thereof. The water-absorbent polymer fibers may be formed from a single type of water-absorbent polymer fiber or may contain water-absorbent polymer fibers from different polymeric materials. The addition of water-absorbent polymer fibers may be preferred for being distributed and incorporated easily into the fibrous structure and remaining better in place than water-absorbent polymer particles. Thus, the tendency of gel blocking caused by contacting each other is reduced. Further, water-absorbent polymer fibers are softer and more flexible.

In the process of manufacturing the fluid-absorbent core, water-absorbent polymer particles and/or fluid-absorbent fibers are brought together with structure forming compounds such as fibrous matrices. Thus, the water-absorbent polymer particles and/or fluid-absorbent fibers may be added during the process of forming the fluid-absorbent core from loose fibers. The fluid-absorbent core may be formed by mixing water-absorbent polymer particles and/or fluid-absorbent fibers with fibrous materials of the matrix at the same time or adding one component to the mixture of two or more other components either at the same time or by continuously adding.

Suitable fluid-absorbent cores including mixtures of water-absorbent polymer particles and/or fluid-absorbent fibers and fibrous material building matrices for the incorporation of the fluid-absorbent material. Such mixtures can be formed homogenously, that is all components are mixed together to get a homogenous structure. The amount of the fluid-absorbent materials may be uniform throughout the fluid-absorbent core, or may vary, e.g. between the central region and the distal region to give a profiled core concerning the concentration of fluid-absorbent material.

Techniques of application of the water-absorbent polymer materials into the absorbent core are known to persons skilled in the art and may be volumetric, loss-in-weight or gravimetric. Known techniques include the application by vibrating systems, single and multiple auger systems, dosing roll, weigh belt, fluid bed volumetric systems and gravitational sprinkle and/or spray systems. Further techniques of insertion are falling dosage systems consensus and contradictory pneumatic application or vacuum printing method of applying the fluid absorbent polymer materials.

Preferably drum-forming techniques are used where the fluid-absorbent core is formed in cavities of a drum rotating about a horizontal axis and being fed at a point on its periphery with a flow of water-absorbent polymer particles and/or fluid-absorbent fibers and fibrous material. The cylindrical surface of the drum on which the fluid-absorbent core is formed is surmounted by a hood, into which said flow is fed pneumatically from the top, bottom or tangentially. The inside of the hood may also contain the outlet of a feed duct, from which discrete quantities of additional water-absorbent polymer particles are dispensed by intermittently operating valve means under pressure. However, using prior art drum-forming techniques it is not possible to obtain uniform distribution of discrete quantities of water-absorbent polymer particles. Thus, in order to get profiled structures having different concentrations of water-absorbent polymer particles in discrete areas, it is preferred to use the technique written in WO 2010103453 in detail. Using this unit for the production of absorbent cores, by which a defined proportion of water-absorbent polymer particles are dispensed intermittently and controllably by adjustable elements controlling position and speed of application, it is possible to apply discrete quantities of water-absorbent polymer particles to a circumscribed area of precise geometrical shape.

Suitable fluid-absorbent cores may also include layers, which are formed by the process of manufacturing the fluid-absorbent article. The layered structure may be formed by subsequently generating the different layers in z-direction.

Alternatively a core-structure can be formed from two or more preformed layers to get a layered fluid-absorbent core. The layers may have different concentrations of water-absorbent polymer material showing concentrations in the range from about 10 to 95%. These uniform or different layers can be fixed to each other at their adjacent plane surfaces. Alternatively, the layers may be combined in a way that a plurality of chambers are formed, in which separately water-absorbent polymer material is incorporated.

Furthermore it can be preferred that the water-absorbent polymer particles are placed within the core in discrete regions even without chambers, e.g. supported by at least an adhesive.

Suitable preformed layers are processed as e.g. air-laid, wet-laid, laminate or composite structure.

Alternatively layers of other materials can be added, e.g. layers of opened or closed celled foams or perforated films. Included are also laminates of at least two layers comprising said water-absorbent polymer material.

Alternatively a core-structure can be formed from two or more layers, formed of e.g. nonwoven and/or thermoplastic materials containing water-absorbent polymer particles discretely contained in closed pockets. Such structures are preferably used for forming ultrathin absorbent products. The pockets are free of cellulose pulp. The bonds to define pockets are formed e.g. by intersection of ultrasonic contact areas between two thermoplastic containment layers. Further methods of immobilization of particulate fluid-absorbent material as well as the joining of layers in a layered structure are explained later on in more detail.

Alternatively a core-structure for ultrathin fluid-absorbent products can be formed from absorbent paper, e.g. a thin and flexible single layer of any suitable absorbent material known in the art including, but not limited to, short-fiber air-laid nonwoven materials; nonwoven of materials such as polyethylene, polypropylene, nylon, polyester, and the like; cellulosic fi-Brous materials such as paper tissue or towels known in the art, wax-coated papers, corrugated paper materials, and the like; or fluff pulp. The layer is macroscopically two-dimensional and planar and of very low thickness compared to the other dimensions. Said single layer may also incorporate superabsorbent material throughout the layer. Said single layer may further incorporate bi-component binding fibers. It may be also preferred to combine at least two of such layers in a core structure.

Water-absorbent polymer material may be incorporated as e.g. water-absorbent polymer fibers and/or water-absorbent polymer particles. Water-absorbent polymer particles may be bond to said single layer on one or both sides by attachment means known in the art.

Alternatively said absorbent paper may be formed from more layers, e.g. a layered absorbent sheet comprising a first layer on the wearer side, a second layer on the non-absorbing side and water-absorbent polymer particles in between or coated on one or both sides of the sheet layers.

The absorbent paper layer has a total basis weight ranging from about 100 gsm to about 2000 gsm, preferably from about 200 gsm to about 750 gsm, and more preferrably from about 400 gsm to about 600 gsm.

Further a composite structure can be formed from a carrier layer (e.g. a polymer film), onto which the water-absorbent polymer material is affixed. The fixation can be done at one side or at both sides. The carrier layer may be pervious or impervious for body-fluids.

Alternatively, it is possible to add monomer solution after the formation of a layer or onto a carrier layer and polymerize the coating solution by means of UV-induced polymerization technologies. Thus, “in situ”-polymerization is a further method for the application of water-absorbent polymers.

Thus, suitable fluid-absorbent cores comprising from 0 to 60% by weight a fibrous material and from 40 to 100% by weight water-absorbent polymer particles; preferably from 25 to 45% by weight a fibrous material and from 55 to 75% by weight water-absorbent polymer particles.

It is particularly preferred according to the present invention that the fluid-absorbent core of the inventive fluid-absorbent article comprises at least 40% by weight of water-absorbent polymer particles, more preferred at least 50% by weight of water-absorbent polymer particles, most preferred at least 70% by weight of water-absorbent polymer particles, particularly preferred at least 90% by weight of water-absorbent polymer particles.

It is preferred that the fluid-absorbent core comprises less than 50% by weight fibrous material, more preferred less than 45%, most preferred less than 10% by weight fibrous material.

According to the invention it is preferred that the fluid-absorbent core comprises not more than 10% by weight of an adhesive.

The quantity of water-absorbent polymer particles and/or fluid-absorbent fibers within the fluid-absorbent core is from 3 to 20 g, preferably from 4 to 18, more preferably from 6 to 16 g, and from 8 to 13 g in the case of maxi-diapers, and in the case of incontinence products up to about 50 g.

Typically fluid-absorbent articles comprising at least an upper liquid-pervious layer (A), at least a lower liquid-impervious layer (B) and at least one fluid-absorbent core between the layer (A) and the layer (B) besides other optional layers. In order to increase the control of body fluid absorption and/or to increase the flexibility in the ratio weight percentages of wa-ter-absorbent polymer particles to fibrous matrix it may be advantageous to add one or more further fluid-absorbent cores. The addition of a second fluid-absorbent core to the first fluid-absorbent core offers more possibilities in body fluid transfer and distribution. Moreover higher quantities of discharged body fluids can be retained. Having the opportunity of combining several layers showing different water-absorbent polymer concentration and content, it is possible to reduce the thickness of the fluid-absorbent article to a minimum even if there are several fluid-absorbent cores included.

Suitable fluid-absorbent cores may be formed from any material known in the art which is designed to acquire, transfer, and retain discharged body fluids. The technology of manufacturing may also be anyone known in the art. Preferred technologies include the application of monomer-solution to a transported fibrous matrix and thereby polymerizing, known as in-situ technology, or the manufacturing of air-laid composites.

Suitable fluid-absorbent articles are including single or multi-core systems in any combination with other layers which are typically found in fluid-absorbent articles. Preferred fluid-absorbent articles include single- or double-core systems; most preferably fluid-absorbent articles include a single fluid-absorbent core.

The fluid-absorbent core typically has a uniform size or profile. Suitable fluid-absorbent cores can also have profiled structures, concerning the shape of the core and/or the content of water-absorbent polymer particles and/or the distribution of the water-absorbent polymer particles and/or the dimensions of the different layers if a layered fluid-absorbent core is present.

It is known that absorbent cores providing a good wet immobilization by combining several layers, e.g. a substrate layer, layers of water-absorbent polymer and layers of thermoplastic material. Suitable absorbent cores may also comprise tissue or tissue laminates. Known in the art are single or double layer tissue laminates formed by folding the tissue or the tissue laminate onto itself.

These layers or foldings are preferably joined to each e.g. by addition of adhesives or by mechanical, thermal or ultrasonic bonding or combinations thereof. Water-absorbent polymer particles may be comprised within or between the individual layers, e.g. by forming separate water-absorbent polymer-layers.

Thus, according to the number of layers or the height of a voluminous core, the resulting thickness of the fluid-absorbent core will be determined. Thus, fluid-absorbent cores may be flat as one layer (plateau) or have three-dimensional profile.

Generally the upper liquid-pervious layer (A) and the lower liquid-impervious layer (B) may be shaped and sized according to the requirements of the various types of fluid-absorbent articles and to accommodate various wearer's sizes. Thus, the combination of the upper liquid-pervious layer and the lower liquid-impervious layer may have all dimensions or shapes known in the art. Suitable combinations have an hourglass shape, rectangular shape, trapezoidal shape, t- or double t-shape or showing anatomical dimensions.

The fluid-absorbent core may comprise additional additives typically present in fluid-absorbent articles known in the art. Exemplary additives are fibers for reinforcing and stabilizing the fluid-absorbent core. Preferably polyethylene is used for reinforcing the fluid-absorbent core.

Further suitable stabilizer for reinforcing the fluid-absorbent core are materials acting as binder.

In varying the kind of binder material or the amount of binder used in different regions of the fluid-absorbent core it is possible to get a profiled stabilization. For example, different binder materials exhibiting different melting temperatures may be used in regions of the fluid-absorbent core, e.g. the lower melting one in the central region of the core, and the higher melting in the distal regions. Suitable binder materials may be adhesive or non-adhesive fibers, continuously or discontinuously extruded fibers, bi-component staple fibers, non-elastomeric fibers and sprayed liquid binder or any combination of these binder materials.

Further, thermoplastic compositions usually are added to increase the integrity of the core layer. Thermoplastic compositions may comprise a single type of thermoplastic polymers or a blend of thermoplastic polymers. Alternatively, the thermoplastic composition may comprise hot melt adhesives comprising at least one thermoplastic polymer together with thermoplastic diluents such as tackifiers, plasticizers or other additives, e.g. antioxidants. The thermoplastic composition may further comprise pressure sensitive hot melt adhesives comprising e.g. crystalline polypropylene and an amorphous polyalphaolefin or styrene block copolymer and mixture of waxes.

Suitable thermoplastic polymers are styrenic block copolymers including A-B-A triblock segments, A-B diblock segments and (A-B)ₙ radial block copolymer segments. The letter A designs non-elastomeric polymer segments, e.g. polystyrene, and B stands for unsaturated conjugated diene or their (partly) hydrogenated form. Preferably B comprises isoprene, butadiene, ethylene/butylene (hydrogenated butadiene), ethylene/propylene (hydrogenated isoprene) and mixtures thereof.

Other suitable thermoplastic polymers are amorphous polyolefins, amorphous polyalphaolefins and metallocene polyolefins.

Concerning odor control, perfumes and/or odor control additives are optionally added. Suitable odor control additives are all substances of reducing odor developed in carrying fluid-absorbent articles over time known in the art. Thus, suitable odor control additives are inorganic materials, such as zeolites, activated carbon, bentonite, silica, aerosile, kieselguhr, clay; chelants such as ethylenediamine tetraacetic acid (EDTA), cyclodextrins, aminopolycarbonic acids, ethylenediamine tetramethylene phosphonic acid, aminophosphate, polyfunctional aromates, N,N-disuccinic acid.

Suitable odor control additives are further antimicrobial agents such as quaternary ammonium, phenolic, amide and nitro compounds and mixtures thereof; bactericides such as silver salts, zinc salts, cetylpyridinium chloride and/or triclosan as well as surfactants having an HLB value of less than 12.

Suitable odor control additives are further compounds with anhydride groups such as maleic-, itaconic-, polymaleic- or polyitaconic anhydride, copolymers of maleic acid with C₂-C₈ olefins or styrene, polymaleic anhydride or copolymers of maleic anhydride with isobutene, di-isobutene or styrene, compounds with acid groups such as ascorbic, benzoic, citric, salicylic or sorbic acid and fluid-soluble polymers of monomers with acid groups, homo- or copolymers of C₃-C₅ mono-unsaturated carboxylic acids.

Suitable odor control additives are further perfumes such as allyl caproate, allyl cyclohexaneacetate, allyl cyclohexanepropionate, allyl heptanoate, amyl acetate, amyl propionate, anethol, anixic aldehyde, anisole, benzaldehyde, benzyl acetete, benzyl acetone, benzyl alcohole, benzyl butyrate, benzyl formate, camphene, camphor gum, laevo-carveol, cinnamyl formate, cis-jasmone, citral, citronellol and its derivatives, cuminic alcohol and its derivatives, cyclal C, dimethyl benzyl carbinol and its derivatives, dimethyl octanol and its derivatives, eucalyptol, geranyl derivatives, lavandulyl acetete, ligustral, d-limonene, linalool, linalyl derivatives, menthone and its derivatives, myrcene and its derivatives, neral, nerol, p-cresol, p-cymene, orange terpenes, alpha-ponene, 4-terpineol, thymol etc.

Masking agents are also used as odor control additives. Masking agents are in solid wall material encapsulated perfumes. Preferably, the wall material comprises a fluid-soluble cellular matrix which is used for time-delay release of the perfume ingredient.

Further suitable odor control additives are transition metals such as Cu, Ag, Zn; enzymes such as urease-inhibitors, starch, pH buffering material, chitin, green tea plant extracts, ion exchange resin, carbonate, bicarbonate, phosphate, sulfate or mixtures thereof.

Preferred odor control additives are green tea plant extracts, silica, zeolite, carbon, starch, chelating agent, pH buffering material, chitin, kieselguhr, clay, ion exchange resin, carbonate, bicarbonate, phosphate, sulfate, masking agent or mixtures thereof. Suitable concentrations of odor control additives are from about 0.5 to about 300 gsm.

Newest developments propose the addition of wetness indication additives. Besides electrical monitoring the wetness in the fluid-absorbent article, wetness indication additives comprising a hot melt adhesive with a wetness indicator are known. The wetness indication additive changes the colour from yellow to a relatively dark and deep blue. This colour change is readily perceivable through the liquid-impervious outer material of the fluid-absorbent article. Existing wetness indication is also achieved via application of water soluble ink patterned on the backsheet which disappears when wet.

Suitable wetness indication additives comprising a mixture of sorbitan monooleate and polyethoxylated hydrogenated castor oil. Preferably, the amount of the wetness indication ad-ditive is in the range of about 0.0001 to 2% by weight related to the weight of the fluid-absorbent core.

The basis weight of the fluid-absorbent core is in the range of 600 to 1200 gsm. The density of the fluid-absorbent core is in the range of 0.1 to 0.25 g/cm³. The thickness of the fluid-absorbent core is in the case of diapers in the range of 1 to 5 mm, preferably 1.5 to 3 mm, in the case of incontinence products in the range of 3 to 15 mm.

3. Optional Dusting Layer

An optional component for inclusion into the absorbent core is a dusting layer adjacent to. The dusting layer is a fibrous layer and may be placed on the top and/or the bottom of the absorbent core. Typically, the dusting layer is underlying the storage layer. This underlying layer is referred to as a dusting layer, since it serves as carrier for deposited water-absorbent polymer particles during the manufacturing process of the fluid-absorbent core. If the water-absorbent polymer material is in the form of macrostructures, films or flakes, the insertion of a dusting layer is not necessary. In the case of water-absorbent polymer particles derived from dropletization polymerization, the particles have a smooth surface with no edges. Also in this case, the addition of a dusting layer to the fluid-absorbent core is not necessary. On the other side, as a great advantage the dusting layer provides some additional fluid-handling properties such as wicking performance and may offer reduced incidence of pin-holing and or pock marking of the liquid impervious layer (B).

Preferably, the dusting layer is a fibrous layer comprising fluff (cellulose fibers).

Acquisition-Distribution Layer (D)

The acquisition-distribution layer (D) is located between the upper layer (A) and the fluid-absorbent core (C) and is preferably constructed to efficiently acquire discharged body fluids and to transfer and distribute them to other regions of the fluid-absorbent composition or to other layers, where the body fluids are immobilized and stored. Thus, the upper layer transfers the discharged liquid to the acquisition-distribution layer (D) for distributing it to the fluid-absorbent core.

The acquisition-distribution layer according to the invention is preferably a three dimension-ally apertured structure. It may be fibrous material arranged as a web or be a film.

Preferred acquisition-distribution layers exhibit basis weights in the range from 10 to 50 gsm, most preferred in the range from 20 to 30 gsm, depending on the thickness of the web or film.

In case of a web the fibers may be hydrophilic, hydrophobic or can be a combination of both hydrophilic and hydrophobic. It may be derived from natural fibers, synthetic fibers or a combination of both. Known examples of synthetical fibers are found in the Chapter “Liquid-pervious Layer (A)” Further hydrophilic synthetical fibers are preferred. Hydrophilic synthetical fibers may be obtained by chemical modification of hydrophobic fibers. Preferably, hydrophilization is carried out by surfactant treatment of hydrophobic fibers. Thus the surface of the hydrophobic fiber can be rendered hydrophilic by treatment with a nonionic or ionic surfactant, e.g., by spraying the fiber with a surfactant or by dipping the fiber into a surfactant. Further preferred are permanent hydrophilic synthetic fibers.

The apertured film preferably manufactured from a liquid impervious thermoplastic material. The thermoplastic material of a typical apertured plastic film is selected from a group comprising polyethylene, polypropylene poly vinylchloride, starch base resins, polyvinylalcohol, polyurethanes, polycaprolactone and cellulose esters and combinations thereof.

The apertured film can also consist of other types of film that are not thermoplastic, e.g. hydro-formed film as e.g. described in U.S. Pat. No. 4,637,819 or U.S. Pat. No. 4,839,216.

The three-dimensional apertured structures having a femal side (5) and a male side (6), wherein the femal side presents a multiplicity of openings extending in the form of through holes in the direction of a lower surface of the same structure being the male side,

The apertures are defined by sidewalls in the structure extending from the female side and protrude from the male side of the structure.

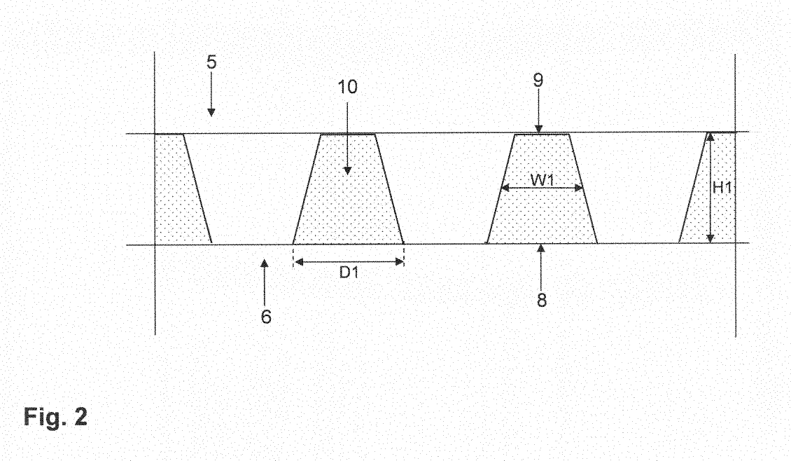

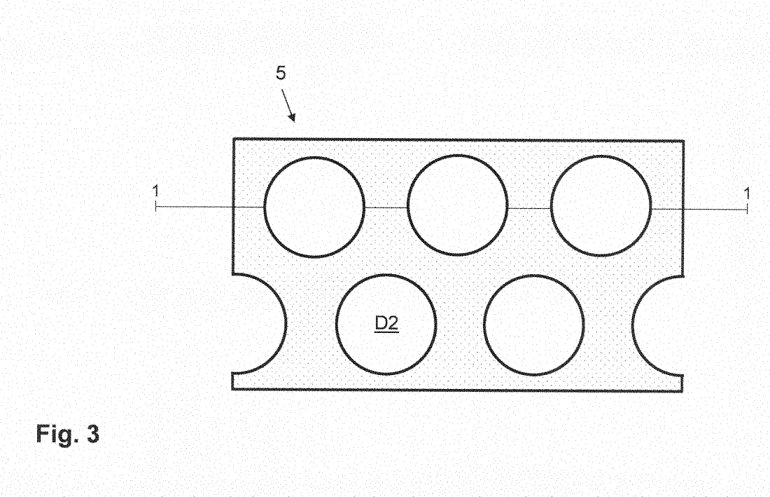

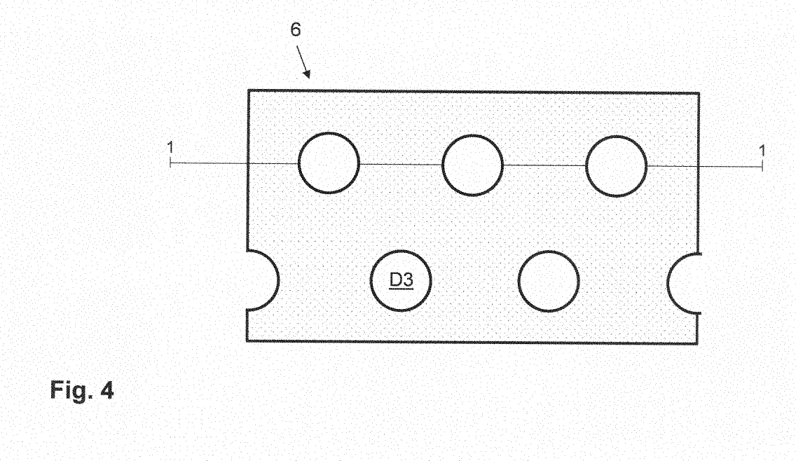

FIG. 2 shows cross section of a three dimensional aperture acquisition-distribution layer taken along line 1-1 of FIGS. 3 and 4

FIG. 3 Plan view of a female side of a three dimensional apertured acquisition-distribution layer

FIG. 4 Plan view of male side of a three dimensional apertured acquisition-distribution layer

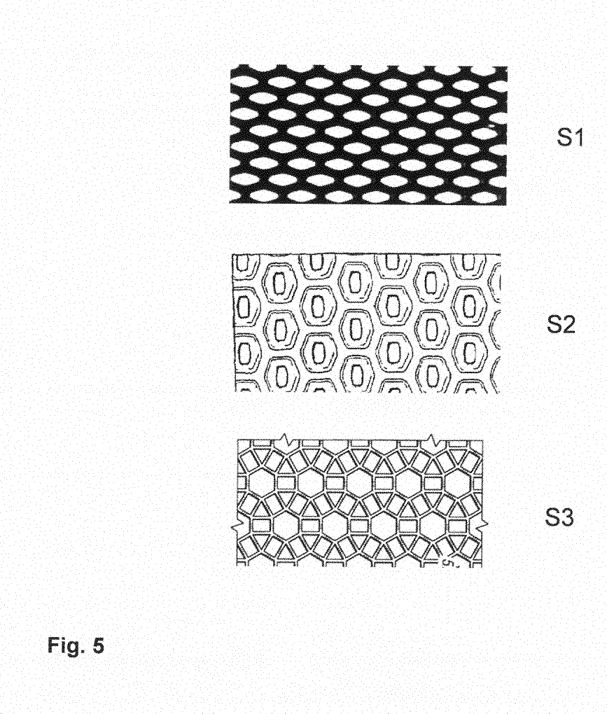

FIG. 5 Different shapes of the three dimensional openings in a three dimensional aperture acquisition-distribution layer

The reference numerals have the following meanings:
5 female side
6 male side
10 segment separating the holes
8 base of the segment
9 apex of the segment
D1 length D1 of the segment at the male side
D2 hole area on the female side
D3 hole area on the male side
W1 width of the segment at ½ H1
H1 height of the segment

According to one embodiment of the invention as especially shown in FIG. 2 mutually adjacent through holes of the acquisition-distribution layer (D) being separated by segments (10) of said acquisition-distribution layer (D) having a profile with symmetrical sides converging towards the female side. The profile of the segments preferably has a conic section as a cross section,

The acquisition-distribution layer provides a base (8) with a length D1 of the segment (10) at the male side and the apex (9) at the female side.

The height (H1) of the segments is given by the distance of the base (8) to the apex (9). The width of the segments (W1) is given by the width of the cross-section at H1/2 (this means H1×0.5).

The diameter and therefore the area of the through holes (apertures) at the female side (D2) is greater than the diameter, respectively the area, of the through holes at the male side (D3). This is illustrated using the example of circular apertures in FIG. 3 showing a plan view of the female side and in FIG. 4 showing a plan view of the male side.

According to the invention the sum of the areas D2 of the acquisition-distribution layer (D) is at least 45%, preferably at least 50%, of the total area of the acquisition-distribution layer (D). It is preferred that the ratio of the sum of the aperture areas over the total area of the acquisition-distribution layer is at least 1 for acquisition-distribution layers (D) placed “regular side”.

The acquisition-distribution layer preferred in the inventive absorbent article, shows a ratio of the width of the segment W1 to the height H1 of below 1, preferably below 0.9.

It is preferred that the width of the segment W1 is below 500 μm, preferably below 400 μm.

The openings or apertures in the web or film preferably imparted with a pattern. The pattern may be hexagonal, circular, oval, elliptical, polygonal or any other suitable patterns or combinations thereof. Examples for ellipse (S1), hexagonal (S2) and polygonal apertures (S3) are shown in FIG. 5.

According to one embodiment of the invention the female side faces the upper layer (A) and the male side the absorbent core (C). The acquisition-distribution layer here is placed so called “regular side”.

According to another embodiment of the invention it is preferred that the male side faces the upper layer (A) and the female side the absorbent core (C). The acquisition-distribution layer here is placed so called “flipside”.

This setting especially ensures a fast liquid acquisition (high liquid acquisition rate) of the absorbent articles.

According to another embodiment of the invention it is preferred that on top of the acquisition-distribution layer (D) between the upper layer (A) and the acquisition-distribution layer (D) a layer of curly fibres is placed. Curly fibres (cross-linked cellulose) is well known and disclosed as such in EP427316, U.S. Pat. No. 5,549,791, WO98/27262, U.S. Pat. No. 6,184,271, EP429112, and EP 427317. Due to the curly fibres the acquirement of discharged body fluids and the transfer and distribution of the fluid are improved. The curly fibres are hydrophilic. They bind discharged body fluids reversible. The discharged fluids are hold and distributed within the layer and not directly transferred to the acquisition-distribution layer and the absorbent core.

This lead to a very efficient fluid distribution in the absorbent article which results in very efficient utilization of the absorbent core in total.

The inventive fluid-absorbent articles comprising fluid-absorbent particles in combination with three-dimensional perforated acquisition-distribution layer placed male side towards the top sheet show improved rewet (less than 1 g after the 5th insult) and liquid acquisition time (shorter than 2 min for the 5th insult).

The absorbent article according to the invention generally allows the reduction of the amount of fluid-absorbent particles within the core by up to 30% compared to absorbent articles containing irregular shaped SAP.

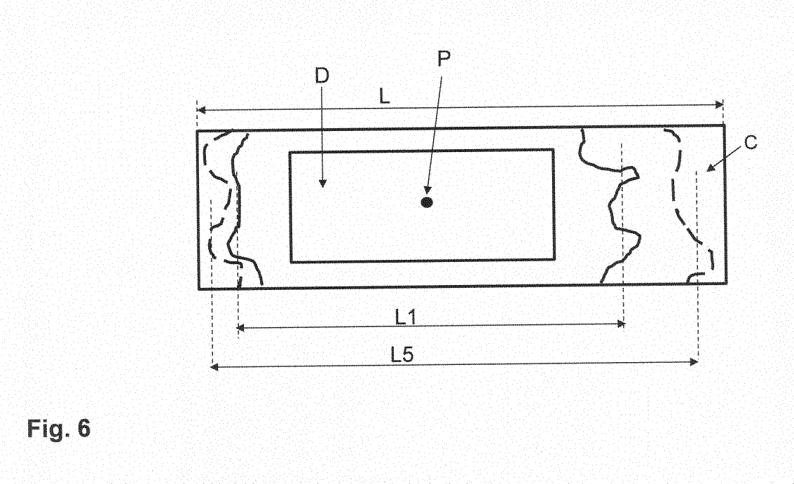

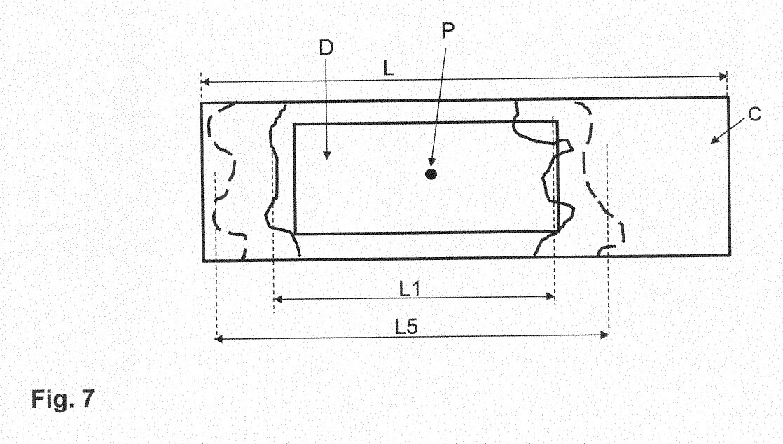

Furthermore absorbent articles according to the invention show, as illustrated in FIGS. 6 and 7 an improved liquid distribution.

FIG. 6 shows the liquid distribution in an absorbent article e.g. according to FIG. 1 comprising an air-through bonded fibrous acquisition distribution layer.

FIG. 7 illustrates the liquid distribution in an absorbent article according to the invention comprising a three dimensional apertured acquisition-distribution layer.

The reference numerals have the following meanings:
C absorbent core
D acquisition distribution layer
P insult point
L Length of the core
L1 average length of the insult zone after the first insult
L5 average length of the insult zone after the fifth insult

The length of the zone can be determined after each insult. After each insult according to the determination of the acquisition rate under load and Rewet Under Load (RUL) an insult zone (zone which absorb the fluid) is marked as shown schematically in FIGS. 6 and 7 for the 1^{st} and 5^{th} insult.

The length of the zone parallel to the largest extension of the absorbent article is determined. As illustrated in FIG. 7 the inventive absorbent article provides larger insult zones for each insult. After the fifth insult the liquid is almost equally distributed throughout the absorbent core. The amount of unused core area minimized.

This distribution further ensures the dryness of the user of the absorbent article even in gush situation.

The fibrous material of the acquisition-distribution layer may be fixed to increase the strength and the integrity of the layer. Technologies for consolidating fibers in a web are mechanical bonding, thermal bonding and chemical bonding. Detailed description of the different methods of increasing the integrity of the web is given in the Chapter “Liquid-pervious Layer (A)” above.

Optional Tissue Layer (E)

An optional tissue layer is disposed immediately above and/or below (C).

The material of the tissue layer may comprise any known type of substrate, including webs, garments, textiles and films. The tissue layer may comprise natural fibers, such as cellulose, cotton, flax, linen, hemp, wool, silk, fur, hair and naturally occurring mineral fibers. The tissue layer may also comprise synthetic fibers such as rayon and lyocell (derived from cellulose), polysaccharides (starch), polyolefin fibers (polypropylene, polyethylene), polyamides, polyester, butadiene-styrene block copolymers, polyurethane and combinations thereof. Preferably, the tissue layer comprises cellulose fibers.

Other Optional Components (F)

1. Leg Cuff

Typical leg cuffs comprising nonwoven materials which can be formed by direct extrusion processes during which the fibers and the nonwoven materials are formed at the same time, or by laying processes of preformed fibers which can be laid into nonwoven materials at a later point of time. Examples for direct extrusion processes include spunbonding, melt-blowing, solvent spinning, electrospinning and combinations thereof. Examples of laying processes include wet-laying and dry-laying (e.g. air-laying, carding) methods. Combinations of the processes above include spunbond-meltblown-spunbond (sms), spunbond-meltblow-meltblown-spunbond (smms), spunbond-carded (sc), spunbond-airlaid (sa), melt-blown-airlaid (ma) and combinations thereof. The combinations including direct extrusion can be combined at the same point in time or at a subsequent point in time. In the examples above, one or more individual layers can be produced by each process. Thus, “sms” means a three layer nonwoven material, “smsms” or “ssmms” means a five layer nonwoven material. Usually, small type letters (sms) designate individual layers, whereas capital letters (SMS) designate the compilation of similar adjacent layers.

Further, suitable leg cuffs are provided with elastic strands.

Preferred are leg cuffs from synthetic fibers showing the layer combinations sms, smms or smsms. Preferred are nonwovens with the density of 13 to 17 gsm. Preferably leg cuffs are provided with two elastic strands.

2. Elastics

The elastics are used for securely holding and flexibly closing the fluid-absorbent article around the wearers' body, e.g. the waist and the legs to improve containment and fit. Leg elastics are placed between the outer and inner layers or the fluid-absorbent article, or between the outer garment facing cover and the user facing bodyside liner. Suitable elastics comprising sheets, ribbons or strands of thermoplastic polyurethane, elastomeric materials, poly(ether-amide) block copolymers, thermoplastic rubbers, styrene-butadiene copolymers, silicon rubbers, natural rubbers, synthetic rubbers, styrene isoprene copolymers, styrene ethylene butylene copolymers, nylon copolymers, spandex fibers comprising segmented polyurethane and/or ethylene-vinyl acetate copolymer. The elastics may be secured to a substrate after being stretched, or secured to a stretched substrate. Otherwise, the elastics may be secured to a substrate and then elastisized or shrunk, e.g. by the application of heat.

3. Closing System

The closing system can include tape tabs, landing zone, elastomerics, pull ups and the belt system or combinations thereof

At least a part of the first waist region is attached to a part of the second waist region by the closing system to hold the fluid-absorbent article in place and to form leg openings and the waist of the fluid-absorbent article. Preferably the fluid-absorbent article is provided with a re-closable closing system.

The closing system is either re-sealable or permanent, including any material suitable for such a use, e.g. plastics, elastics, films, foams, nonwoven substrates, woven substrates, paper, tissue, laminates, fiber reinforced plastics and the like, or combinations thereof. Preferably the closing system includes flexible materials and works smooth and softly without irritating the wearer's skin.

One part of the closing elements is an adhesive tape, or comprises a pair of laterally extending tabs disposed on the lateral edges of the first waist region. Tape tabs are typically attached to the front body panel and extend laterally from each corner of the first waistband. These tape tabs include an adhesive inwardly facing surface which is typically protected prior to use by a thin, removable cover sheet.

Suitable tape tabs may be formed of thermoplastic polymers such as polyethylene, polyurethane, polystyrene, polycarbonate, polyester, ethylene vinyl acetate, ethylene vinyl alcohol, ethylene vinyl acetate acrylate or ethylene acrylic acid copolymers.

Suitable closing systems comprise further a hook portion of a hook and loop fastener and the target devices comprise the loop portion of a hook and loop fastener.

Suitable mechanical closing systems including a landing zone. Mechanical closing systems may fasten directly into the outer cover. The landing zone may act as an area of the fluid-absorbent article into which it is desirable to engage the tape tabs. The landing zone may include a base material and a plurality of tape tabs. The tape tabs may be embedded in the base material of the landing zone. The base material may include a loop material. The loop material may include a backing material and a layer of a nonwoven spunbond web attached to the backing material.

Thus suitable landing zones can be made by spunbonding. Spunbonded nonwoven are made from melt-spun fibers formed by extruding molten thermoplastic material. Preferred is bi-oriented polypropylene (BOPP), or brushed/closed loop in the case of mechanical closing systems.

Further, suitable mechanical closing systems including elasticomeric units serving as a flexible abdominal and/or dorsal discrete waist band, flexible abdomen and/or dorsal zones located at distal edge for fluid-absorbents articles, such as pants or pull-ups. The elasticomeric units enable the fluid-absorbent article to be pulled down by the wearer as e.g. a training pant.

Suitable pants-shaped fluid-absorbent article has front abdominal section, rear dorsal section, crotch section, side sections for connecting the front and rear sections in lateral direction, hip section, elastic waist region and liquid-tight outer layer. The hip section is arranged around the waist of the user. The disposable pants-shaped fluid-absorbent article (pull-up) has favorable flexibility, stretchability, leak-proof property and fit property, hence imparts excellent comfort to the wearer and offers improved mobility and discretion.

Suitable pull-ups comprising thermoplastic films, sheets and laminates having a low modulus, good tear strength and high elastic recovery.

Suitable closing systems may further comprise elastomerics for the production of elastic areas within the fastening devices of the fluid-absorbent article. Elastomerics provide a conformable fit of the fluid-absorbent article to the wearer at the waist and leg openings, while maintaining adequate performance against leakage.

Suitable elastomerics are elastomeric polymers or elastic adhesive materials showing vapor permeability and liquid barrier properties. Preferred elastomerics are retractable after elongation to a length equivalent to its original length.

Suitable closing systems further comprise a belt system, comprising waist-belt and leg-belts for flexibly securing the fluid-absorbent article on the body of the wearer and to provide an improved fit on the wearer. Suitable waist-belts comprising two elastic belts, a left elastic belt, and a right elastic belt. The left elastic belt is associated with each of the left angular edges. The right elastic belt associated with each of the right angular edges. The left and right side belts are elastically extended when the absorbent garment is laid flat. Each belt is connected to and extends between the front and rear of the fluid-absorbent article to form a waist hole and leg holes.

Preferably the belt system is made of elastomerics, thus providing a conformable fit of the fluid-absorbent article and maintaining adequate performance against leakage.

Preferred closing systems are so-called “elastic ears” attached with one side of the ear to the longitudinal side edges located at the rear dorsal longitudinal edge of the chassis of the fluid-absorbent article. Commercially available fluid-absorbent articles include stretchable ears or side panels which are made from a stretchable laminate e.g. nonwoven webs made of mono- or bi-component fibers. Especially preferred closing systems are stretchable laminates comprising a core of several layers each of different fibrous materials, e.g. meltblown fibers, spunbond fibers, containing multicomponent fibers having a core comprising a first polymer having a first melt temperature and a sheath comprising a second polymer having a second melt temperature; and a web of an elastomeric material as top and bottom surfaces to form said laminate.

### D. Fluid-Absorbent Article Construction

The present invention further relates to the joining of the components and layers, films, sheets, tissues or substrates mentioned above to provide the fluid-absorbent article. At least two, preferably all layers, films, sheets, tissues or substrates are joined.

Suitable fluid-absorbent articles include a single- or multiple fluid-absorbent core-system. Preferably fluid-absorbent articles include a single- or double fluid-absorbent core-system.

Suitable fluid-storage layers of the fluid-absorbent core comprising homogenous or in-homogenous mixtures of fibrous materials comprising water-absorbent polymer particles homogenously or in-homogenously dispersed in it. Suitable fluid-storage layers of the fluid-absorbent core including a layered fluid-absorbent core-system comprising homogenous mixtures of fibrous materials and optionally comprising water-absorbent polymer particles, whereby each of the layers may be prepared from any fibrous material by means known in the art.

In order to immobilize the water-absorbent polymer particles, the adjacent layers are fixed by the means of thermoplastic materials, thereby building connections throughout the whole surface or alternatively in discrete areas of junction. For the latter case, cavities or pockets are built carrying the water-absorbent particles. The areas of junction may have a regular or irregular pattern, e.g. aligned with the longitudinal axis of the fluid-absorbent core or in a pattern of polygons, e.g. pentagons or hexagons. The areas of junction itself may be of rectangular, circular or squared shape with diameters between about 0.5 mm and 2 mm. Fluid-absorbent articles comprising areas of junction show a better wet strength.

The construction of the products chassis and the components contained therein is made and controlled by the discrete application of hotmelt adhesives as known to people skilled in the art. Examples would be e.g. Dispomelt 505B, Dispomelt Cool 1101, as well as other specific function adhesives manufactured by National Starch, Henkel or Fuller. In order to ensure wicking of applied body fluids, preferred fluid-absorbent article show channels for better transport. Channels are formed by compressional forces of e.g. the top sheet against the fluid-absorbent core. Compressive forces may be applied e.g. by heat-treatment between two heated calendar rollers. As an effect of compression both on top sheet and fluid-absorbent core deform such that a channel is created. Body fluids are flowing along this channel to places where they are absorbed and leakage is prevented. Otherwise, compression leads to higher density; this is the second effect of the channel to canalize insulted fluids. Additionally, compressive forces on diaper construction improve the structural integrity of the fluid-absorbent article.

A possible embodiment of the present invention is shown in FIG. 1.

Thus, a fluid-absorbent article according to the invention comprising
(A) an upper liquid-pervious layer comprising a spunbond layer (coverstock)
(B) a lower liquid-impervious layer comprising a composite of a breathable polyethylene film
(C) a single fluid-absorbent core (C) between (A) and (B) comprising a homogeneous mixture of water-absorbent polymer particles and cellulose fibres
(D) an acquisition-distribution layer (D) between (A) and (C) having a size smaller than the fluid absorbent core (three dimensional film or air-through bonded nonwoven); the acquisition-distribution layer (D) has a female side (5) and a male side (6).

The construction of the products chassis and the components contained therein is made and controlled by the discrete application of hotmelt adhesives as attachment means as known to people skilled in the art. Examples would be e.g. Dispomelt 505B, Dispomelt Cool 1101, as well as other specific function adhesives manufactured by for example Bostik, Henkel or Fuller.

Methods:

The measurements should, unless stated otherwise, be carried out at an ambient temperature of 23±2° C. and a relative atmospheric humidity of 50±10%. The water-absorbent polymers are mixed thoroughly before the measurement.

The “WSP” standard test methods are described in: “Standard Test Methods for the Nonwovens Industry”, jointly issued by the “Worldwide Strategic Partners” EDANA (European Disposables and Nonwovens Association, Avenue Eugene Plasky, 157, 1030 Brussets, Belgium, www.edana.org) and INDA (Association of the Nonwoven Fabrics Industry, 1100 Crescent Green, Suite 115, Cary, N.C. 27518, U.S.A., www.inda.org). This publication is available both from EDANA and INDA.

Vortex

50.0±1.0 ml of 0.9% NaCl solution are added into a 100 ml beaker. A cylindrical stirrer bar (30×6 mm) is added and the saline solution is stirred on a stir plate at 60 rpm. 2.000±0.010 g of water-absorbent polymer particles are added to the beaker as quickly as possible, starting a stop watch as addition begins. The stopwatch is stopped when the surface of the mixture becomes “still” that means the surface has no turbulence, and while the mixture may still turn, the entire surface of particles turns as a unit. The displayed time of the stopwatch is recorded as Vortex time.

Acquisition Rate Under Load and Rewet Under Load (RUL)

The combined acquisition rate under load is the determination of the time needed for the diaper to completely absorb a certain amount of synthetic urine to ensure dryness of the diaper even in gush situations and the rewet under load test is the determination of the dryness of a diaper under a certain load. For testing a fluid-absorbent article, in this case a diaper, the diaper is insulted several times with defined amounts of synthetic urine under a load. Synthetic urine consists of a 9 g/I solution of sodium chloride in deionised water with a surface tension of (70±2) mN/m. The rewet under load is measured by the amount of fluid the article releases after being maintained at a pressure of 0.7 psi (49.2 g/cm²) for 10 min after commencement of each insult.

The fluid-absorbent article is clamped nonwoven side upward onto the inspection table. The insult point is marked accordingly with regard to the type and gender of the diaper to be tested (i.e. in the centre of the core for girl, 2.5 cm towards the front for unisex and 5 cm towards the front for boy). A 3.64 kg circular weight (10 cm diameter) having a central opening (2.3 cm diameter) with perspex tube is placed with on the previously marked insult point.

For the primary insult 100 g of aqueous saline solution (0.9% by weight) is poured into the perspex tube in one shot. Amount of time (A or acquisition time) needed for the fluid to be fully absorbed into the fluid-absorbent article is recorded. After 10 minutes have elapsed, the load is removed and the stack of 10 filter papers (Whatman®) having 9 cm diameter and known dry weight (w1) is placed over the insult point on the fluid-absorbent article. On top of the filter paper, the 2.5 kg weight with 8 cm diameter is added. After 2 minutes have elapsed the weight is removed and filter paper reweighed giving the wet weight value (w2).

The rewet under load is calculated as follows:

RUL[g]=w2−w1

For the rewet under load of the secondary insult the procedure for the primary insult is repeated. 50 g of aqueous saline solution (0.9% by weight) and 20 filter papers are used.

For the rewet under load of the tertiary and following insults the procedure for the primary insult is repeated. For each of the following insults 3^{rd}, 4^{th} and 5^{th} 50 g of aqueous saline solution (0.9% by weight) and 30, 40 and 50 filter papers respectively are used.

For each insult the corresponding acquisition time (A1, A2, A3, A4, A5) is recorded.

The acquisition rate is calculated as follows:

ACQ[g/sec]=liquid per insult[g]/A(acquisition time)[sec]

Determination of the Length of the Insult Zone

After each insult according to the determination of the acquisition rate under load and Rewet Under Load (RUL) the insult zone (zone which absorb the fluid) is marked as shown schematically in FIGS. 6 and 7 for the 1^{st} and 5^{th} insult.

Then the length (L1, L2, L3, L4, L5 per each insult) of the area parallel to the largest extension of the absorbent article is determined.

Basis Weight

The basis weight is determined at discrete regions of the fluid-absorbent core: the front overall average; the insult zone and the back overall average.

The article nonwoven face is pinned upwards onto the inspection table. Then an insult point is marked on the fluid-absorbent article. The insult point is marked on the article accordingly with regard to the type and gender of the diaper to be tested (i.e. in the centre of the fluid-absorbent core for girl, 2.5 cm towards the front for unisex and 5 cm towards the front for boy).

Then lines for the following zones are marked on the fluid-absorbent article in dependence of the diaper to be tested, e. g. for boy diapers:
for the front overall average zone 5.5 cm forward of the center of the core to the front distal edge of the core;
for the insult zone 5.5 cm forward and 0.5 cm backwards of the center of the core;
for the back overall average zone 0.5 cm backward of the center of the core to the rear distal edge of the core

The length (ZL) and width (ZW) of each zone is recorded. Then the previously marked zones are cut out and the record weight (ZWT) of each zone is taken.

Before calculating the basis weight, the area of each zone must first be calculated as follows:

Zonal Area (ZA)=(ZW×ZL)[cm²]

The Zonal Basis Weight (ZBW) is then calculated as follows:

Zonal Basis Weight (ZBW)=ZWT/(ZW*ZL)*10000 [g/cm²]

For example, if ZW is 6 cm, ZL is 10 cm and ZWT is 4.5 g the Zonal Basis Weight (ZBW) is:

ZBW=4.5 g/(6 cm×10 cm)*10000=750 gsm

Conversion of Gram per Square Centimeter g/cm² to Gram per Square Meter g/m²:

10 000×g/cm²=g/m²

Conversion of Gram per Square Meter g/m² to Gram per Square Centimeter g/cm²:

0.0001×g/m²=g/cm²

Density of a Fluid-Absorbent Core

This test determines the density of the fluid-absorbent core in the point of interest.

The fluid-absorbent article is clamped nonwoven side up onto the inspection table. The insult point is marked on the article accordingly with regard to the type and gender of the diaper to be tested (i.e. in the centre of the fluid-absorbent core for girl, 2.5 cm towards the front for unisex and 5 cm towards the front for boy).

Next, a 6 cm×core width section is marked on the fluid-absorbent core with the point of interest in the centre of the section. Three readings of thickness of the section are taken using a Portable Thickness Gauge Model J100 (SDL Atlas, Inc.; Stockport; UK) and the average is recorded (T). The section of the fluid-absorbent core is cut out of and the weight of the cut out section is recorded (WT).

The density of the fluid-absorbent core is calculated as follows:

Density [g/cm³]=WT/(36 cm²×T)

Saline Flow Conductivity (SFC)

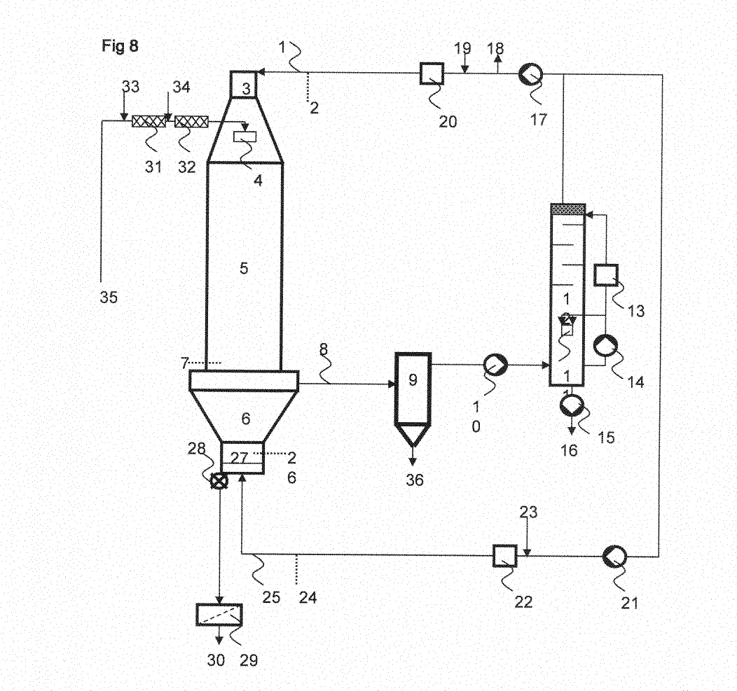

The saline flow conductivity is, as described in EP 0 640 330 A1, determined as the gel layer permeability of a swollen gel layer of water-absorbent polymer particles, although the apparatus described on page 19 and in FIG. 8 in the aforementioned patent application was modified to the effect that the glass frit (40) is no longer used, the plunger (39) consists of the same polymer material as the cylinder (37) and now comprises 21 bores having a diameter of 9.65 mm each distributed uniformly over the entire contact surface. The procedure and the evaluation of the measurement remains unchanged from EP 0 640 330 A1. The flow rate is recorded automatically.

The saline flow conductivity (SFC) is calculated as follows:

SFC [cm³s/g]=(Fg(t=0)×L0)/(d×A×WP),

where Fg(t=0) is the flow rate of NaCl solution in g/s, which is obtained by means of a linear regression analysis of the Fg(t) data of the flow determinations by extrapolation to t=0, L0 is the thickness of the gel layer in cm, d is the density of the NaCl solution in g/cm³, A is the surface area of the gel layer in cm² and WP is the hydrostatic pressure over the gel layer in dyn/cm².

Free Swell Rate (FSR)

1.00 g (=w1) of the dry water-absorbent polymer particles is weighed into a 25 ml glass beaker and is uniformly distributed on the base of the glass beaker. 20 ml of a 0.9% by weight sodium chloride solution are then dispensed into a second glass beaker, the content of this beaker is rapidly added to the first beaker and a stopwatch is started. As soon as the last drop of salt solution is absorbed, confirmed by the disappearance of the reflection on the liquid surface, the stopwatch is stopped. The exact amount of liquid poured from the second beaker and absorbed by the polymer in the first beaker is accurately determined by weighing back the second beaker (=w2). The time needed for the absorption, which was measured with the stopwatch, is denoted t. The disappearance of the last drop of liquid on the surface is defined as time t.

The free swell rate (FSR) is calculated as follows:

FSR [gigs]=w2/(w1×t)

When the moisture content of the hydrogel-forming polymer is more than 3% by weight, however, the weight w1 must be corrected for this moisture content.

Water Vapor Transmission Rate (WVTR) The water vapor transmission rate (WVTR) is determined according to the test method written in U.S. Pat. No. 6,217,890, column 32, lines 15 to 56.

Anticaking

The anticaking is determined according to the test method written in WO 2005/097881 A1, page 19, lines 14 to 24. For quantitative ranking grades are given between 1 and 5, whereby grade 1 does not leave any residue in the beaker and at grade 5 no material can be poured out of the beaker.

Residual Monomers

The level of residual monomers in the water-absorbent polymer particles is determined by the EDANA recommended test method No. WSP 210.3-(11) “Residual Monomers”.

Particle Size Distribution

The particle size distribution of the water-absorbent polymer particles is determined with the Camziser® image analysis system (Retsch Technology GmbH; Haan; Germany).

For determination of the average particle diameter and the particle diameter distribution the proportions of the particle fractions by volume are plotted in cumulated form and the average particle diameter is determined graphically.

The average particle diameter (APD) here is the value of the mesh size which gives rise to a cumulative 50% by weight.

The particle diameter distribution (PDD) is calculated as follows:

$$PDD = \frac{{x}_{2} - {x}_{1}}{APD} ,$$

wherein x₁ is the value of the mesh size which gives rise to a cumulative 90% by weight and x₂ is the value of the mesh size which gives rise to a cumulative 10% by weight.

Mean Sphericity

The mean sphericity is determined with the Camziser® image analysis system (Retsch Technology GmbH; Haan; Germany) using the particle diameter fraction from 100 to 1,000 μm.

Moisture Content

The moisture content of the water-absorbent polymer particles is determined by the EDANA recommended test method No. WSP 230.3 (11) “Mass Loss Upon Heating”.

Centrifuge Retention Capacity (CRC)

The centrifuge retention capacity of the water-absorbent polymer particles is determined by the EDANA recommended test method No. WSP 241.3 (11) “Free Swell Capacity in Saline, After Centrifugation”, wherein for higher values of the centrifuge retention capacity larger tea bags have to be used.

Absorbency Under No Load (AUNL)

The absorbency under no load of the water-absorbent polymer particles is determined analogously to the EDANA recommended test method No. WSP 242.3 (11) “Gravimetric Determination of Absorption Under Pressure”, except using a weight of 0.0 g/cm² instead of a weight of 21.0 g/cm².

Absorbency Under Load (AUL)

The absorbency under load of the water-absorbent polymer particles is determined by the EDANA recommended test method No. WSP 242.3 (11) “Gravimetric Determination of Absorption Under Pressure”

Absorbency Under High Load (AUHL)

The absorbency under high load of the water-absorbent polymer particles is determined analogously to the EDANA recommended test method No. WSP 242.3 (11) “Gravimetric Determination of Absorption Under Pressure”, except using a weight of 49.2 g/cm² instead of a weight of 21.0 g/cm².

Volumetric Absorbency Under Load (VAUL)

The volumetric absorbency under a load is used in order to measure the swelling kinetics, i.e. the characteristic swelling time, of water-absorbent polymer particles under different applied pressures. The height of swelling is recorded as a function of time.

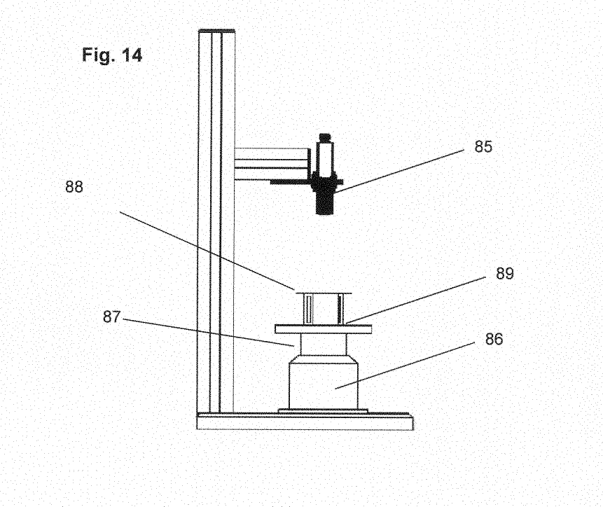

The set up is show in FIG. 14 and consists of
An ultrasonic distance sensor (85) type BUS M18K0-XBFX-030-S04K (Balluff GmbH, Neuhausen a.d.F.; Germany) is placed above the cell. The sensor receives ultrasound reflected by the metal plate. The sensor is connected to an electronic recorder.
A PTFE cell (86) having a diameter of 75 mm, a height of 73 mm and an internal diameter of 52 mm
A cylinder (87) made of metal or plastic having a diameter of 50 mm, a height of 71 mm and a mesh bottom)
A metal reflector (88) having a diameter of 57 mm and a height of 45 mm
Metal ring weights (89) having a diameter of 100 mm and weights calibrated to 278.0 g or 554.0 g

It is possible to adjust the pressure applied to the sample by changing the combination of cylinder (86) and metal ring (88) weight as summarized in the following tables:

| Available Equipment | Weight | psi |
|---|---|---|
| Metal reflector | 13.0 g | 0.009 |
| Plastic cylinder | 28.0 g | 0.020 |
| Metal cylinder | 126.0 g | 0.091 |
| Small ring weight | 278.0 g | 0.201 |
| Large ring weight | 554.0 g | 0.401 |

| Possible Combinations | psi |
|---|---|
| Metal reflector + plastic cylinder | 0.03 |
| Metal reflector + metal cylinder | 0.10 |
| Metal reflector + metal cylinder + small ring weight | 0.30 |
| Metal reflector + metal cylinder + large ring weight | 0.50 |
| Metal reflector + metal cylinder + small ring weight + | 0.70 |
| large ring weight | |

A sample of 2.0 g of water-absorbent polymer particles is placed in the PTFE cell (86). The cylinder (equipped with mesh bottom) and the metal reflector (88) on top of it are placed into the PTFE cell (86). In order to apply higher pressure, metal rings weights (89) can be placed on the cylinder.

60.0 g of aqueous saline solution (0.9% by weight) are added into the PTFE cell (86) with a syringe and the recording is started. During the swelling, the water-absorbent polymer particles push the cylinder (87) up and the changes in the distance between the metal reflector (88) and the sensor (85) are recorded.

After 120 minutes, the experiment is stopped and the recorded data are transferred from the recorder to a PC using a USB stick. The characteristic swelling time is calculated according to the equation Q(t)=Qₘₐₓ·(1−e^{-t/τ}) as described by “Modern Superabsorbent Polymer Technology” (page 155, equation 4.13) wherein Q(t) is the swelling of the superabsorbent which is monitored during the experiment, Qₘₐₓ corresponds to the maximum swelling reached after 120 minutes (end of the experiment) and τ is the characteristic swelling time (τ is the inverse rate constant k).

Using the add-in functionality “Solver” of Microsoft Excel software, a theoretical curve can be fitted to the measured data and the characteristic time for 0.03 psi is calculated.

The measurements are repeated for different pressures (0.1 psi, 0.3 psi, 0.5 psi and 0.7 psi) using combinations of cylinder and ring weights. The characteristic swelling times for the different pressures can be calculated using the equation Q(t)=Qₘₐₓ·(1−e^{-t/τ})

Wicking Absorption

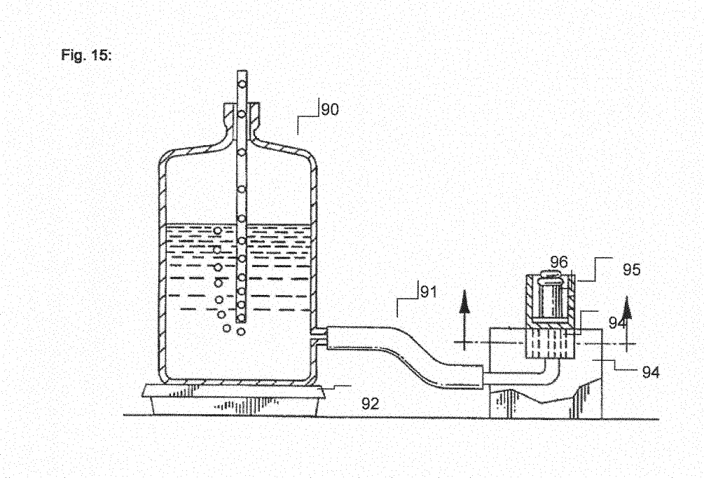

The wicking absorption is used in order to measure the total liquid uptake of water-absorbent polymer particles under applied pressure. The set-up is show in FIG. 15.

A 500 ml glass bottle (90) (scale division 100 mL, height 26.5 cm) equipped with an exit tube of Duran® glass, is filled with 500 mL of aqueous saline solution (0.9% by weight). The bottle has an opening at the bottom end which can be connected to the Plexiglas plate through a flexible hose (91).

A balance (92) connected to a computer is placed on Plexiglas block (area 20×26 cm², height 6 cm). The glass bottle is then placed on the balance.

A Plexiglas plate (93) (area: 11×11 cm², height: 3.5 cm) is placed on a lifting platform. A porosity P1 glass frit of 7 cm in diameter and 0.45 cm high (94) has been liquid-tightly embedded in the Plexiglas plate, i.e. the fluid exits through the pores of the frit and not via the edge between Plexiglas plate and frit. A Plexiglas tube leads through the outer shell of Plexiglas plate into the center of the Plexiglas plate up to the frit to ensure fluid transportation. The fluid tube is then connected with the flexible hose (35 cm in length, 1.0 cm external diameter, 0.7 cm internal diameter) to the glass bottle (90).

The lifting platform is used to adjust the upper side of the frit to the level of the bottom end of the glass bottle, so that an always atmospheric flux of fluid from the bottle to the measuring apparatus is ensured during measurement. The upper side of the frit is adjusted such that its surface is moist but there is no supernatant film of water on the frit.

The fluid in the glass bottle (90) is made up to 500 mL before every run.

In a Plexiglas cylinder (95) (7 cm in external diameter, 6 cm in internal diameter, 16 cm in height) and equipped with a 400 mesh (36 μm) at the bottom are placed 26 g of water-absorbent polymer particles. The surface of the water-absorbent polymer particles is smoothed. The fill level is about 1.5 cm. Then a weight (96) of 0.3 psi (21.0 g/cm²) is placed on top of the water-absorbent polymer particles.

The Plexiglas cylinder is placed on the (moist) frit and the electronic data recording started. A decrease in the weight of the balance is registered as a function of time. This then indicates how much aqueous saline solution has been absorbed by the swelling gel of water-absorbent polymer particles at a certain time. The data are automatically captured every 10 seconds. The measurement is carried out at 0.3 psi (21.0 g/cm²) for a period of 120 minutes per sample. The total liquid uptake is the total amount of aqueous saline solution absorbed by each 26 g sample.

Porosity

The porosity of the water-absorbent polymer particles is calculated as follows:

$$Porosity = \frac{AUNL - CRC}{AUNL}$$

Bulk Density/Flow Rate

The bulk density (BD) and the flow rate (FR) of the water-absorbent polymer particles is determined by the EDANA recommended test method No. WSP 250.3 (11) “Gravimetric Determination of flow rate, Gravimetric Determination of Density”.

Extractables

The level of extractable constituents in the water-absorbent polymer particles is determined by the EDANA recommended test method No. WSP 470.2-05 “Extractables”.

### EXAMPLES

Preparation of the Fluid-Absorbent Polymer Particles

The following polymer particles are used:
HySorb® B7160S available from BASF SE, Ludwigshafen, Germany
SAP-X prepared as described in Example 2

### Example 1 Base Polymer

The process was performed in a concurrent spray drying plant with an integrated fluidized bed (27) and an external fluidized bed (29) as shown in FIG. 8. The cylindrical part of the spray dryer (5) had a height of 22 m and a diameter of 3.4 m. The internal fluidized bed (IFB) had a diameter of 3 m and a weir height of 0.25 m.

The drying gas was feed via a gas distributor (3) at the top of the spray dryer. The drying gas was partly recycled (drying gas loop) via a baghouse filter (9) and a condenser column (12). Instead of the baghouse filter (9) any other filter and/or cyclone can be used. The drying gas was nitrogen that comprises from 1% to 4% by volume of residual oxygen: Before start of polymerization the drying gas loop was filled with nitrogen until the residual oxygen was below 4% by volume. The gas velocity of the drying gas in the cylindrical part of the spray dryer (5) was 0.82 m/s. The pressure inside the spray dryer was 4 mbar below ambient pressure.

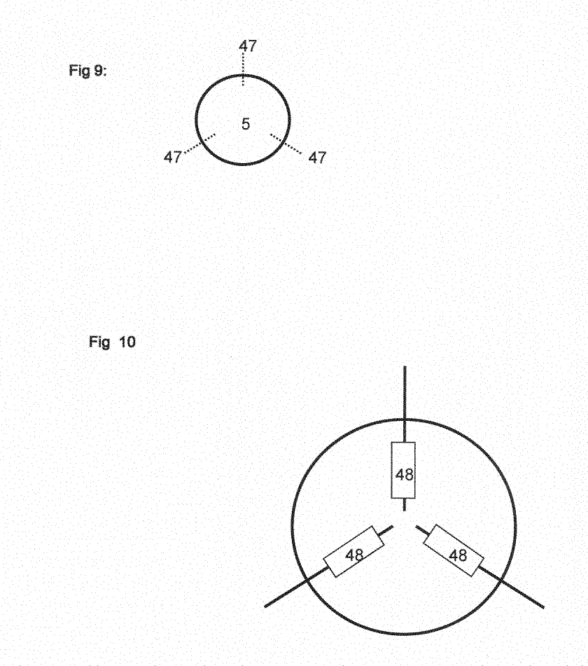

The spray dryer outlet temperature was measured at three points around the circumference at the end of the cyclindrical part as shown in FIG. 9. Three single measurements (47) were used to calculate the average cylindrical spray dryer outlet temperature. The drying gas loop was heated up and the dosage of monomer solution is started up. From this time the spray dryer outlet temperature was controlled to 118° C. by adjusting the gas inlet temperature via the heat exchanger (20). The gas inlet temperature was 167° C. and the steam content of the drying gas was 0.058 kg steam per kg dry gas.

The product accumulated in the internal fluidized bed (27) until the weir height was reached. Conditioned internal fluidized bed gas having a temperature of 104° C. and a steam content of 0.058 or 0.130 kg steam per kg dry gas was fed to the internal fluidized bed (27) via line (25). The gas velocity of the internal fluidized bed gas in the internal fluidized bed (27) was 0.65 m/s. The residence time of the product was 150 min. The temperature of the water-absorbent polymer particles in the internal fluidized bed was 82° C.

The spray dryer offgas was filtered in baghouse filter (9) and sent to a condenser column (12) for quenching/cooling. Excess water was pumped out of the condenser column (12) by controlling the (constant) filling level inside the condenser column (12). The water inside the condenser column (12) was cooled by a heat exchanger (13) and pumped counter-current to the gas via quench nozzles (11) so that the temperature inside the condenser column (12) was 45° C. The water inside the condenser column (12) was set to an alkaline pH by dosing sodium hydroxide solution to wash out acrylic acid vapors.

The condenser column offgas was split to the drying gas inlet pipe (1) and the conditioned internal fluidized bed gas (25). The gas temperatures were controlled via heat exchangers (20) and (22). The hot drying gas was fed to the concurrent spray dryer via gas distributor (3). The gas distributor (3) consists of a set of plates providing a pressure drop of 2 to 4 mbar depending on the drying gas amount.

The product was discharged from the internal fluidized bed (27) via rotary valve (28) into sieve (29). The sieve (29) was used for sieving off overs/lumps having a particle diameter of more than 800 μm.

The monomer solution was prepared by mixing first acrylic acid with 3-tuply ethoxylated glycerol triacrylate (internal crosslinker) and secondly with 37.3% by weight sodium acrylate solution.

The temperature of the resulting monomer solution was controlled to 10° C. by using a heat exchanger and pumping in a loop. A filter unit having a mesh size of 250 μm was used in the loop after the pump. The initiators were metered into the monomer solution upstream of the dropletizer by means of static mixers (41) and (42) via lines (43) and (44) as shown in FIG. 8. Sodium peroxodisulfate solution having a temperature of 20° C. was added via line (43) and [2,2′-azobis[2-(2-imidazolin-2-yl)propane]dihydrochloride solution together with Bruggolite FF7 having a temperature of 5° C. was added via line (44). Each initiator was pumped in a loop and dosed via control valves to each dropletizer unit. A second filter unit having a mesh size of 140 μm was used after the static mixer (42). For dosing the monomer solution into the top of the spray dryer three dropletizer units were used as shown in FIG. 10.

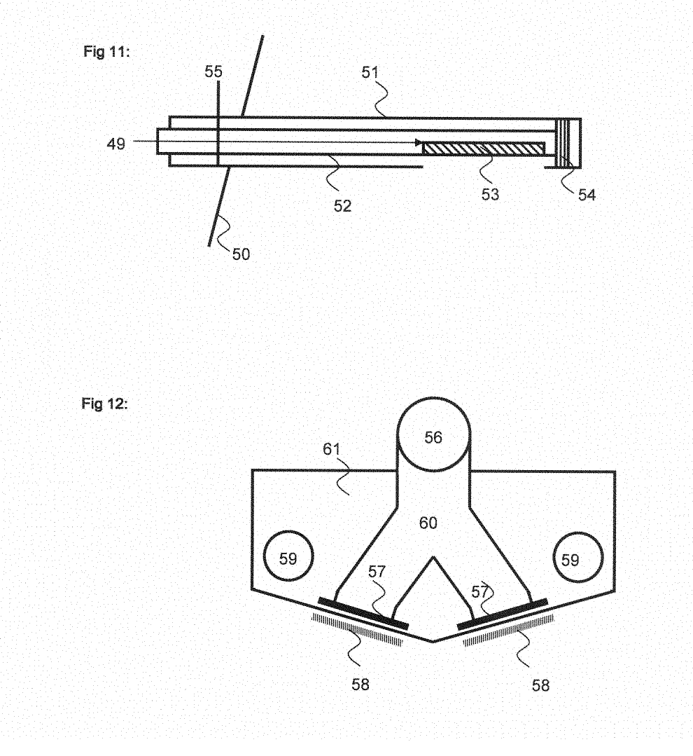

A dropletizer unit consisted of an outer pipe (51) having an opening for the dropletizer cassette (53) as shown in FIG. 11. The dropletizer cassette (53) was connected with an inner pipe (52). The inner pipe (53) having a PTFE block (54) at the end as sealing can be pushed in and out of the outer pipe (51) during operation of the process for maintenance purposes.

The temperature of the dropletizer cassette (61) was controlled to 8° C. by water in flow channels (59) as shown in FIG. 12. The dropletizer cassette (61) had 256 bores having a diameter of 170 μm and a bore separation of 15 mm. The dropletizer cassette (61) consisted of a flow channel (60) having essential no stagnant volume for homogeneous distribution of the premixed monomer and initiator solutions and one droplet plate (57). The droplet plate (57) had an angled configuration with an angle of 3°. The droplet plate (57) was made of stainless steel and had a length of 630 mm, a width of 128 mm and a thickness of 1 mm.

The feed to the spray dryer consisted of 9.56% by weight of acrylic acid, 33.73% by weight of sodium acrylate, 0.018% by weight of 3-tuply ethoxylated glycerol triacrylate, 0.036% by weight of [2,2′-azobis[2-(2-imidazolin-2-yl)propane]dihydrochloride, 0.0029% by weight of Bruggolite FF7, 0.072% by weight of sodium peroxodisulfate and water. The degree of neutralization was 73%. The feed per bore was 1.4 kg/h.

The resulting water-absorbent polymer particles were analyzed. The results are summarized in Table 1.

### Example 1B (Base Polymer)

The process was performed in a concurrent spray drying plant with an integrated fluidized bed (27) and an external fluidized bed (29) as shown in FIG. 8. The cylindrical part of the spray dryer (5) had a height of 22 m and a diameter of 3.4 m. The internal fluidized bed (IFB) had a diameter of 3 m and a weir height of 0.25 m.

The drying gas was feed via a gas distributor (3) at the top of the spray dryer. The drying gas was partly recycled (drying gas loop) via a baghouse filter (9) and a condenser column (12). Instead of the baghouse filter (9) any other filter and/or cyclone can be used. The drying gas was nitrogen that comprises from 1% to 4% by volume of residual oxygen: Before start of polymerization the drying gas loop was filled with nitrogen until the residual oxygen was below 4% by volume. The gas velocity of the drying gas in the cylindrical part of the spray dryer (5) was 0.82 m/s. The pressure inside the spray dryer was 4 mbar below ambient pressure. The spray dryer outlet temperature was measured at three points around the circumference at the end of the cyclindrical part as shown in FIG. 9. Three single measurements (47) were used to calculate the average cylindrical spray dryer outlet temperature. The drying gas loop was heated up and the dosage of monomer solution is started up. From this time the spray dryer outlet temperature was controlled to 118° C. by adjusting the gas inlet temperature via the heat exchanger (20). The gas inlet temperature was 167° C. and the steam content of the drying gas was 0.058 kg steam per kg dry gas.

The product accumulated in the internal fluidized bed (27) until the weir height was reached. Conditioned internal fluidized bed gas having a temperature of 104° C. and a steam content of 0.058 or 0.130 kg steam per kg dry gas was fed to the internal fluidized bed (27) via line (25). The gas velocity of the internal fluidized bed gas in the internal fluidized bed (27) was 0.65 m/s. The residence time of the product was 150 min. The temperature of the water-absorbent polymer particles in the internal fluidized bed was 82° C.

The spray dryer offgas was filtered in baghouse filter (9) and sent to a condenser column (12) for quenching/cooling. Excess water was pumped out of the condenser column (12) by controlling the (constant) filling level inside the condenser column (12). The water inside the condenser column (12) was cooled by a heat exchanger (13) and pumped counter-current to the gas via quench nozzles (11) so that the temperature inside the condenser column (12) was 45° C. The water inside the condenser column (12) was set to an alkaline pH by dosing sodium hydroxide solution to wash out acrylic acid vapors.

The condenser column offgas was split to the drying gas inlet pipe (1) and the conditioned internal fluidized bed gas (25). The gas temperatures were controlled via heat exchangers (20) and (22). The hot drying gas was fed to the concurrent spray dryer via gas distributor (3). The gas distributor (3) consists of a set of plates providing a pressure drop of 2 to 4 mbar depending on the drying gas amount.

The product was discharged from the internal fluidized bed (27) via rotary valve (28) into sieve (29). The sieve (29) was used for sieving off overs/lumps having a particle diameter of more than 800 μm.

The monomer solution was prepared by mixing first acrylic acid with 3-tuply ethoxylated glycerol triacrylate (internal crosslinker) and secondly with 37.3% by weight sodium acrylate solution.

The temperature of the resulting monomer solution was controlled to 10° C. by using a heat exchanger and pumping in a loop. A filter unit having a mesh size of 250 μm was used in the loop after the pump. The initiators were metered into the monomer solution upstream of the dropletizer by means of static mixers (41) and (42) via lines (43) and (44) as shown in FIG. 1. Sodium peroxodisulfate solution having a temperature of 20° C. was added via line (43) and [2,2′-azobis[2-(2-imidazolin-2-yl)propane]dihydrochloride solution together with Brüggolite FF7 having a temperature of 5° C. was added via line (44). Each initiator was pumped in a loop and dosed via control valves to each dropletizer unit. A second filter unit having a mesh size of 140 μm was used after the static mixer (42). For dosing the monomer solution into the top of the spray dryer three dropletizer units were used as shown in FIG. 4.

A dropletizer unit consisted of an outer pipe (51) having an opening for the dropletizer cassette (53) as shown in FIG. 5. The dropletizer cassette (53) was connected with an inner pipe (52). The inner pipe (53) having a PTFE block (54) at the end as sealing can be pushed in and out of the outer pipe (51) during operation of the process for maintenance purposes.

The temperature of the dropletizer cassette (61) was controlled to 8° C. by water in flow channels (59) as shown in FIG. 6. The dropletizer cassette (61) had 256 bores having a diameter of 170 μm and a bore separation of 15 mm. The dropletizer cassette (61) consisted of a flow channel (60) having essential no stagnant volume for homogeneous distribution of the premixed monomer and initiator solutions and one droplet plate (57). The droplet plate (57) had an angled configuration with an angle of 3°. The droplet plate (57) was made of stainless steel and had a length of 630 mm, a width of 128 mm and a thickness of 1 mm.

The feed to the spray dryer consisted of 9.56% by weight of acrylic acid, 33.73% by weight of sodium acrylate, 0.023% by weight of 3-tuply ethoxylated glycerol triacrylate, 0.072% by weight of [2,2′-azobis[2-(2-imidazolin-2-yl)propane]dihydrochloride, 0.054% by weight of Blancolen® HP, 0.0029% by weight of Bruggolite FF7, 0.090% by weight of sodium peroxodisulfate and water. The degree of neutralization was 73%. The feed per bore was 1.4 kg/h.

The resulting water-absorbent polymer particles were analyzed. The results are summarized in Table 1.

TABLE 1
Base polymers

| | Steam Content | Bulk Density | CRC | AUL | Residual Monomers | Extractables | Moisture |
|---|---|---|---|---|---|---|---|
| Example | [kg/kg] | [g/cm³] | [g/g] | [g/g] | [ppm] | [wt. %] | [wt. %] |
| 1 | 0.130 | 0.70 | 50.4 | 9.8 | 3600 | 7.7 | 9.3 |
| 1B | 0.130 | 0.68 | 50.5 | 8.8 | 4200 | 7.5 | 7.6 |

### Example 2 Surface Crosslinked Water-Absorbent Polymer Particles

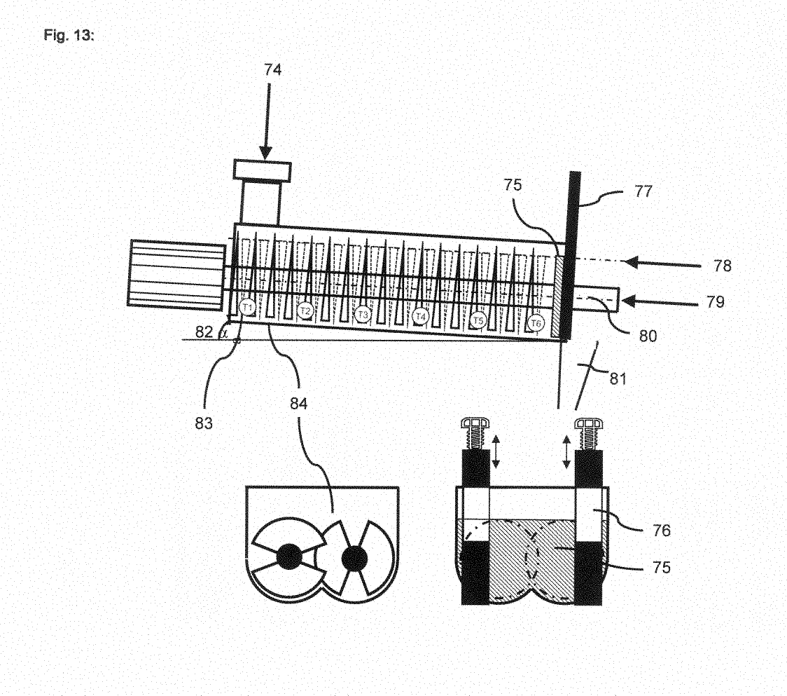

In a Schugi Flexomix® (model Flexomix-160, manufactured by Hosokawa Micron B.V., Doetinchem, the Netherlands) with a speed of 2000 rpm, the base polymer (Example 1) was coated with a surface-postcrosslinker solution by using 2 or 3 round spray nozzle systems (model Gravity-Fed Spray Set-ups, External Mix Typ SU4, Fluid Cap 60100 and Air Cap SS-120, manufactured by Spraying Systems Co, Wheaton, Ill., USA) and then filled via inlet (74) and dried in a NARA heater (model NPD 5W-18, manufactured by GMF Gouda, Waddinxveen, the Netherlands) with a speed of the shaft (80) of 6 rpm. The NARA heater has two paddles with a shaft offset of 90° (84) and a fixed discharge zone (75) with two flexible weir plates (77). Each weir has a weir opening with a minimal weir height at 50% (79) and a maximal weir opening at 100% (78) as shown in FIG. 13.

The inclination angle α (82) between the floor plate and the NARA paddle dryer is approx. 3°. The weir height of the NARA heater is between 50 to 100% corresponding to a residence time of approx. 40 to 150 min, by a product density of approx. 700 to 750 kg/m³. The final product temperature in the NARA heater is 145° C. After drying, the surface-postcrosslinked base polymer was transported over discharge cone (81) in the NARA cooler (GMF Gouda, Waddinxveen, the Netherlands), to cool down the surface postcrosslinked base polymer to approx. 60° C. with a speed of 11 rpm and a weir height of 145 mm. After cooling, the material was sieved with a minimum cut size of 150 μm and a maximum size cut of 710 μm.

Ethylene carbonate, water, Plantacare® UP 818 (BASF SE, Ludwigshafen, Germany) and aqueous aluminum lactate (26% by weight) was premixed and spray coated as summarized in Tab 6. As aluminum lactate, Lothragon® A1 220 (manufactured by Dr. Paul Lohmann GmbH, Emmerthal, Germany) was used.

The metered amounts and conditions of the coating into the Schugi Flexomix®, the conditions, the formulation and values of the drying and cooling step are summarized in Table 2 to 3:

5.0 wt % of a 0.1% aqueous solution of Plantacare® 818 UP (BASF SE, Ludwigshafen, Germany) having a temperature of approx. 25° C. was additionally added into the cooler using two nozzles in the first third of the cooler. The nozzles were placed below the product bed.

All physical properties of the resulting polymers (SAP-X) are summarized in Table 4 and 5:

TABLE 2
Process parameters of the thermal treatment in the heater

| | Product | | | | | | | | | | | | |
|---|---|---|---|---|---|---|---|---|---|---|---|---|---|
| | Temp. | Steam | Steam | | | | | | | | | | |
| | Set | Pressure | Pressure | Heater | Heater | Heater | Heater | Heater | Heater | Through- | Heater | | |
| | Value | Wave | Jacket | T1 | T2 | T3 | T4 | T5 | T6 | put | Weir | No. of | Pos. of |
| Example | ° C. | bar | bar | ° C. | ° C. | ° C. | ° C. | ° C. | ° C. | kg/h | % | Nozzles | Nozzles |
| 2 | 145 | 5.0 | 5.0 | 93 | 99 | 118 | 128 | 138 | 145 | 470 | 80 | 3 | 90/180/270° |
| (SAP-X) | | | | | | | | | | | | | |

TABLE 3
Surface-postcrosslinker formulation of the thermal treatment
in the heater and remoistening in the cooler

| | | SXL | | | | Cooler | |
|---|---|---|---|---|---|---|---|
| | | | | Plantacare | Al-Lactate | Plantacare | |
| | Base | EC | Water | 818 UP | (dry) | 818 UP | Water |
| Example | polymer | bop % | bop % | bop ppm | bop % | bop ppm | bop % |
| 2 | Base Polymer | 2.0 | 5.0 | 25 | 0.1 | 25 | 5.0 |
| (SAP-X) | (Example 1) | | | | | | |

EC: Ethylene carbonate; bop: based on polymer

TABLE 4
Physical properties of the polymer particles after surface-postcrosslinking

| | | | | | | | | | Residual | | | |
|---|---|---|---|---|---|---|---|---|---|---|---|---|
| | CRC | AUNL | AUL | AUHL | SFC | Vortex | FSR | Moisture | Monomers | Extractables | Bulk Density | FR |
| Example | g/g | g/g | g/g | g/g | 10⁻⁷ cm³ · s/g | S | g/g · s | % | ppm | % | g/100 ml | g/s |
| 2 | 42.0 | 57.9 | 35.1 | 25.1 | 1 | 69 | 0.28 | 4.5 | 361 | 4.1 | 86 | 17 |

TABLE 5
Particle size distribution of the polymer particles after surface-postcrosslinking - Sieve fractions

| | <106 μm | >106 μm | >200 μm | >250 μm | >300 μm | >400 μm | >500 μm | >600 μm | >710 μm |
|---|---|---|---|---|---|---|---|---|---|
| Example | % | % | % | % | % | % | % | % | % |
| 2 | 0.0 | 0.3 | 2.3 | 21.4 | 53.3 | 20.9 | 1.6 | 0.2 | 0.1 |

The characteristics of the fluid-absorbent polymer particles are summarized in Tab. 6

TABLE 6
Summary of the characteristics of the SAP used in the examples 3 to 16

| | | | | | | | | Residual | |
|---|---|---|---|---|---|---|---|---|---|
| | CRC | AUL | AUHL | SFC | Vortex | Moisture | FSR | monomers | extractables |
| | (g/g) | (g/g) | (g/g) | 10−7 cm³ *s/g | Sec | wt. % | g/g*s | Ppm | wt. % |
| Example 2 | 42.0 | 35.1 | 25.1 | 1 | 69 | 4.5 | 0.28 | 361 | 4.1 |
| (SAP-X) | | | | | | | | | |
| Hysorb ® | 29.5 | 28 | 23 | 36 | 116 | 3.3 | 0.17 | 295 | 9 |
| B7160S | | | | | | | | | |

TABLE 7
Geometry of the acquisition-distribution layers

| | | | | | Hole (aperture) area | | | total hole | total film | | |
|---|---|---|---|---|---|---|---|---|---|---|---|
| | | Segment | | | female | male | | area per | area per | total hole | segment |
| | | width | height | width/ | side | side | female/ | ADL surface | ADL surface | area/total | width/hole |
| | matrix type | μm | μm | height | mm² | mm² | male | % | % | film area | width |
| HEX 26 | plastic film | 1252 | 1048 | 1.2 | 0.78 | 0.67 | 1.2 | 27% | 73% | 0.4 | 1.3 |
| Ellipse 26 | plastic film | 380 | 485 | 0.8 | 0.58 | 0.43 | 1.3 | 54% | 46% | 1.2 | 0.8 |
| AquiDry | plastic film | 372 | 500 | 0.7 | 0.33 | 0.22 | 1.5 | 50% | 50% | 1.0 | 0.6 |
| N-Hance | non woven | 1360 | 1192 | 1.1 | 3.28 | 1.22 | 2.7 | 36% | 64% | 0.6 | 0.7 |

The Geometry, the height and width of the segments of the acquisition-distribution layer and the area of the apertures (hole area) are determined by using a digital video microscope, Keyence VHX-100 K (Keyence Corporation, Japan).

Approximately 5 mm wide stripes of the ADL film are cut through the center of the openings (apertures) of the acquisition-distribution layer using a sharp scissor. The section of the acquisition-distribution layer is then fixed between two microscope slides, to have the cut section in perpendicular direction to the lens of the microscope.

Then the section of the acquisition-distribution layer is aligned under the microscope's lens by using the x and y-axis positioning table. Lens magnification of ×75 is used for the analysis.

Finally photos of the acquisition-distribution layer cross section are taken and measured by selecting the desired regions and spaces in the photo.

### Example 3 Fluid-Absorbent Pad

The fluid-absorbent pad consists of a single core system having a rectangular size of 41 cm×10 cm. The fluid-absorbent pad comprises a multi-layered system of spunbond layer coverstock as top sheet (A), three dimensional film as acquisition distribution layer (D) and fluid absorbent core (C) made of fluff/SAP mixtures.

The total weight of fluff pulp (cellulose fibers) is 7 g. The density of the fluid-absorbent core is in average 0.25-0.30 g/cm3. The basis weight of the core is 465 gsm. The fluid-absorbent core holds 65% by weight uniformly distributed fluid-absorbent polymer particles (SAP-X); the quantity of fluid-absorbent polymer particles within the fluid-absorbent core is 13 g.

The absorbent core is covered with a tissue, as a core wrap having a basis weight of 18 gsm.

The three dimensional polyethylene film used as acquisition-distribution layer, N-Sorb HEX 26 (Neos Italia Srl, S. Giovanni Teatino, Italy) has a basis weight of 26 gsm. The acquisition-distribution layer is rectangular shaped of a size of 20 cm×9 cm and is placed in on top of the fluid absorbent core with the rough surface towards coverstock (A).

The water-absorbent polymer particles derived from dropletization polymerization as described in Example 2 (SAP-X) exhibiting the following features and absorption profile as listed in Table 6:

CRC of 42.0 g/g

SFC of 1×10⁻⁷ cm³s/g

AUHL of 25.1 g/g

AUL of 35.1 g/g

Extractables of 4.1 wt. %

Residual monomers of 361 ppm

Moisture content of 4.5 wt. %

FSR of 0.28 g/g*s

Vortex of 69 sec

Acquisition time and rewet value of the fluid absorbent pad are determined and results are summarized in Table 8 and 9.

### Example 4

A fluid-absorbent pad of Example 3 was repeated, except that the quantity of fluid-absorbent polymer particles (SAP-X) within the core was reduced to 9 g. The density of the fluid-absorbent core is in average 0.25-0.30 g/cm³. The basis weight of the fluid-absorbent core is in average 370 gsm. The fluid-absorbent core holds 56% by weight uniformly distributed fluid-absorbent polymer particles.

Acquisition time and rewet value of the fluid absorbent pad are determined and results are summarized in Table 8

### Example 5

A fluid-absorbent pad of example 3 was repeated, except that the three dimensional nowoven film, N-Sorb N-Hance34SB7 (Neos Italia Srl, S. Giovanni Teatino, Italy) of a basis weight of 34 gsm was used as acquisition-distribution layer. The acquisition-distribution layer is rectangular shaped of a size of 20 cm×9 cm and is placed in on top of the fluid absorbent core with the rough surface towards coverstock (A).

Acquisition time and rewet value of the fluid absorbent pad are determined and results are summarized in Table 8 and 9.

### Example 6

A fluid-absorbent pad of example 5 was repeated, except that the quantity of fluid-absorbent polymer particles (SAP-X) within the core was reduced to 9 g. The density of the fluid-absorbent core is in average 0.25-0.30 g/cm³. The basis weight of the fluid-absorbent core is 370 gsm. The fluid-absorbent core holds 56% by weight uniformly distributed fluid-absorbent polymer particles.

Acquisition time and rewet value of the fluid absorbent pad are determined and results are summarized in Table 8.

### Example 7—Comparative

A fluid-absorbent pad of example 3 was repeated, except that a fluid absorbent polymer particles prepared in example 2 were replaced by the commercially available Hysorb® B7160S (BASF SE, Ludwigshafen, Germany)

Hysorb® B7160S consists of irregular shaped fluid-absorbent polymer particles produced by gel polymerization exhibiting the following features and absorption profile

CRC of 30 g/g

SFC of 36 10⁻⁷ cm³s/g

AUHL of 23 g/g

AUL of 28 g/g

Extractables of 9 wt. %

Residual monomers of 295 ppm

Moisture content of 3.3 wt. %

FSR of 0.17 g/g*s

Vortex of 116 sec

Acquisition time and rewet value of the fluid absorbent pad are determined and results are summarized in Table 8.

### Example 8—Comparative

A fluid-absorbent pad of example 5 was repeated, except that that a fluid absorbent polymer particles prepared in example 2 were replaced by the commercially available Hysorb® B7160S (BASF SE, Ludwigshafen, Germany)

Acquisition time and rewet value of the fluid absorbent pad are determined and results are summarized in Table 8.

The examples demonstrate that the fluid-absorbent cores comprising SAP-X in combination with 3D perforated ADL show better rewet and liquid acquisition time, even when the loading of SAP-X in the absorbent core is reduced up to 30%, in comparison to the fluid-absorbent core containing irregular shaped SAP (Hysorb® B7160S).

The examples demonstrate that the fluid-absorbent cores comprising SAP-X in combination with 3D perforated ADL show similar rewet and better liquid acquisition time, even when the loading of SAP-X in the absorbent core is reduced up to 30%, in comparison to the fluid-absorbent core containing SAP-X in combination with air-through bonded non-woven ADL.

### Example 9—Comparative

The fluid-absorbent pad consists of a single core system having a rectangular size of 41 cm×10 cm. The fluid-absorbent pad comprises a multi-layered system of spunbond layer coverstock as top sheet (A), an air-through bonded acquisition distribution layer (D) and fluid absorbent core (C) made of fluff/SAP mixtures.

The total weight of fluff pulp (cellulose fibers) is 7 g. The density of the fluid-absorbent core is in average 0.25-0.30 g/cm3. The basis weight of the core is 465 gsm. The fluid-absorbent core holds 65% by weight uniformly distributed fluid-absorbent polymer particles Hysorb® B7160S (BASF SE, Ludwigshafen, Germany); the quantity of fluid-absorbent polymer particles within the fluid-absorbent core is 13 g.

The absorbent core may be wrapped or covered with a tissue, having a basis weight of 18 gsm.

The air-through bonded nonwoven used as acquisition-distribution layer (Multifunctional Acquitex, Texus, Italy) has a basis weight of 40 gsm. The acquisition-distribution layer is rectangular shaped of a size of 20 cm×9 cm and is placed in on top of the fluid absorbent core.

Acquisition time and rewet value of the fluid absorbent pad are determined and results are summarized in Table 8, 9 and 10.

### Example 10

A fluid-absorbent pad of example 3 was repeated, except that the three dimensional film N-Sorb Ellipse 26, (Neos Italia Srl, S. Giovanni Teatino, Italy) of a basis weight of 26 gsm was used as acquisition-distribution layer. The acquisition-distribution layer is rectangular shaped of a size of 20 cm×9 cm and is placed in on top of the fluid absorbent core with the rough surface towards coverstock (A).

Acquisition time and rewet value of the fluid absorbent pad are determined and results are summarized in Table 9

### Example 11

A fluid-absorbent pad of example 3 was repeated, except that the three dimensional polyethylene film (AquiDry™ Plus, Tredegar Film Products Corporation) of a basis weight of 26 gsm was used as acquisition-distribution layer. The acquisition-distribution layer is rectangular shaped of a size of 20 cm×9 cm and is placed in on top of the fluid absorbent core with the rough surface towards coverstock (A).

Acquisition time and rewet value of the fluid absorbent pad are determined and results are summarized in Table 9.

TABLE 8
Rewet under load, acquisition times and rates for each liquid insult into the pads

| | | SAP_Cot | SAP | Fluff | ADL | capacity | REWET UNDER LOAD [g] | | | | |
|---|---|---|---|---|---|---|---|---|---|---|---|
| Example | SAP. type | [%] | [g] | [g] | type | [g/g] | Rewet 1 | Rewet 2 | Rewet 3 | Rewet 4 | Rewet 5 |
| 3 | SAP-X | 65 | 13 | 7 | N-Sorb HEX26 | 562 | 0.10 | 0.10 | 0.10 | 0.12 | 0.26 |
| | | | | | “flipside” | | | | | | |
| 4 | SAP-X | 56 | 9 | 7 | N-Sorb HEX26 | 395 | 0.08 | 0.06 | 0.33 | 1.70 | 3.48 |
| | | | | | “flipside” | | | | | | |
| 5 | SAP-X | 65 | 13 | 7 | N-Sorb N- | 562 | 0.08 | 0.09 | 0.12 | 0.37 | 0.56 |
| | | | | | Hance34SB7 | | | | | | |
| | | | | | “flipside” | | | | | | |
| 6 | SAP-X | 56 | 9 | 7 | N-Sorb N- | 395 | 0.07 | 0.09 | 0.24 | 1.90 | 3.59 |
| | | | | | Hance34SB7 | | | | | | |
| | | | | | “flipside” | | | | | | |
| 7 | B7160S | 65 | 13 | 7 | N-Sorb HEX26 | 395 | 0.15 | 0.11 | 0.48 | 2.29 | 8.49 |
| | | | | | “flipside” | | | | | | |
| 8 | B7160S | 65 | 13 | 7 | N-Sorb N- | 466 | 0.05 | 0.07 | 1.28 | 4.90 | 6.04 |
| | | | | | Hance34SB7 | | | | | | |
| | | | | | “flipside” | | | | | | |
| 9 | B7160S | 65 | 13 | 7 | air-through | 395 | 0.08 | 0.10 | 2.50 | 5.51 | 9.16 |
| | | | | | 40 gsm | | | | | | |
| 12 | SAP-X | 65 | 13 | 7 | air-through | 562 | 0.08 | 0.08 | 0.25 | 0.87 | 3.02 |
| | | | | | 40 gsm | | | | | | |

| | Liquid Acquisition Time [sec] | | | | | Liquid Acquisition Rate [g/sec] | | | | |
|---|---|---|---|---|---|---|---|---|---|---|
| Example | A1 | A2 | A3 | A4 | A5 | ACQ1 | ACQ2 | ACQ3 | ACQ4 | ACQ5 |
| 3 | 17 | 19 | 23 | 27 | 31 | 5.88 | 2.70 | 2.17 | 1.89 | 1.64 |
| 4 | 20 | 23 | 30 | 37 | 46 | 4.94 | 2.15 | 1.69 | 1.36 | 1.09 |
| 5 | 35 | 45 | 55 | 67 | 79 | 2.86 | 1.12 | 0.90 | 0.75 | 0.64 |
| 6 | 32 | 46 | 53 | 55 | 55 | 3.10 | 1.10 | 0.95 | 0.92 | 0.90 |
| 7 | 21 | 24 | 51 | 102 | 156 | 4.76 | 2.08 | 0.98 | 0.49 | 0.32 |
| 8 | 49 | 90 | 112 | 128 | 141 | 2.03 | 0.55 | 0.45 | 0.39 | 0.35 |
| 9 | 101 | 211 | 229 | 246 | 263 | 0.99 | 0.24 | 0.22 | 0.20 | 0.19 |
| 12 | 77 | 102 | 117 | 127 | 139 | 1.30 | 0.49 | 0.43 | 0.40 | 0.36 |

TABLE 9
Rewet under load, acquisition times and rates for each liquid insult into the pads
covered with three dimensional ADLs male side facing upper layer (“flipside”)

| | | SAP_Cot | SAP | Fluff | ADL | capacity | REWET UNDER LOAD [g] | | | | |
|---|---|---|---|---|---|---|---|---|---|---|---|
| Example | SAP. type | [%] | [g] | [g] | type | [g/g] | Rewet 1 | Rewet 2 | Rewet 3 | Rewet 4 | Rewet 5 |
| 3 | SAP-X | 65 | 13 | 7 | N-Sorb HEX26 | 562 | 0.10 | 0.10 | 0.10 | 0.12 | 0.26 |
| | | | | | “flipside” | | | | | | |
| 17 | base | 65 | 13 | 7 | N-Sorb HEX26 | 657 | 0.11 | 0.09 | 0.08 | 0.10 | 0.18 |
| | polymer | | | | “flipside” | | | | | | |
| 10 | SAP-X | 65 | 13 | 7 | N-Sorb | 562 | 0.18 | 0.20 | 0.17 | 0.18 | 0.30 |
| | | | | | Ellipse26 | | | | | | |
| | | | | | “flipside” | | | | | | |
| 5 | SAP-X | 65 | 13 | 7 | Hance34SB7 | 562 | 0.08 | 0.09 | 0.12 | 0.37 | 0.56 |
| | | | | | “flipside” | | | | | | |
| 11 | SAP-X | 65 | 13 | 7 | AquiDryPlus | 562 | 0.18 | 0.18 | 0.13 | 0.24 | 0.30 |
| | | | | | “flipside” | | | | | | |
| 12 | SAP-X | 65 | 13 | 7 | air-through | 562 | 0.08 | 0.08 | 0.25 | 0.87 | 3.02 |
| | | | | | 40 gsm | | | | | | |
| 9 | B7160S | 65 | 13 | 7 | air-through | 395 | 0.08 | 0.10 | 2.50 | 5.51 | 9.16 |
| | | | | | 40 gsm | | | | | | |

| | Liquid Acquisition Time [sec] | | | | | Liquid Acquisition Rate [g/sec] | | | | |
|---|---|---|---|---|---|---|---|---|---|---|
| Example | A1 | A2 | A3 | A4 | A5 | ACQ1 | ACQ2 | ACQ3 | ACQ4 | ACQ5 |
| 3 | 17 | 19 | 23 | 27 | 31 | 5.88 | 2.70 | 2.17 | 1.89 | 1.64 |
| 17 | 19 | 17 | 21 | 24 | 28 | 5.41 | 2.99 | 2.44 | 2.08 | 1.82 |
| 10 | 34 | 34 | 41 | 51 | 67 | 2.94 | 1.47 | 1.22 | 0.98 | 0.75 |
| 5 | 35 | 45 | 55 | 67 | 79 | 2.86 | 1.12 | 0.90 | 0.75 | 0.64 |
| 11 | 50 | 53 | 49 | 58 | 69 | 2.02 | 0.95 | 1.03 | 0.87 | 0.72 |
| 12 | 77 | 102 | 117 | 127 | 139 | 1.30 | 0.49 | 0.43 | 0.40 | 0.36 |
| 9 | 101 | 211 | 229 | 246 | 263 | 0.99 | 0.24 | 0.22 | 0.20 | 0.19 |

### Example 12—Comparative

The fluid-absorbent pad consists of a single core system having a rectangular size of 41 cm×10 cm. The fluid-absorbent pad comprises a multi-layered system of spunbond layer coverstock as top sheet (A), an air-through bonded acquisition distribution layer (D) and fluid absorbent core (C) made of fluff/SAP mixtures.

The total weight of fluff pulp (cellulose fibers) is 7 g. The density of the fluid-absorbent core is in average 0.25-0.30 g/cm³. The basis weight of the core is 465 gsm. The fluid-absorbent core holds 65% by weight uniformly distributed fluid-absorbent polymer particles prepared as described in example 2 (SAP-X); the quantity of fluid-absorbent polymer particles within the fluid-absorbent core is 13 g.

The absorbent core may be wrapped or covered with a tissue, having a basis weight of 18 gsm.

The air-through bonded nonwoven used as acquisition-distribution layer (Multifunctional Acquitex, Texus, Italy) has a basis weight of 40 gsm. The acquisition-distribution layer is rectangular shaped of a size of 20 cm×9 cm and is placed in on top of the fluid absorbent core.

Acquisition time and rewet value of the fluid absorbent pad are determined and results are summarized in Tables 8, 9 and 10.

### Example 13

The fluid-absorbent pad consists of a single core system having a rectangular size of 41 cm×10 cm. The fluid-absorbent pad comprises a multi-layered system of spunbond layer coverstock as top sheet (A), three dimensional film as acquisition distribution layer (D) and fluid absorbent core (C) made of fluff/SAP mixtures.

The total weight of fluff pulp (cellulose fibers) is 7 g. The density of the fluid-absorbent core is in average 0.25-0.30 g/cm³. The basis weight of the core is 465 gsm. The fluid-absorbent core holds 65% by weight uniformly distributed fluid-absorbent polymer particles; the quantity of fluid-absorbent polymer particles within the fluid-absorbent core is 13 g.

The absorbent core is covered with a tissue, as a core wrap having a basis weight of 18 gsm.

The three dimensional polyethylene film used as acquisition-distribution layer (AquiDry™ Plus, Tredeger Film Products Corporation) has a basis weight of 26 gsm. The acquisition-distribution layer is rectangular shaped of a size of 20 cm×9 cm and is placed in on top of the fluid absorbent core with the smooth surface towards coverstock (A). The water-absorbent polymer particles derived from droplet polymerization as described in example 2 (SAP X), exhibiting the following features and absorption profile:

CRC of 42.0 g/g

SFC of 1×10⁻⁷ cm³s/g

AUHL of 25.1 g/g

AUL of 35.1 g/g

Extractables of 4.1 wt. %

Residual monomers of 361 ppm

Moisture content of 4.5 wt. %

FSR of 0.28 g/g*s

Vortex of 69 sec

PSD of 150 to 710 μm

Acquisition time and rewet value of the fluid absorbent pad are determined and results are summarized in Table 10

### Example 14

A fluid-absorbent pad of example 13 was repeated, except that the three dimensional film, N-Sorb Ellipse 26 (Neos Italia Srl, S. Giovanni Teatino, Italy) of a basis weight of 26 gsm was used as acquisition-distribution layer. The acquisition-distribution layer is rectangular shaped of a size of 20 cm×9 cm and is placed in on top of the fluid absorbent core with the smooth surface towards coverstock (A).

Acquisition time and rewet value of the fluid absorbent pad are determined and results are summarized in Table 10

### Example 15

A fluid-absorbent pad of example 13 was repeated, except that the three dimensional nowoven film, N-Sorb N-hance34SB7 (Neos Italia Srl, S. Giovanni Teatino, Italy) of a basis weight of 34 gsm was used as acquisition-distribution layer. The acquisition-distribution layer is rectangular shaped of a size of 20 cm×9 cm and is placed in on top of the fluid absorbent core with the smooth surface towards coverstock (A).

Acquisition time and rewet value of the fluid absorbent pad are determined and results are summarized in Table 10

### Example 16

A fluid-absorbent pad of example 13 was repeated; except that the three dimensional film N-Sorb HEX26, (Neos Italia Srl, S. Giovanni Teatino, Italy) of a basis weight of 26 gsm was used as an acquisition-distribution layer. The acquisition-distribution layer is rectangular shaped of a size of 20 cm×9 cm and is placed in on top of the fluid absorbent core with the smooth surface towards coverstock (A).

Acquisition time and rewet value of the fluid absorbent pad are determined and results are summarized in Table 10.

### Example 17

A fluid-absorbent pad of example 3 was repeated; except that the fluid absorbent polymer particles used for the pad preparation were base polymer particles derived from droplet polymerization as described in example 1B, exhibiting the features and absorption profile as listed in table 1B.

Acquisition time and rewet value of the fluid absorbent pad are determined and results are summarized in Table 9.

TABLE 10
Rewet under load, acquisition times and rates for each liquid insult into the pads
covered with three dimensional ADLs female side facing upper layer (“regular side”)

| | | SAP_Cot | SAP | Fluff | ADL | capacity | REWET UNDER LOAD [g] | | | | |
|---|---|---|---|---|---|---|---|---|---|---|---|
| Example | SAP. type | [%] | [g] | [g] | type | [g/g] | Rewet 1 | Rewet 2 | Rewet 3 | Rewet 4 | Rewet 5 |
| 13 | SAP-X | 65 | 13 | 7 | AquiDryPlus | 562 | 0.14 | 0.18 | 0.13 | 0.15 | 0.23 |
| | | | | | “regular side” | | | | | | |
| 14 | SAP-X | 65 | 13 | 7 | N-Sorb | 562 | 0.21 | 0.17 | 0.20 | 0.37 | 0.47 |
| | | | | | Ellipse26 | | | | | | |
| | | | | | “regular side” | | | | | | |
| 15 | SAP-X | 65 | 13 | 7 | N-Sorb N- | 562 | 0.05 | 0.06 | 0.29 | 1.00 | 2.04 |
| | | | | | Hance34SB7 | | | | | | |
| | | | | | “regular side” | | | | | | |
| 16 | SAP-X | 65 | 13 | 7 | N-Sorb HEX26 | 562 | 0.21 | 0.10 | 0.06 | 0.68 | — |
| | | | | | “regular side” | | | | | | |
| 12 | SAP-X | 65 | 13 | 7 | air-through | 562 | 0.08 | 0.08 | 0.25 | 0.87 | 3.02 |
| | | | | | 40 gsm | | | | | | |
| 9 | B7160S | 65 | 13 | 7 | air-through | 395 | 0.08 | 0.10 | 2.50 | 5.51 | 9.16 |
| | | | | | 40 gsm | | | | | | |

| | Liquid Acquisition Time [sec] | | | | | Liquid Acquisition Rate [g/sec] | | | | |
|---|---|---|---|---|---|---|---|---|---|---|
| Example | A1 | A2 | A3 | A4 | A5 | ACQ1 | ACQ2 | ACQ3 | ACQ4 | ACQ5 |
| 13 | 31 | 39 | 51 | 57 | 65 | 3.23 | 1.30 | 0.99 | 0.88 | 0.77 |
| 14 | 32 | 32 | 34 | 37 | 42 | 3.17 | 1.56 | 1.47 | 1.37 | 1.20 |
| 15 | 107 | 168 | 177 | 178 | 181 | 0.93 | 0.30 | 0.28 | 0.28 | 0.28 |
| 16 | 64 | 167 | 327 | 480 | — | 1.56 | 0.30 | 0.15 | 0.10 | — |
| 12 | 77 | 102 | 117 | 127 | 139 | 1.30 | 0.49 | 0.43 | 0.40 | 0.36 |
| 9 | 101 | 211 | 229 | 246 | 263 | 0.99 | 0.24 | 0.22 | 0.20 | 0.19 |

1. Absorbent article, comprising
(A) an upper liquid-pervious layer,
(B) a lower liquid-impervious layer,
(C) a fluid-absorbent core between the layer (A) and the layer (B),
comprising from 0 to 90% by weight fibrous material and from 10 to 100% by weight water-absorbent polymer particles having a mean sphericity (SPHT) from 0.8 to 0.95, based on the sum of water-absorbent polymer particles and fibrous material;
(D) an acquisition-distribution layer between (A) and (C) having a three dimensional apertured structure having a female side (5) and a male side (6), wherein the female side presents a multiplicity of openings extending in the form of through holes in the direction of a lower surface of the same structure being the male side,
(E) an optional tissue layer disposed immediately above and/or below (C); and
(F) other optional components,
wherein the quantity of the water absorbent polymer particles within the fluid absorbent core is at least 3 g.

2. The absorbent article according to claim 1, wherein mutually adjacent through holes of the acquisition-distribution layer (D) being separated by segments (10) of said acquisition-distribution layer (D) having a profile with symmetrical sides converging towards the female side.

3. The absorbent article according to claim 2, wherein the profile of the segments has a conic section as a cross section,

4. The absorbent article according to claim 1, wherein the base (8) with a length D1 of the segment is at the male side and the apex (9) at the female side.

5. The absorbent article according to claim 1, wherein the height (H1) of the segments is given by the distance of the base to the apex (9).

6. The absorbent article according to claim 1, wherein the width of the segments (W1) is given by the width of the cross-section at H1/2.

7. The absorbent article according to claim 1, wherein the through holes have an area at the female side (D2) which is greater than the area of the through holes at the male side (D3).

8. The absorbent article according to claim 1, wherein the female side faces the upper layer (A) and the male side the absorbent core (C).

9. The absorbent article according to claim 1, wherein the male side faces the upper layer (A) and the female side the absorbent core (C).

10. The absorbent article according to claim 8, wherein the sum of the areas D2 of the acquisition-distribution layer (D) is at least 50% of the total area of the acquisition-distribution layer (D)

11. The absorbent article according to claim 8, wherein ratio of the width of the segment W1 to the height H1 is below 1.

12. The absorbent article according to claim 11, wherein the width is below 500 μm.

13. The absorbent article according to claim 1, wherein the acquisition-distribution layer is a non-woven web comprising a three dimensional network of fibers.

14. The absorbent article according to claim 1, wherein the acquisition-distribution layer comprises a three-dimensional apertured, thermoplastic film.

15. The absorbent article according to claim 1 wherein the fluid absorbent core comprises at least 12% by weight of water-absorbent polymer particles.

16. The absorbent article according to claim 1, wherein the fluid-absorbent core comprises at least 50% by weight of water-absorbent polymer particles.

17. The absorbent article according to claim 1, wherein the fluid-absorbent core comprises at least 85% by weight of water-absorbent polymer particles.

18. The absorbent article according to claim 1, wherein the water-absorbent polymer particles are placed within the core in discrete regions.

19. The absorbent article according to claim 1, wherein the water-absorbent polymer particles have a centrifuge retention capacity of at least 10 g/g and an absorbency under high load of at least 7 g/g.

20. The absorbent article according to claim 1, wherein the water-absorbent polymer particles have a centrifuge retention capacity of at least 25 g/g and an absorbency under high load of at least 15 g/g.

21. The absorbent article according to claim 1, wherein the water-absorbent polymer particles have a level of extractable constituents of less than 10% by weight
